SEQUENCE LISTING (24 sequences)

SEQ ID NO: 1 - DNA, 3819 bp, Arabidopsis thaliana
  source 1..3819: mol_type = genomic DNA
agacaagaagagaagagaaaacatgttttccattagaggactgtgagattgtgaattgcatagtcgtcgtcttctggcgggaaaagaagccctagaaaaagggtgaaaggtgaaaactctacttcttcttcttcttcttcttcagagtgtgagagagatgcattggattgctaccagaaacgccgtcgtttcattcccaaaatggcggttcttcttccgctcctcatatcgcacttactcttccctcaaaccctcctccccaattctacttaatagaaggtactctgaggggatatcttgtctcagagatggaaagtctttgaaaagaatcacaacggcttctaagaaagtgaagacgtcaagtgatgttctcactgacaaagatctctctcatttggtttggtggaaggagagattgcagacatgtaagaaaccatctactcttcagcttattgaaaggcttatgtacaccaatttacttggtttggaccctagcttgaggaatggaagtttaaaagatggaaacctcaactgggagatgttgcagtttaagtcaaggtttccacgcgaagttttgctctgcagagtaggagaattttatgaggctattggaatagatgcttgtatacttgttgaatatgctggtctcaatccttttggtggtcttcgatcagatagtattccaaaggctggctgcccaattatgaatcttcgacagactttggatgacctgacacgcaatggttattcagtgtgtattgtggaggaagttcaggggccaacaccagcacgctcccgtaaaggtcgatttatttcagggcatgcacatccaggaagtccttatgtatatgggcttgtcggtgttgaccatgatcttgactttcctgatcctatgcctgttgttgggatatctcgttcagcaagggggtattgtatgatatctattttcgagactatgaaagcatattcgctagatgatggtctaacagaagaagccttagttaccaagctccgcactcgtcgctgtcatcatcttttcttacatgcatcgttgaggcacaatgcatcagggacgtgccgctggggagagtttggggaagggggtctactctggggagaatgcagtagcaggaattttgaatggtttgaaggagatactctttccgagctcttatcaagggtcaaagatgtttatggtcttgatgatgaagtttcctttagaaatgtcaatgtaccttcaaaaaatcggccacgtccgttgcatcttggaacggctacacaaattggtgccttacctactgaaggaataccttgtttgttgaaggtgttacttccatctacgtgcagtggtctgccttctttgtatgttagggatcttcttctgaaccctcctgcttacgatattgctctgaaaattcaagaaacgtgcaagctcatgagcacagtaacatgttcaattccagagtttacctgcgtctcttctgctaagcttgtgaagcttcttgagcaacgggaagccaactacattgagttctgtcgaataaaaaatgtgcttgatgatgtattacatatgcatagacatgctgagcttgtggaaatcctgaaattattgatggatcctacctgggtggctactggtttgaaaattgactttgacacttttgtcaacgaatgtcattgggcgtctgatacaattggtgaaatgatctctttagatgagaatgaaagtcatcagaatgtaagtaaatgtgacaatgtcccgaacgaattcttttatgatatggagtcttcatggcgaggtcgcgttaagggaattcatatagaggaagaaatcactcaagtagaaaaatcagctgaggctttatctttagcagtagctgaggattttcaccctattatatcaagaattaaggccaccactgcttcacttggtggcccgaaaggcgaaatcgcatatgcaagagagcatgagtctgtttggttcaaggggaaacggtttacgccatctatctgggctggtactgcaggggaagaccaaataaaacagctgaaacctgccttagactcgaaaggaaaaaaggttggagaagaatggtttacgaccccaaaggtggaaattgctttagtcagataccatgaagctagtgagaatgcaaaagctcgggtgttggaactgttgcgcgagttatccgttaaattgcaaacaaaaataaatgttcttgtctttgcatctatgcttctggtcatttcaaaagcattattttcccatgcttgtgaagggagaaggcgaaagtgggtttttccaacgcttgtcggattcagtttagatgagggcgcaaaaccattagatggtgccagtcgaatgaagctgacaggcctgtcaccttattggtttgatgtatcttctggaaccgctgttcacaataccgttgacatgcaatcactgtttcttctaactggacctaacggtggtggtaaatcgagtttgctcagatcaatatgcgcagctgctctacttggaatttccggtttaatggttccagctgaatcagcttgtattcctcactttgattccatcatgcttcacatgaaatcatatgacagccctgtagacggaaaaagttctttccaggtagaaatgtcggaaatacgatctattgtaagccaggctacttcgagaagcctagtgcttatagatgagatatgccgagggacagagacagcaaaaggcacctgtatcgctggtagtgtggtagagagtcttgacacaagtggttgtttgggtattgtatctactcatctccatggaatcttcagtttacctcttacagcgaaaaacatcacatataaagcaatgggagccgaaaatgtcgaagggcaaaccaagccaacttggaaattgacagatggagtctgcagagagagtcttgcgtttgaaacagctaagagggaaggtgttcccgagtcagttatccaaagagctgaagctctttacctctcggtctatgcaaaagacgcatcagctgaagttgtcaaacccgaccaaatcataacttcatccaacaatgaccagcagatccaaaaaccagtcagctctgagagaagtttggagaaggacttagcaaaagctatcgtcaaaatctgtgggaaaaagatgattgagcctgaagcaatagaatgtctttcaattggtgctcgtgagcttccacctccatctacagttggttcttcatgcgtgtatgtgatgcggagacccgataagagattgtacattggacagaccgatgatcttgaaggacgaatacgtgcgcatcgagcaaaggaaggactgcaagggtcaagttttctataccttatggttcaaggtaagagcatggcttgtcagttagagactctattgattaatcaactccatgaacaaggctactctctggctaacctagccgatggaaagcaccgtaatttcggaacgtcctcaagcttgagtacatcagacgtagtcagcatcttatagtttgaaacattagctgtgtttgtagttgatcatctctatgtgcaattgaacaagtcagtttgctagaactagagtagattactaagaaaccatgccgtttttcattttgagattttgcaaaacggcatgcagttcgggtaagtcggatgccgcaattaccaattttgggtcagtctgtgtaattgtcgtttcataaatccgattaacgtgtactttgaacaaaactcagcagtaaacttctttattcatcaataagtaatgctagaaattttaaccaataagaatatgaccatataactaaataa

SEQ ID NO: 2 - DNA, 3393 bp, Glycine max
  source 1..3393: mol_type = genomic DNA
atgtacagggtagccacaagaaacgtcgccgttttcttccctcgttgctgttccctcgcgcactacactccttctctatttcccattttcacttcattcgctccctctcgtttccttagaataaatggatgtgtaaagaatgtgtcgagttatacggataagaaggtttcaagggggagtagtagggccaccaagaagcccaaaataccaaataacgttttagatgataaagaccttcctcacatactgtggtggaaggagaggttgcaaatgtgcagaaagttttcaactgtccagttaattgaaaggcttgatttttctaatttgcttggcctggattccaatttgaaaaatggaagtctgaaggaaggaacactcaactacgaaatgttgcagttcaagtcaaaatttccacgtcaagtattgctctgcagagttggggatttctacgaagcttggggaatagatgcttgtgtgctcgttgaatatgctggtctaaatcccttcggtggtctgcgatcggatagcattccaagagctggttgtcctgttgtgaatcttcgacagactttagatgatttgacacaaaatggtttttcagtgtgcattgtggaggaagttcagggcccaactcaagttcgatcaagaaaacgccgctttatatctgggcacgctcatcctggaaatccttatgtatatggacttgctgcagttgatcacgatcttaactttccagaaccaatgcctgtagtaggaatatctcattctgcgaggggttattgcattaatatggtactagagaccatgaagacatattcttctgaagattgcttgacagaagaagcagttgttacgaagcttcgtacttgccaatatcattacttatttttgcatacatccttgaggcggaattcttgtggaacctgcaactggggagaatttggtgagggagggctattatggggagaatgtagttctagacattttgattggtttgatggcaaccctgtctccgatcttttggccaaggtaaaggaactttatagtattgatgatgaggttacctttcggaacacaactgtgtcttcaggacatagggctcgaccattaactcttggaacatctactcaaattggtgccattccaacagaaggaataccttctttgttgaaggttttacttccatcaaattgcaatggattaccagtattgtacataagggaacttcttttgaatcctccttcatatgagattgcatccaaaattcaagcaacatgcaaacttatgagcagtgtaacgtgttcaattccagaatttacatgtgtttcgtcagcaaagcttgtaaagctacttgaatggagggaggtcaatcatatggaattttgtagaataaagaatgtactggatgaaattttgcagatgtatagtacctctgagctcaatgaaatattgaaacatttaatcgagcccacatgggtggcaactgggttagaaattgactttgaaaccttggttgcaggatgtgagatcgcatctagtaagattggtgaaatagtatctctggatgatgagaatgatcagaaaatcaactcgttctcttttattcctcacgaattttttgaggatatggagtctaaatggaaaggtcgaataaaaagaatccacatagatgatgtattcactgcagtggaaaaagcagctgaggccttacatatagcagtcactgaagattttgttcctgttgtttctagaataaaggctattgtagcccctctcggaggtcctaagggagaaatatcttatgctcgggagcaagaagcagtttggttcaaaggcaaacgctttacaccgaatttgtgggctggtagccctggagaggaacaaattaaacagcttaggcatgctttagattctaaaggtagaaaggtaggggaggaatggtttaccacaccaaaggtcgaggctgcattaacaaggtaccatgaagcaaatgccaaggcaaaagaaagagttttggaaattttaaggggactcgctgctgagttgcaatacagtataaacattcttgtcttttcttccatgttgcttgttattgccaaagctttatttgctcatgcaagtgaagggagaagaaggagatgggtctttcccacgcttgtagaatcccatgggtttgaggatgtgaagtcattggacaaaacccatgggatgaagataagtggtttattgccatattggttccacatagcagaaggtgttgtgcgtaatgatgttgatatgcaatcattatttctgttgacaggaccgaatggtggtgggaaatcaagttttcttaggtcaatttgtgctgctgcactacttgggatatgtggactcatggttcctgcagaatcagccctaattccttattttgactccatcacgcttcatatgaagtcatatgatagtccagctgataaaaagagttcctttcaggttgaaatgtcagaacttcgatccatcattggcggaacaaccaacaggagccttgtacttgttgatgaaatatgccgaggaacagaaactgcaaaagggacttgcattgctggtagcatcattgaaacccttgatggaattgggtgtctgggtattgtatccactcacttgcatggaatatttactttgcccctaaacaaaaaaaacactgtgcacaaagcaatgggcacaacatccattgatggacaaataatgcctacatggaagttgacagatggagtttgtaaagaaagtcttgcttttgaaacggctaagagggaaggaattcctgagcatattgttagaagagctgaatatctttatcagttggtttatgctaaggaaatgctttttgcagaaaatttcccaaatgaagaaaagttttctacctgcatcaatgttaataatttgaatggaacacatcttcattcaaaaaggttcctatcaggagctaatcaaatggaagttttacgcgaggaagttgagagagctgtcactgtgatttgccaggatcatataaaggacctaaaatgcaaaaagattgcattggagcttactgagataaaatgtctcataattggtacaagggagctaccacctccatcggttgtaggttcttcaagcgtctatgtgatgttcagaccagataagaaactctatgtaggagagactgatgatctcgagggacgggtccgaagacatcgattaaaggaaggaatgcatgatgcatcattcctttattttcttgtcccaggtaaaagcttggcatgccaatttgaatctctgctcatcaaccaactttctggtcaaggcttccaactgagcaatatagctgatggtaaacataggaattttggcacttccaacctgtatacataa

SEQ ID NO: 3 - DNA, 2025 bp, Glycine max
  source 1..2025: mol_type = genomic DNA
atggaattttgtagaataaagaatgtacttgatgaaattttgctgatgaacaaaacctctgagctcaatgacatattaaaacatttaattgaccccacatgggtggcaacggggctagaaattgactttgaaaccttggttgctggatgtgaagttgcatctactaagattggtgatataatctctctggatggtgggaatgatcagaaaatcaactctttctctcttattcctcatgaattttttgaggatatagagtctaaatggaaaggccgaataaaaagaatccacatagatgatgtatttactgctgtggaaaaagcagctgaagccttgcatatagcagtcactgaagattttgttcctattctttctagaataaaggctactgtatccccacttggaggtcctaagggagagatatcttatgctcgggagcatgaggcagtttggttcaaaggcaaacgctttactccgaatttgtgggctggtagccctggagaggaacaaattaaacagcttagccatgctttagattctaaaggaaaaaaggcaggggaggaatggtttaccacactaaaggtggaggctgcattaacaaggtaccatgaagcaaatggcaaggcaaaagaaagagttttggaaattttaaggggacttgctgctgaattgcaatacaatataaacatccttgtcttttcttccacgttgcttgttattgccaaagctttatttgctcatgcaagtgaagggagaagaaggagatgggtctttcccacacttgtagaatcccatgggtttgaggatgtgaagtcattgaacaaaatccatgggatgaagatagttggtttattaccttattggctccacgtagcagaaggtgttgtgcgtaacgatgttgatatgcaatcattatttcttttgacaggaccaaatggtggtgggaaatcaagtttactgcggtcaatttgtgctgctgcactacttggaatttgtggactcatggttcctgccgaatcagcccatattccttattttgactccatcatgctacatatgaattcatatgatagtccagccgataaaaagagttcctttcaggtggaaatgtcagaacttcgatccatcattggtggaaccaccaaaaagagccttgtacttattgatgaaatttgtcgaggaacagaaacagccaagggcacttgtattgctggtagtatcattgaaactcttgatagaattgggtgtttgggtatcgtatccactcacttacatggaatatttactttacccctaaacataaacaacactgtgcacaaagcaatgggcacaacatccattgatggacaaacaattcctacctggaagctaacggatggagtttgtagagaaagtcttgcttttgaaactgccaggagagaaggagttcctgagcttattatcagaagagctgaatatatttatcagtcagtttatgctaaggaaaaggaactgctttctgcagaaaaatcctcaaatgaaaaaaagtattctacttatatcaatgtcagtaatttgaatggaacacatcttccttcaaagaggttcttatcaggagctaatcaaacggaagttttacgcgaggaagttgagagtgctgtcactgtgatatgccaggaccacataatggaacaaaaaagcaaaaacattgcattagagcttactgggataaaatgtctccaaatcaggacaagggagcagccacctccgtcagttgtaggttcttcaagtgtctatgtgatgttcagaccagataaaaaactctatgttggagagactgatgatcttgagggtcgtgtccgagcacatcgattgaaggaaggaatgcatgatgcatcgtttctttattttcttgtccctggaaaaagcttggcatgccaattagaatctttgctcatcaaccagctttctagtcgaggcttccaactgaccaatacagctgatggtaaacataggaattttggcacttccaacctgtatgcataa

SEQ ID NO: 4 - DNA, 3636 bp, Brassica napus
  source 1..3636: mol_type = genomic DNA
atgcactggattgccaccagaaacgccgtcgtttcgctccctagatggcgttccttcgccttcctcttccgctcgccatttcgcactcactcttccctcaaaccctcccctcttctacttaatacaaggtactctgagaggagatactgtttaggagatggaaaggctgtgaaaggaatcactacggcttcttctaagaaagttaagaccaagtctactgatgttctcactgacaaagatctctctcatttgctctggtggaaggagagattgcagacatgtaagaaaccatctactcttcaacttatcgaaagacttatgtacaccaatctacttggtctggaccccagcctgaggaatggaagtctaaaagacggaaacctcaactgggagatgttgcagtttaagtcaaggtttccacgagaagttttgctctgcagagtgggagacttctatgaggctattgggatagatgcatgtatactggttgagtatgctggtctaaatccttttggtggtcttcgctcagatagtgctccaaaggctggctgcccagttgtgaatcttcgacagactttggatgatctaacacgcaacggttattcagtgtgtattgtggaggaagttcaggggccaacaccagctcgttctcgaaaaggtcgattcatttcagggcatgcacatccaggaagtccttatgtatatgggctcgttggtgtggaccacgatcttgactttccggagcctatgccggtcgttgggatatctcgttcagcaagggggtattgcatgatatctatcttcgaaactatgaaagcatattcactagatgatggtctaacagaagaagctctggtcaccaagctccgcacccgtcgctgtcatcatcttttcttacatgcatcattgaggcacaatgcatcagggacatgccggtggggagagtttggggaaggaggtttactctggggagagtgtagtgggaggaattttgaatggtttgaaggagatactctttccgagctcttattaaaggtcaaagatgtttatggtcttgatgatgaagtttcctttagaaatgtcaatgtaccttcagaaaaccgtccacgtccattgcatctcggaacggctacacaaattggtgccttacctactgaaggaataccttgtttgttgaaggtgctgcttccatctacgtgcagtggcctgccttctttgtatgtccgggatctacttctaaaccctcctgcttatgatattgctctgaagatccaagaaacgtgcaagctcatgagcacaataacatgctcagttccggagtttacctgtgtttcatctgctaagcttgtgaagcttcttgaacaacgggaagccaactacattgagttctgccggataaaacatgtgcttgatgaagtattacatatgcatagacatcctgaacttgtggaaatactgaagttactgatggagcctacttgggtggctactggtttgaagatcgactttgaaacttttgtcaatgaatgccattgggcttctgactcgattggtgaaatgatctctctggatgacgatgaaagtcatcagaacgttagtaaatgtgctaatatcccgaacgagtttttttatgatatggagtcttcatggcgtggtcgcgttaagggaatccatatagaggaagaaatcacacaagtggccaaatcggcagaggctttatctttagcggtaactgaagattttcaccctattatatcaagaatcaaggccatggctgcatcacttggtggctcaaagggagaaattgtgtatgcaaaagaacatgagtctgtctggttcaaagggaaacggtttaccccatctgtgtggggtggtactgcaggagaagaacaaatcaaacagctgaagcctgcttttgactcgaaagggaaaaaggttggagaagaatggtttacgagtcaaaaggtggaagctgctttagtcaggttatatcatgaagctactgagaatgcaaatgcccgggtgttggagctgttgagggaattatctactaaactgcaaacaaaaataaacgttcttgtctttgcatctatgcttctcgtcattgcaaaagctttattttctcatgcttgtgaagggagaagacgaaaatgggttttcccaactcttgttggattcagtacagatgaggccgcaaatccattagatggtggtgccactcgaatgaagctgactgggctatcaccttattggtttgatgtagcttctggaactgctgttcacaatacggtcgacatgcaatcactgtttcttctaaccggacctaatggtggtggtaaatcgagtttgctcagatcgatatgcgcagctgctttgcttggaatctgtggttttatggttccagctgaatcagcttatatccctcacttcgattccatcatgcttcatatgaaatcttatgacagtcctgtagatgggaagagttcttttcaggtggaaatgtcggagatacggtctattgtaagccaggctacttcaagaagcctagtgcttatagatgagatctgcagagggacagagacagctaaaggcacctgtattgctggtagtgtgatcgagaatcttgacgcaagtggttgcttgggtattgtttctacacatctccatggaatcttcgatttgcctcttacggccaaaaacgtcacgtataaagcaatgggagcacagaatgtggaagggcaaacaaaaccaacatggaaactgacagatggagtttgcagagagagtcttgcttttgaaacagctaagagagaaggtgttccggagacaattatccaaagagccgaagctctgtacatctccgtttatgccaaagacgcatcgtttggggttgtcaggccaaacaaaacggagacttcatcgaacagtggccgcgaaatcagcaaaccagtcagctctgagagaagcttagagaaggacttggcaaaagctatccttaagatttgtgggataaagatgaatgagcctgtaggtttagaatgtctttcaataggtgctcgagagcttccacctccatctacagttggttcatcatgcgtgtatgtgatgaagagaccagataagagattgtacattggacagacggatgatcttgaaggaagaatacgtgcgcatagggcaaaggaaggactgcaagggtcaagtttcctataccttgtggtacaaggtaagagtatggcttgtcagctagagacccttttgattaaccagctccatgagcaaggctattctctagctaacttagcagatggaaagcaccgcaattttgggacgtcatcaagcttgactgcgtcagatgtagtcagtatctcctagtttcaacattagcattgagtgtagaaactgataatctcgctctctgtttgggtttgtgctgcagttgcacattttagtttggcttaggaagttagaattagagtagattaaaacctaggcatatgccataaggaaagaaagaaatgtacgccgttttgcttgttgaggtttttggagatattacaaacggcaggcagttggcaattacccttattttggtcttcgggtgtaattgtcacattttcattcaaccgagatttg

SEQ ID NO: 5 - DNA, 3441 bp, Brassica napus
  source 1..3441: mol_type = genomic DNA
atgcactggattgccaccagaaacgccgtcgtttcgctccctagatggcgttccttcgccttcctcttccgctcgccatttcgcacccactcttccctcaaaccctccccacttcttctacttaatacaaggtactctgagaggagatactgtttaggagatggaaagtctgtgaaaggaatcactacggcttcttctaagaaagttaagaccaagtctactgatgttctcactgacaaagatctctctcatttgctatggtggaaggagagattgcagacatgtaagaaaccatctactcttcaacttatcgaaaggcttatgtacaccaatctacttggtttggaccccagcttgaggaatggaagtcttaaagacggaaacctcaactgggagatgttgcagtttaagtcaaggtttccacgtgaagttttgctctgcagagttggagacttctatgaggctattggaatagatgcttgtatactcgttgaatatgctggtttaaatccttttggtggtcttcgttcagatagtgttccaaaggctggctgcccagttgtgaatcttagacaaactttggatgacctaacacgcaatggtttttcagtgtgtattgtggaagaaattcaggggccaacaccagcacgttctcgtaaaggtcgattcatttcagggcatgcacatccaggaagtccttatgtctatgggctcgttggtgttgaccatgatcttgactttccggagcctatgcctgtggttgggatatctcgttcagcaaggggctactgtatgatatctatcttcgagactatgaaagcatattcactagatgatggtctaacagaagaagctctggtcaccaagctccgcacccgtcgctgtcatcatcttttcttacatgcatcattgagacacaatgcatcaggaacatgccggtggggagagtttggagaagggggtctcctctggggagaatgtagtggcagaaattttgaatggtttgaaggagatactctttccgagctcttaacaaaggtcaaagatgtttatggtcttgatgatgaagtttcctttagaaatgtcaatgtacctttagaaaaccggccacgtcctttgcatcttggaacggctacacaaattggtgccttacctactgaaggaataccttgtttgttgaaggtgctacttccatctacgtgcagtggcctgccttctttgtatgtccgggatcttcttctaaaccctcctgcttatgatattgctctgaaaatccaagaaacgtgcaagctcatgagcacaataacatgctcagttccggagtttacctgtgtttcctctgctaagcttgtgaagcttcttgaacagcgggaagccaactacattgagttctgccggataaaaaatgtgcttgatgaagtattacacatgcacagacatcctgagcttgtggaaatactgaagttattgatggaacctacttgggtggctactggtttgaagattgactttgaaacttttgtcaatgaatgtcattgggcttctgattcaattggtgaaatgatctcattagatgacgatgaaagtcatcagaacgttagtaaatgtgctaatgtcccgaacgagttcttttacgatatggagtcttcatggcgtggtcgcgttaagggaatccatatagaggaagaaatcacacaagtggccaaatcggcagaggctttatctttagcggtaactgaagatttccaccctattatatcaagaatcaaggctatggctgcatcacttggtggctcaaagggagaaattgtgtatgcaaaagaacatgagtctgtttggttcaaagggaaacggtttaccccatctgtatggggtggtactgcaggggaagaacaaattaaacagctgaaacctgcttttgactccaaagggaaaaaggttggagaagaatggtttacaactcaaaaggtggaaactgctttagtcaggttatatcatgaagctagtgagaacgcaaatgcccgggtgttggagctgttgagggagttatctgctaaattgcaaacaaaaataaacgttcttgtatttgcatctatgcttctcgtcattgcaaaagcgttattttctcatgcttgtgaagggagaagacgaaagtgggtttttccaactcttgttggtttcagtacagatgaggccgcaaatccattagatggtggtgccactcgaatgaagctgactgggctatcaccttattggtttgatgtagcttctggaactgctgttcacaatacggtcgacatgcaatcactgtttcttctaactggacctaatggtggtggtaaatcgagtttgctcagatcgatatgcgcagctgctttgcttggaatctgtggttttatggttccagctgaatcagcttatatccctcacttcgattccatcatgcttcatatgaaatcttatgacagtcctgtagatgggaagagttcttttcaggtggaaatgtcggagatacggtctattgtaagccaggctacttcaagaagcctagtgcttatagatgagatctgcagagggacagagacagctaaaggcacatgtattgctggtagtgtgatcgagagtcttgacgcaagtggttgcttgggtattgtgtctacacatctccatggaatcttcgatttgcctcttacggccaaaaacgtcacgtataaagcaatgggagcagagaatgtggaagggcaaacaaaaccaacatggaaactgacagatggagtttgcagagagagtcttgcgtttgaaacagctaagagagaaggtgttccggagacaattatccaaagagccgaagctctttacatctccgtttatgccaaagacgcatcgtttggggttgtcaggccaaacaaaacggagacttcatcggacaatgagatcagcaaaccagtcaggtctgagagaagcttggagaaggacttggcaaaagctatccttaagatttgtgggataaagatgaatgagcctgtaggtttagaatgtctttcaataggtgctcgagagcttccacctccatctacagttggttcatcatgcgtgtatgtgatgaagagaccagataagagattgtacattggacaggtatacttccatatcacactattaagtaatcttctgaggatttgttgtgttaatgtaatgaaagtgcagacggatgatcttgaaggaagaatacgtgcgcatagggcaaaggaaggactgcaagggtcaagtttcctataccttgtggtacaaggtaagagtatggcttgtcagctagagacccttttgattaaccagctccatgagcaaggctactctctagctaacttagccgatggaaagcaccgcaattttgggacgtcatcaagcttgactgcgtcagatgtagtcagtatctcctag

SEQ ID NO: 6 - DNA, 3676 bp, Solanum lycopersicum
  source 1..3676: mol_type = genomic DNA
atgtattgggttacggcaaaaaacgtcgtcgtttcagttccccgttggcgttcactgtcccttttcctccgtccaccacttcgccggcgtttcttatctttctctccacatactctgtgccgagagcagatacgttgcgtgaaggagcggaagttttttgccacaacggcaaaaaaactcaaacaaccaaaaagtattccagaggaaaaagactatgttaatattatgtggtggaaagagagaatggaattcttgagaaagccttcttccgctcttctggctaagaggcttacatattgtaacttgctgggtgtggatccgagtttgagaaatggaagtcttaaagagggaacacttaactcggagatgttgcagttcaagtcaaaatttccacgtgaagttttgctctgtagagtaggtgatttttatgaagctattggattcgatgcttgtattcttgtggaatatgctggtttaaatccatttggtggcctgcactcagatagtataccaaaagctggttgtccagttgtgaatctaagacagacgcttgatgatctcacacgtaatggtttctctgtgtgcgtcgtggaggaagttcagggtccaactcaagctcgtgctcgtaagagtcgatttatatcagggcatgcacatccaggcagtccctatgtttttggccttgttggagatgatcaagatcttgattttccagaaccaatgcctgttgttggaatatcccgttcagcgaaggggtattgcattatctctgtttacgagactatgaagacttactctgtggaagatggcctaactgaagaagccgtagtcaccaaacttcgtacttgtcgatgccatcatttttttttgcataattcattgaagaacaattcctcaggaacatcgcgttggggagagtttggtgaaggtggacttttgtggggagaatgtaatgctagacagcaggaatggttggatggcaatcctatcgatgagcttttgttcaaggtaaaagagctttatggtctcaatgatgacattccattcagaaatgtcactgttgtttcagaaaataggccccgtcctttacaccttggaactgccacacaaattggtgctattccaaccgaagggattccatgtttgttaaaggtgttgcttcctcctcattgcagtggtctaccagtcctgtatattagggatcttcttttaaatccaccagcctatgagatttcttcagacattcaagaggcatgcagacttatgatgagtgtcacatgttcaattcctgattttacctgtatttcatctgcaaagctggtcaagctgcttgagttgagggaggcaaatcacgttgagttctgcaaaataaagagcatggtcgaagagatactgcagttgtatagaaattcagagcttcgtgctattgtagagttactgatggatcctacttgggtggcaactgggttgaaagttgattttgatacactagtaaatgaatgtggaaagatttcttgtagaatcagtgaaataatatccgtacatggtgaaaatgatcaaaagattagttcctatcctatcatcccaaatgatttctttgaagatatggagttgttgtggaaaggccgtgtcaagaggatccatttggaggaagcatatgcagaagtagaaaaggctgcggatgctttatctttagccataacagaagatttcctacctattatttcaagaataagggccacgatggccccacttggaggaactaaaggggagattttgtatgcccgtgagcatggagctgtatggtttaagggaaagagatttgtaccaactgtttgggctggaaccgctggagaagaacaaattaagcaactcagacctgctctagattcaaaggggaagaaggttggagaagaatggttcactacaatgagggtggaagatgcaatagctaggtatcacgaggcaagtgctaaggcaaagtcaagggtcttggaattgctaaggggactttcttctgaattactatctaagatcaatatccttatctttgcatctgtcttgaatgtgatagcaaaatcattattttctcatgtgagtgaaggaagaagaagaaattggattttcccaacaatcacacaatttaacaaatgtcaggacacagaggcacttaatggaactgatggaatgaagataattggtctatctccttattggtttgatgcagcacgagggactggtgtacagaatacagtagatatgcagtccatgtttcttttaacaggtccaaatggtgggggcaaatcaagcttgctgcgttcgttgtgtgcagctgcattgctaggaatgtgtgggttcatggttccagctgaatcagctgtcattcctcattttgactcaattatgctgcatatgaaatcatatgatagtcctgttgatggaaaaagttcatttcagattgaaatgtctgaaattcggtctctgattactggtgccacttcaagaagtcttgtacttatagatgaaatatgtcgaggaacagaaacagcaaaagggacatgtattgctggaagtgtcatagaaaccctggacgaaattggctgtttgggaattgtatcaacccacttgcatggaatatttgatttacccctgaaaatcaagaagaccgtgtataaagcaatgggagctgaatatgttgacggtcaaccaataccaacttggaaactcattgatgggatctgtaaagagagtctagcatttgaaacagctcagagagaaggaattccagaaatattaatccaaagagcagaagaattgtataattcagcttacgggaatcagataccaaggaagatagaccaaataagacctctttgttcagatattgacctcaatagcacagataacagttctgaccaattaaatggtacaagacaaatagctttggattctagcacaaagttaatgcatcgaatgggaatttcaagcaagaaacttgaagatgctatctgtcttatctgtgagaagaagttaattgagctgtataaaatgaaaaatccgtcagaaatgccaatggtgaattgcgttcttattgctgccagggaacagccggctccatcaacaattggtgcttcaagtgtctatataatgctaagacctgacaaaaagttgtatgttggacagactgatgatcttgagggcagagtacgtgctcatcgcttgaaggagggaatggaaaacgcgtcattcctatatttcttagtctctggcaagagcatcgcctgccaattggaaactcttctaataaatcaacttcctaatcatggttttcagctaacaaacgttgctgatggtaagcatcgaaattttggcacaaccaatctctccccagaaccttcatccgcgcttagatgaactcatcattctgctatggaagagctcaagttggtgtataaaatttgtattagtttttgaaaattttcaagggatgagtaattcaactttcagtggctgcagcaattttgggttagttttagcttctccacatgtatatcacatcatcacctgtgaaactaagggttaattaagccttgtaacacttttgatatctaagttttagtgtcctgacaaagatgtggtctactagctaatgaagtgcgtaaaaattatgagagac

SEQ ID NO: 7 - DNA, 3399 bp, Oryza sativa
  source 1..3399: mol_type = genomic DNA
atggccattcagcggctgctcgcgagctcgctcgtggccgccacgccgcggtggcttcccgtcgccgccgactcgtttctccggcgccgccaccgccctcgctgctccccgctccccgcgctgctatttaacaggaggtcctggtctaaaccaaggaaagtctcacgaagcatttccattgtgtctaggaagatgaacaaacaaggagatctctgtaatgaaggcatgctgccacatattctgtggtggaaagagaaaatggagaggtgcaggaaaccatcatcaatgcaattgactcagagacttgtgtattcaaatattttaggattggatccaactttaagaaatggaagcttgaaggatggaagcctgaacacggaaatgttgcaattcaaatcgaagtttcctcgtgaagttctactttgcagagtgggagatttctacgaggctgttgggtttgatgcatgtatccttgtggagcatgcaggcttaaatccttttggaggcttgcgttctgatagtattccaaaagctggatgtccagtcatgaatttgcggcagacattggatgatttgactcgatgtggttactctgtgtgcatagttgaagaaattcaaggcccaacccaagctcgtgctaggaaaggccgatttatttctggccatgcacatcctggtagtccttatgtatttggtcttgctgaagtagaccatgatgttgagttccctgatccaatgcctgtagttgggatttcacgatctgcaaaaggctattgcctgatttctgtgctagagacaatgaaaacatattcagctgaggagggcttaacagaggaagcagttgttactaagcttcgcatatgccgttatcatcatctataccttcatagttctttgaggaacaattcttcaggcacatcacgctggggagaatttggcgaaggtgggctattgtggggagagtgcagtggaaaatcttttgagtggtttgatggtaatcctattgaagaactgttatgcaaggtaagggaaatatatgggcttgaagagaagactgttttccgtaatgtcagtgtctcattggaagggaggcctcaacccttgtatcttggaacagctactcaaattggggtgataccaactgagggaatacccagtttgctaaaaattgttctccctccaaactttggtggccttccatcattgtatattagagatcttcttcttaaccctccatcttttgatgttgcatcatcagttcaagaggcttgcaggcttatgggtagcataacttgctcgattcctgaatttacatgcataccggcagcaaagcttgtgaaattactcgagtcaaaagaggttaatcacatcgaattttgtagaataaagaatgtcctcgatgaggtgttgttcatgggtagcaatgctgagctttctgctatcctgaataaattgcttgatcctgccgccatagttactgggttcaaagttgaagccgatatactagtgaatgaatgtagctttatttcacaacgtatagctgaagtaatctctttaggtggtgaaagtgaccaggcaataacttcatctgaatatattccgaaagagttcttcaatgatatggagtcatcttggaagggacgtgtaaaaagggtgcatgctgaagaggagttctcaaatgttgatatagctgctgaggcactgtcaacagcggtcattgaagattttctgccaattatttcaagagtaaaatctgtgatgtcctcaaatggaagttcgaagggagaaatcagttatgcaaaagagcatgaatctgtttggtttaaagggaggcgattcacaccaaatgtgtgggccaacactcctggtgaactacagataaagcaattgaagcctgcaattgactcaaaaggtagaaaggtcggagaagaatggttcaccactatcaaagttgagaatgctttaaccaggtaccatgaagcttgtgataatgcaaaacgtaaagttcttgagttgttgagaggactttcaagtgaattgcaggacaagattaatgtccttgtcttttgctcaacgatgctcatcataacaaaagcactttttggtcatgttagtgaaggacgaagaaggggttgggtgcttcctactatatctcccttgtgtaaggataatgttacagaggaaatctcaagtgaaatggaattgtcaggaacttttccttactggcttgatactaaccaagggaatgcaatactgaatgatgtccatatgcactctttgtttattcttactggtccaaacggtggtggtaaatccagtatgctgagatcagtctgtgctgctgcattacttggaatatgtggcctgatggtgccagctgcttcagctgtcatcccacatttcgattccatcatgctgcatatgaaagcatatgatagcccagctgatggtaaaagttcgtttcagattgaaatgtcagagatacgatctttagtctgccgagctacagctaggagtcttgttctaattgatgaaatatgtaggggcacagaaacagcaaaaggaacatgtatagctggtagcatcattgaaagactcgataatgttggctgcataggcatcatatcaactcatttgcatggcatttttgaccttccactgtcactccacaatactgatttcaaagctatgggaaccgaaatcatcgataggtgcattcagccaacatggaaattaatggatggcatctgtagagagagtcttgcttttcaaacagccaggaaagaaggtatgcctgacttgataattagaagagctgaggaactatatttggctatgagcacaaacagcaagcagacatcatcagctgtccaccatgaaatatccatagccaactctactgtaaatagcttggttgagaagcctaattacctgagaaatggactagagcttcaatctggttccttcggattactaagaaaagaaattgagagtgttgttaccacaatatgcaagaagaaactgttggatctctacaacaaaaggagcatctcagaactgattgaggtggtctgtgttgctgtgggtgctagggagcaacccccaccttcaactgttggcaggtccagcatttatgtaattatcagacgtgacagcaagctctatattggacagacggatgatcttgtgggtcgacttagtgctcacagatcgaaggaaggtatgcaggatgccacgatattatatattttggtacctgggaagagcattgcatgccaactggaaactcttctcataaatcagctacctttgaaaggtttcaagctcatcaacaaggcagatggcaagcatcgaaatttcggtatatctcttgtcccaggagaggcaattgccgcatag

SEQ ID NO: 8 - DNA, 4422 bp, Sorghum bicolor
  source 1..4422: mol_type = genomic DNA
ccccttcccggcctgggagcggcctggcgagtggcgactggcgtcttccgcggttccccccagttcgcctccttcactgccgcccgcgcgccccgacacgcctgaaaaaccccacctctcccctccgctccgcctctctcgccctccacttcccacgccccacgccgcctgccattccagttccagcgtggactcgacgccagcgcggagacgcgcgtctcgaagcactagccccccgttgttctgccgcgccggcgcgccggcgccatgcaccgggtgctcgtgagctcgctcgtggccgccacgccgcggtggctccccctcgccgactccatcctccggcgccgccgcccgcgctgctctcctcttcccatgctgctattcgaccggagggcttggtccaagccaaggaaggtctcacgaggcatctcagtggcgtctaggaaagctaacaaacagggagaatattgtgatgaaagcatgctatcgcatatcatgtggtggaaagagaaaatggagaagtgcagaaaaccatcatccgtacaattgactcagaggcttgtgtattcaaatatattagggttggatccaaatctaagaaatggaagcttgaaagatggaaccctgaacatggagattttgctatttaaatcaaaatttcctcgtgaggttctactttgcagagtaggagacttctatgaagctattggttttgatgcctgtattctcgtagagcatgcaggcttaaatccttttggaggtttgcgttctgacagtatccctaaagctgggtgtccagtcgtgaatttacggcagacattggatgatttgactcgatgtggttattctgtgtgcatagttgaggaaattcaaggcccaacacaagcccgttcccggaaaagtcgatttatttctgggcatgcccatcctggtagtccttatgtatttggtcttgctgaagtagaccatgatgtagagttccctgatccgatgcctgttgttgggatttcacattctgcaaaaggttattgcttgatatctgtgctagagacaatgaaaacttattcagctgaggagggcttaacagaagaggctattgttactaagctccgcatatgtcgttatcatcatctataccttcacaattctttgaagaataattcttcagggacatcacgctggggtgaattcggtgaaggagggctcttgtggggagagtgcagtgggaagtcctttgagtggtttgatggtttacctattgaagaacttttatgcaaggtacgggaaatatatggccttgatgagaaaactgtttttcgcaatgtcaccgtctcattggaaggcaggccccaacctctttatcttggaactgctactcaaattggagtcataccaactgagggaataccgagtttgctaaaaatggcactcccttcaagttgtggcgggcttccatcaatgtatattagagatcttcttcttaatcctccatcatttgatgttgcggcagcggtccaagaggcttgcaggcttatggggagcataacttgttctgttcctgaatttacttgcatatcagcagcaaagcttgtgaagctacttgagtctaaagaggtcaatcacattgaattttgtagaataaaaaatgtccttgatgagattatgctcatgaacaggaatgctgagctttctgcaatcctgaacaaattgctggtacctggttctgtggctactggtttgaaagttgaagctgatatgctaatgaacgaatgtagcattatttcacaacgaataggtgaagtgatttctttaggggttgaaagtgatcaggccataacctcattggaatatattccaaaggagttcttcaatgatatggagtcatcttggaaggggcgtgtgaaaagaatccatgctgaagaagagtttgcaaatgttgatagagctgctgaggcattatcaattgcggtcattgaagattttatgccaattatttcaagggtgaaatctgtagtgtcctcaaatggaggttcgaaaggagaaatctgttatgcaaaagaacatgaagctgtttggtttaaaggaaagcgattcacaccaactgtatgggctaacacacctggtgagcagcaaataaaacaactgaagcctgcaattgattcgaaaggcagaaaggttggggaggaatggtttacaacaagcaaagttgagaatgctttagccaggtaccatgaagcttgtgataatgcaagaaataaagttgttgagctgttgagagggctttcaagtgaattgcaggacaaaattaacatacttgtcttttgctcaacactgctcatcattgcaaaagcactttttggtcatgttagtgaggctcggagaagaggctggatgcttcctactatatttcccttgtcaaaggactgtgttgcagaggaaagttcaaatgcaatggatttagtaggactctttccttactggcttgatgttaatcaaggaaatgcaatattgaatgatgtccacatgcactctttatttgttcttactggtccaaatggtggtggtaaatctagtatgttgcgatcagtctgtgcagctgcgctgcttggaatatgtggcctgatggtaccttcaacttcagctgtaatcccgcattttgattccattatgctgcatatgaaagcctacgatagcccagccgatgggaaaagttcatttcagattgaaatgtcggagatacgtgctttagtcagccgagctactgctaggagtcttgtcctgattgatgaaatatgtaggggcacagaaactgcaaaaggaacctgtattgctggtagcatcatcgaaaggctggataatgttggctgcctaggcatcatatcaactcacctgcatgggatttttgacttgcctctctcactcagcactactgatttcaaagctatgggaactgaagtggtcgacgggtgcattcatccaacatggaaactgatggatggcatctgtagagaaagccttgcttttcaaacagccaggagggaaggcatgcctgagttcataatcagaagggctgaggagctatatttgactatgagtacaaataacaagcagaccgcatcaatggtccacaatgagcctcgtaatgacagccccagtgtaaatggcttggttgagaagcctgaatatctgaaatacagactagaaattctgcctggtacctttgagccgttgcggagggaagttgagagtgctgttactatgatatgcaagaaaaaactgttggatctttacaataaaagtagcatcccagaactggttgaggtggtctgtgttgctgtaggtgctagagagcaaccaccaccttccactgttggcaggtctagcatctatgtgattatcagaagcgacaacaagctttatgttggacagacggatgatcttctggggcgccttcacgcccacagatcgaaggaaggcatgcaggatgctacgatattatacatcttggttcctggcaagagcgttgcctgccagctggaaacccttctcataaatcagcttccttcgaggggcttcaagctcatcaacaaggcagacggaaagcataggaacttcggtatatctcgaatctctggagaggcaatcgccacccagctaaactaatcagctaaagatctaatttagttagtcttgacgctagtgagtctcattttgcatccttcatctcttttgcttttggctactcaataggaggcaggaactaactgacaccatatgccgccccaattttgtgagatgaattatcagtggtgctagccttgtgcatagtaggggcctagggggcgatcttcccttgtctaagcatgtagtacggtgcaaatgattagcaatgcaatgacactatttctgcaacagaccttgtgtgctagttgattcgtttgttcaatcagtgcttttgctcccactgacgtgatagcctcctgatcacttcacgtattggtagctgggcgctcctttctaggtcaaaacaaaacattggcgtggattagattccagggcacacttgctttgggagctctatgaaaatggggacgggtgctccctacatgttcagtgtcactccgaatattttacgagaaaagctgcttcttttagcttgaattgccccaattttgtgagatggattgatggtggtgctagccttgtgcatagtaggggcatctttctattgtctaaggatgtagtgcaatgcaaatgattagcaatgcaatgacactatttgagcaacagaccttgtgtagttgattcgtttgttcaatcagtgcttttgctcccagtgacgtgatagcgtgatcacttcacgagttcacgtagtacgggcgctcctttctaggtcaaaaacaaaacattggcatggattag

SEQ ID NO: 9 - DNA, 4225 bp, Zea mays
  source 1..4225: mol_type = genomic DNA
gccagcgcccggggaccgtgagcccgtgaccccagcgggcccaaccagtcaaaaccgcaagagagagtcgtgaggcgtgcgcactaccccgagaaacgtgcgacgggaacctccgcggttccccaagttcgcctccttcactactctcgcgccccggcacgcctgaaaaaccccacccctcctgccgctccgcctctcccatcacttcccacgcccctcgccgcctcccattccagcgtggacacgacgccactcgccagcacggagacgcgcgcctcgaagcactactgcactagccagccgtcgttcttccgcgccggcgccatgcaccgggtgctcgtgagctcgcttgtggccgccacgccgcgatggctgcccctcgccgactccatcctccggcgccgccggccgcgctgctcccctcttcccgtgctgatgttcgatcggagggcttggtccaagccaaggaaggtctcacgaggcatttcagtggcgtccaggaaagctaacaaacagggagaatactgtgatgaaagtatgctgtcgcatatcatgtggtggaaagagaaaatggagaggtgcagaaaaccatcatccatacaattgactcagaggcttgtgtattcaaatatattagggttggatccgaatttaagaaacggaagcttgaaagatggaaccctgaacatggagattttggtatttaaatcaaaatttcctcgtgaggttctactttgcagagtaggagatttctatgaagctatcggttttgatgcctgtattctcgtagagcatgcaggcttaaatccttttggaggtttgcgttccgacagtattcctaaagctgggtgtccagtcgtgaatttacggcagacattggatgatttgactcgatgtggttattccgtgtgcatagtcgaggaaattcaaggcccaactcaagcccgtgctcggaaaagtcgatttatttctgggcatgcccatcctggtagtccttatgtatttggtcttgctgaagtagaccatgatgtagagttccctgatccgatgcctgttgttgtcaaatactgaatttcatgaggccactggatgctgatttgagatagcacatcatggtgttctcattcatcagggatttcacattctgcaaaaggttattgcttgatatctgtgctagagacaatgaaaacttattcagctgaggagggcttaacagaggaggctattgttactaagctccgcatatgtcgttatcaccatctataccttcacaattctttgaagaataattcttcagggacatcacgctggggtgaattcggtgaaggtgggctcttgtggggagagtgcagtgggaagtcctttgagtggtttgacggttcacctattcaagaacttttatgcaaggtacgggaaatatatggccttgatgagaaaacggtttttcgcgatgtcaccgtctcattggaaggcaggccccaacctcttcatcttgggactgctactcaaattggagtcataccaactgagggaataccgagtttgttaagaatggtgcttccttcaaattgtggcgggcttccatcaatgtatattagagatcttcttcttaatcctccatcatttgaggttgcagcagcgatccaagaggcttgcaggcttatgggcaacataacctgctccattcctgaatttacatgcatatcagcagcaaagcttgtgaaactacttgagtcgaaagaggtcaatcacattgaattttgtagaataaaaaatgtccttgatgagattatgctcatgaacagggatgctgagctttctgcaatcctgcatgaattactggtacctgcttctgtggctactggtttcaaagttgaagctgatatgctaatgaacggatgtagcattatttcacaacgaatagctgaagtgatttctttaggtgttgaaagtgatcaggcaataacttcattggaatatattccaaaggagttcttcaatgatatggagtcatcttggaaggggcgcgtgaaaaggatccatgctgaagaagagtttgcaaatgttgatagggctgctgaggcattatcaattgcggtcattgaagattttatgccaattatttcgagggtgaaatctgtagtgtcctcgaatggaggtttgaaaggagaaatcggttatgcaaaagaacatgaagctgtttggtttaaaggaaagagattcataccaaatgtatgggctaacacacctggtgagcagcaaataaaacaactgaagcctgcaattgattcaaaaggcagaaaggttggggaggaatggtttacaacaagcaaagttgagaatgctttagccaggtaccatgaagcttgtgataatgcaagaaataaagttcttgagctgttgagaggcctttctagtgaattgcaggacaaaattaacatacttgtcttttgctcaacactgctcatcattgcaaaagcactttttggtcatgttagtgaggctcgaagaagaggttggatgcttcctactatatctcccttatcaaaggactgtgttgtggaggaaagttcaagtgcaatggatttagtaggactatttccttactggcttgatgttaatcaaggaaatgcaatattgaatgatgtccacatgcactctttatttgttcttactggcccaaatggtggtggtaaatctagcatgttgcgatcagtctgtgcagctgtgcttcttggaatatgtggcctgatggtaccttcaacttcagctgtaatcccacattttgattccattatgctgcatatgaaagcctatgatagcccagcagatgggaaaagttcatttcagattgaaatgtcggagatacgtgctttagtcagccgagctactgctaggagtcttgttctgattgatgaaatatgtagaggcacagaaactgcaaaaggaacatgtatagctggtagcatcattgaaagacttgataatgttggctgcctaggcatcatatcaactcacctgcatgggattttcgacctgcctctctcacttagcaacactgatttcaaagctatgggaactgaagtggtcgatggatgcattcatccaacatggaaactgattgatggcatatgtagagaaagccttgcttttcaaacagcaaggagggaaggcatgcctgacttgataatcaccagggctgaggagctatatttgagtatgagtacaaataacaagcagggagcatcagtggcgcacaatgagcctcctaatggcagccccagtgtaaatggcttggttgaggagcctgaatctctgaagaacagactagaaatgctgcctggtacctttgagccgctgcggaaggaagttgagagtgctgttactacgatgtgtaagaaaatactgtcggacctttacaacaaaagtagcatcccagaactggtcgaggtggtctgcgttgctgtaggtgctagagagcaaccaccgccttccactgttggcagatctagcatctacgtgattatcagaagcgacaacaggctctatgttggacagacggacgatcttctggggcgcttgaacgcccacagatcgaaggaaggcatgcgggacgctacggtattatacgtcttggtccctggcaagagcgttgcctgccagctggaaacccttctcataaaccagctcccttcgaggggcttcaagctcatcaacaaggcagacgggaagcacaggaacttcggtatatctcgaatctctggcgaggcagttgctactggacggaactagtcagctgaaggtctttttgctattggatattcaataggaggcgggagctaactgacaccatgccgccctagttttgttgaactgatagtggtgctagccttgtgcatagtaggggccaggggagtaataatatttcccttgtctaaggaagtaattaaggatgtaggagcgcagagcaaatgattagcaaaacgatcacgttcgcagtgcggcaggcattgtgcagctgattcgtttgttcaatcagtgctctcgctcagtgacctgacagcgtgatcactaaacctagtagctgggcgcgcctttctaggccagaacagaacattggcgtgaattagattccagtgcggcagggtgttccctaagtacatgttttcagttcagcggcacttggaatatttttatgagaaaaagctgcgtcttttagcttgga

SEQ ID NO: 10 - DNA, 2331 bp, Arabidopsis thaliana
  source 1..2331: mol_type = genomic DNA
gtttcctcctccagtaaactgacgacgatacaagctctctcaccaacggcgacagtatctttgccggcggcgggagcgcttctgcttaatctgtagctcaaactaaaaattctctgggtattactcatggtgatttggaataacgatgatgatgattttttggagattgataactttcaatcttctccacggtcatctccaatacatgcaatgcagtgtagggtcgaaaatctagctggtgtagccgtgacaactagttctttgagctctcctactgagacaactgatttagttcagatgggcttctcagacgaggtttttgctacattgtttgacatgggatttcctgttgagatgatttctagagcgatcaaggaaactggaccaaatgtagaaacttcggttataattgatactatctccaaatactcaagcgactgtgaagctggttcttccaagtccaaggctattgatcatttccttgctatgggatttgatgaagaaaaagttgtcaaagccattcaagaacatggagaagacaatatggaagcaattgcaaatgcattgctctcttgtccagaggctaagaaactgccagcagcagtagaggaagaagatggcattgactggtcatcaagtgatgatgataccaattacaccgatatgttaaactcagatgatgagaaagatccaaactcaaatgaaaatggcagcaaaatacggtctttggtgaagatgggtttctcagagcttgaagcttctttagctgtcgagagatgtggagaaaatgtggatattgcagagctcacagacttcctttgtgctgctcaaatggctagggaatttagtgagttttacactgaacatgaagaacaaaagcctagacataatattaagaaaaggcggtttgagtcaaaaggagagccaagatcatctgttgatgacgagccgattcgtctaccaaatccaatgataggatttggggttccaaacgagcccggactcattacacatagatcgcttccagagttagcccgagggccaccttttttctactatgagaatgtcgccctcacacctaaaggcgtttgggagactatttccaggcacttgttcgagatcccacctgagtttgtggactcaaaatatttctgtgttgcagcgaggaagagaggctacatccacaatctccccatcaacaacagatttcagattcagcctccaccaaaatacaccatccatgatgcatttcctttgagtaagagatggtggccagaatgggataaaaggaccaagcttaattgcattttgacttgtacaggtagtgctcagttgactaacaggattcgtgtagcccttgagccttacaatgaagaaccagaaccgcctaagcatgtacaaagatatgtgattgaccagtgcaaaaaatggaatttggtttgggtgggtaaaaacaaagctgccccactcgagccagatgagatggagagtattctgggatttccaaaaaatcatactcgtggtggaggcatgagtagaactgagcgcttcaagtccttaggaaattcgtttcaggttgatactgtggcgtatcatctgtctgtcctgaagcccattttcccacatggaatcaatgttctctctcttttcacgggtattggtggtggggaagtggcacttcatcgtctccaaatcaaaatgaagcttgttgtgtctgttgagatttcaaaagtcaacagaaatattttgaaggacttttgggagcaaactaaccagactggagaattgatcgagttttcagacatccaacacttgactaatgacacaatcgaagggttgatggagaaatatggtggatttgatcttgtaattggaggaagtccttgtaacaatctggcaggcggtaatagggtaagccgagttggtcttgaaggtgatcaatcttcgttgttctttgagtattgccgtattctagaggtggtacgtgcgaggatgagaggatcttgatcattcacattacctgaagtagattgaagtagatatgactccatgaaatctagttgaactttatttttctatatctgttatgtaatatggccatgaaggtacatttttgaataatataagtcaacctaactctcttttgctttttgcgacaatatatctgcaactgcatttgcacttttctcttccctgccttaacactgatgttcagtaatgtattcaaccagacaaatgtgtcatcatataatctttgatccttccttcatgcgaagggttaatggagaaatatgtttggggtatttgtatagacactttggttaaggagtc

SEQ ID NO: 11 - DNA, 1773 bp, Glycine max
  source 1..1773: mol_type = genomic DNA
atgggaggagatgattctggtttggagagtgacaattttgattggaacactgaagatgagcttgaaattcagaactataactcgtcgtcttcatgtttaacccttcctaatggagatgctgttactggctctggagaggcaagctcgtctgcagttttggctaattccaaggtgcttgatcacttcgtcagcatgggattttctagagaaatggtttctaaagtaattcaggaatatggtgaggaaaatgaagataaactacttgaagaacttctcacatacaaggctctagaaagttcttcccgtccacagcagcgaattgagccagatccttgttcttcagagaatgcagggagttcttgggatgatttctcagatactgatattttttctgatgatgaagaaattgcaaaaactatgtctgagaatgatgataccttacggtctttggtgaaaatggggtacaagcaggtggaggctttaattgccatagaaagattaggcccaaacgcctcacttgaagaattggtagattttataggtgttgctcaaatggcaaaggctgaagatgctcttctgcctcctcaagaaaagttacaatacaatgactatgctaagtccaataaacgaagattatatgattatgaagtgctgggaaggaaaaagcctaggggatgtgagaagaaaatcctcaatgaagatgacgaggatgctgaagctcttcatcttccaaatccgatgattgggtttggtgtacctacagagtcaagcttcataacacacagaaggcttccagaagatgccattgggcctccttacttctattacgagaatgtagccttagcaccaaaaggtgtttggcaaacaatttcaagattcttgtatgatgttgaacctgagtttgtagattcgaagtttttctgtgctgcagccaggaaaaggggatatattcacaatctccccatccaaaataggttcccacttctaccacttccaccgcgcacaatacatgaggcttttcccctaacaaagaaatggtggccctcatgggatattaggaccaagcttaattgtttgcaaacatgtattggcagtgcaaaactaacagaaagaattaggaaagctgtagaaatctatgatgaagatccacctgaaagtgtacagaagtatgttcttcatcagtgtaggaaatggaatttggtttgggtgggaagaaataaggttgctccattagagcctgatgaagtagaaacgctgttgggcttcccaaggaaccacaccagaggaggtgggataagtaggactgacagatacaagtcacttggtaattcattccaggttgacactgtggcataccacttgtcagttctgaaggagatgtatcctaatggtatcaatcttctttctctcttttctggaattggtggggcggaggtagctcttcatcggcttggcatccctctcaagaatgttgtgtctgttgaaaaatcggaagttaataggaatattgttagaagttggtgggagcaaacaaaccagaaaggtaatctatatgatatggatgatgtaagggagctagacggtgatcgcttggaacagctgatgagcacatttggtggttttgatctaattgttggtggcagtccatgtaataacctggctggtagcaacagggtcagccgggatggactggagggaaaagaatcttctctcttctttgattattttaggattctagacttggtaaaaaatatgtcggccaaatatcgatga

SEQ ID NO: 12 - DNA, 1743 bp, Glycine max
  source 1..1743: mol_type = genomic DNA
atgggaggagatgattttgattggaatactgatgatgagcttgaaattgagaactataactcttcatcttcgtgtttaacccttcctaatggggatgctggagaggcaagctcgtctgcagttttggctaattccaaggtacttgaccacttcgtcaacatgggattttctagagaaatggtttctaaagtaattcaggaatatggtgaagaaaatgaagataaactacttgaagaacttctctcatacaaggctctggaaagttctccccaaccacagcagcgaattgagccagatccttgttcttcagagaatgcagggagctcttgggatgatttctctgatactgatattttttctgatgatgaagaaattgcaaaaactatgtctgagaatgatgataccttacggtctttggtgaaaatgggctacaaacaagaggaagctttagttgccatagaaagattaggcccaaatgcctcacttgaagaattggtagattttataggtgttgctcaaatggcaaaggctgaggatgctcttctgcctcctgaagaaaagttacaatacaatgactatgctaagtccaataaacgaagattttatgattatgaagtgctgggaaggaaaaagcctaggggatgtgagaagaaaatcctcaatgaagatgatgatgaggaagatgaagctcttcatcttccaaacccaatgattgggtttggtgtacctacagagtcgagcttcataacacacagaaggattccagaagatgccattgggcctccttacttctattatgagaatgtagccttagcaccgaaaggtgtttggcaaacaatttcaagattcttgtatgatgttcaacccgagtttgtagattcaaagtttttctgtgctgcagccaggaaaaggggatatattcacaatctccccatccaaaataggttcccacttctaccacttccaccgcgcacaatacacgaggcttttcccctaacaaagaagtggtggccttcatgggatactaggaccaagcttaattgtttgcaaacatgtattggcagtgcaaaactaacagaaagaattaggaaagctgtagaaatctatgatgaagatccacctgaaagtgtacagaagtttgttcttcatcagtgtaggaaatggaatttggtttgggtgggaaggaataaggttgctccattagagcctgatgaagtagaaacgctgctgggcttcccaaggaaccacaccagaggaggtggaataagtaggactgacagatacaagtcacttggtaattcattccaggttgacactgtggcataccacttgtcagttctgaaggagatgtatcctaatggtatcaatcttctttctctcttttctggaattggtggggcagaggtagctcttcatcggctcggcatccctctcaagaatgttgtgtctgttgaaaaatcagaagtgaataggaatattgttagaagttggtgggagcaaacaaatcagaaaggtaatctatatgacattgacgatgtaagggagctagacggtgatcgcttggagcagctgatgagcacatttggtggttttgatctaattgttggtggcagtccatgtaataatctggctggtagcaacagggtcagccgggatggactggagggaaaagaatcttctctcttctttgattactttaggattctagacttagtaaaaaatatgtcagctaaatatcgataa

SEQ ID NO: 13 - DNA, 1963 bp, Brassica napus
  source 1..1963: mol_type = genomic DNA
atgcaggcgcaagattctgctgactggaacagcgatgatgatttggatttgcagattgataactaccagtcatctccaccgtcatctccaccgccggcggtcccttcttccgatgagctgtttgctacgctggttgacatggggttctctcctgagaatattgctagagcaatcgatgaacatggaccaaatgcagatactgtggtgatcattgatgctatctccaagtacactgttaactgtgaagctagttcttccaagtccaagactattgatcatttccttgctatgggatttgaagaagagaaagttatcaaagccattcatgagcatggagaagacaatattgaagagatagcaaatgcactcatctcctcggcagcagatgcatcgcctgtaatcaaagaagaagaggacattgagtggtctgagagtgatgatgagaagtatgccaattttctcagctcagatgatgagaaagtttcagactcctcctctgaaaacggcaatcaattgcggtctttgatgaacatgggattctcggagttggaagcttctcttgctatacagagatgtggtactggagaggatgtgagcattgcagatctcgcagacttcatttgtgccgctcagattgctcgagagttcgatgagttccatgctgaccctgaagaacaaaagcctaatgttaaaaagaggcgtatagagccaagaagagagccgaggtcatctgctgctgatgacgagccccttcgtctgccaaatccgatgattggattcggcgttccaaacgagccaggactcatcgcgcagagatctcttccagccatggctcgaatgcctccatttttctactacgagaacgtcgcccttgctcccaaaggcgtctgggacaccatttctagccacttgtacgacgttccaccggagtttgtggattcgaaatacatttgtgcagcagcaaggaagagaggatacgtgcacaacctccccatcagtggaaggtatcaggtccagcctcctccgaagtacacgatcagcgaggcgtttccgctgaccaagaagtggtggccagagtgggacaagagaacaaagctcaactgcatcctgacagtgaacgctagtgctcagctgacgaacaggatccggttagcacttgagccttatagtggagaaccgcctgagagtaagaagaagtatgtcattgagcagtgcaagaagtggaatcttgtgtgggtgggaaagaacaaagctgcgccacttgaaccagatgaaatggagactcttcttggatttccaagaaaccatacacgaggcggtggcatgagtaggaccgagcgtctgaagtcactcggcaactcttttcaggtggataccgttgcgtatcacctctctgttttgaaaccattgtttccatatggtatcaacgttctgtccctgttcactggaattggcggtggagaagtggcgctccaccgtctccagattccaatgaagacggtcgtttcggtggagatctcagcagggaacagaaacattctcaaggatttctgggagcaaaccaaccagaaaggaaccctgatagagtttgcagacgtgcaagacctgagtaacgagaagatcgtggagttgatgaagcagtttggtgggtttgatcttgtgatcggaggtagcccctgcaacaacctcgctggtggtaacagagtgagcaggagtggtcttgaaggtgatcagtcctgtttgttctatgagtattgtcggattctcgatgtggtgcgagagacaacaaagacgatgagaagatcctaggcggtgggaggttgagatagtttcttataacataagtaacttcgtgcagacaatctgtgaagtacttcttgtttggttcttactttgtagaacttgaatgtagactctcatttggatgattctgcaaagctttgaggataactcagctttaaat

SEQ ID NO: 14 - DNA, 1956 bp, Brassica napus
  source 1..1956: mol_type = genomic DNA
atgcaggcgcaagattctgctgactggaacagcgatgatgatttggatttgcagattgataactaccagtcatctccaccgtcatccccaccggcggcagtcccttcttccgatgagctgtttgctacgctggttgacatggggttctctcctgagaatattgctagagctatcgatgaatatggaccaaatgcagatactgtggtgattattgatgctatctccaaatacactgttacctgtgaagctagttcttccaagtccaagactattgatcatttccttgctatgggatttgaggaggagaaagttatcaaggctattcatgagcatggagaagacaacattgaagagatagcaaatgcactcatctcctcggcagcagaggtatcgcccaaaatcaaagaagaagaggacattgagtggtctgagagtgatgatgagaagtatgccaatttcctcagctcagatgatgagaaagtttcagactcctcctctgaaaacggcaatcaattgcggtctttaatgaacatgggattctcggagttggaagcttctcttgctatagagagatgtggtactggagaggatgtgagcattgcagatctcgcagacttcatttgtgccgctcagattgctcgagagttcgatgagttccatgctgaccctgaagaacaaaagcctaatgtcaaaaagagacggatagagccaagaagagagccgaggtcatctgctgctgatgatgagccccttcgtctgccaaatccgatgattggatttggcgttccaaacgagccaggactcatcacgcagagatctcttccagccatggctcgaatgcctccatttttctactacgagaacgtcgcccttgctcccaaaggcgtgtgggacaccatttctagtcacttgtacgacgttccaccggagtttgtggattcgaaatatatttgtgcagcagcgaggaaacgaggatacgtgcacaacctccccatcagtggaaggtatcaggtccagcctcctccgaagtacacgatcagcgaggcgtttccgctgaccaagaagtggtggccagagtgggacaagagaacaaagctcaactgcatcctgacagtgaacgctagtgctcagctgacgaacaggatccggttagcacttgagccttatggtggagaaccgcctgagagtaagaagaagtatgtcattgagcagtgcaagaagtggaatctggtgtgggtgggaaagaacaaagctgcgccgcttgaaccagatgaaatggagactcttcttggatttccaaaaaaccatacacgaggcggtggcatgagtaggaccgagcgtctgaagtcactcggcaactcttttcaggtggataccgttgcgtatcacctctctgtcttgaaaccattgtttccatatggtatcaacgttctgtccctgttcactggaattggcggtggagaagtggcgctccaccgtctccagattccaatgaagaccgtcgtttcggtggagatctcagcagtgaacagaaacattctcaaggacttctgggagcaaaccaaccagaaaggaaccctgatagagtttgcagacgtgcaagacctgagtaacgagaagatcgtggagttgatgaagcagtttggtgggtttgatctagtgatcggaggtagcccctgcaacaacctcgctggtggtaacagagtgagcaggagtggtcttgaaggtgatcagtcctgtttgttctatgagtattgtcggattctcgatgtggtgcgagaaacaacaaagacgatgagaagatcctaggcagtgggtggttgagatagtttcttataacataagtaactttgtgcagacaatctgtgtcttgtttggttcttactttgtagaactcgaatgtagtgactctcatttggattctgctaagctttgaggataactcagctttaaata

SEQ ID NO: 15 - DNA, 1830 bp, Solanum lycopersicum
  source 1..1830: mol_type = genomic DNA
atggacaaaaatctttctggagaagataatgatgacattgactgggatactgaagatgaacttgaaatacaagaaatacaggacacagtgttttcctcatgcacggatttaagaactactgggcagcatgttgtttgttgcgatgtggaggcaagctcgtcatcagtacccttccggtccaagttcattcagcagtttgtagtgatgggatttcctgaagaatctattgcaaaagcaatagagcaaaacggagaaaattcagatttggtgctggattctcttttgacttttaaggcccttgatgactctcctgaagaacagcccagtgttagccctccgctggaaccctccattagttctgatgacagcgcttctgaatacaacaagattgttttggataatgtttatgaggatgatagttggtcttctgactcagacaactacataaatactgttaagcagtgctacttgaatgacgagggaagttctttgtctgaaaaagaaaagatgttattgttcctgggaaatatgggatatccagcggaagaggcctccatagcaatggatagatgtggtccaaaagcatcactccctgaattggtagattttatttgtgctgctcaaatgtcaagagcggaagatccctatctcctagaagatgtgaagccaaacctgaaggatatattgaatgattgtggtggatacaaaaagaggaaaatgtacaatgaattgtgcaaacggaaaaagcaaagggagatttctgttgaagagccaattcgattgcccaagccgatgattggctttggaattcctaccgaatcagttccaagaatggttcaacgaattcttcccgagaaaatcattggcccgccttatttctattatgaaaatgtcgccctggctccaaagggtgtgtgggacaccattaagaggcatttgtatgagattgagcccgagtttgttgactcgaagtacttttctgcaactgcaagaaaaagagggtatcttcataatctgccgattgaaaacagatttcctttgtttccacttcccccacggaccattcatgaggcacttcccctttcgaagagatggtggccatcttgggatactagatcaaagttgaactgcttacaaacagccattgggagtgcaaggttgacggataagatcaggaaagctgtcgagaagtatgatggtgaaccacctatggaaatacagaagtatgtactctatcattgcaagaagtggaatttggtctgggtaggaagaaacaaagttgcccctttggaacctgatgaagttgaaatgctattggggttcccaaagaatcacacaaggggaggaggtataagtaggacggacagatacaagtcactcggtaactctttccaggttgacacagtggcgtaccacttatcagtgttgagagacttgtttccaaatggaatcaatgtcttatcactcttctctggcattggtggtgctgaagttgctctctaccgtctcggtgttcccctcaacaatgttgtttcagttgaaaagtctgaagtgaacaggaatattgtgagaagctggtgggagcaaacaaatcagagggggaatctcatacattttgacgatgtgcagctgctgagcagagatcggttgaagaagttaatagaatcagttggtggatttgatttagtgattggtggaagcccatgcaacaaccttgcaggcagtaacagggtgagcagggatgggcttgaaggcaaagagtcttctctgttttttgattatgttcggatattggatgatgtcaagtccataatgtctagacatagatga

SEQ ID NO: 16 - DNA, 2090 bp, Solanum lycopersicum
  source 1..2090: mol_type = genomic DNA
gagttcggagttctttttaaggaattacctttaaaaatggataacaacttttctggagaagacaatgacagcattgactgggatactgaagatgaattagaaatacaagaaatgccggatgcaacattttcctcgtgcaccaatttaagaagtgttggatatcataccgttagtggtcatagggaggcaagatcatcatcagaaccatgtcaatctaagttcattcaacaatttatcgtgatgggatttcctgaagagtctattgcaaaagcgatagagcaaaatggagaaaatgaaggtttggtgctagatgctcttttgacattcaaggcacttgaagattctcctgaagaacagccgagtacgagcactcagatggaaccctgcattacttctgatgatagctcttcccagtacaacgaaaactttttggatgatgtttctgaagatgatagctggtccttggactcggacaattgtgtaaattctgctaaacagagctacttgaatgacgacaactgttctttgtctgaaaacgagaagacattattattcctagcaaatatgggataccctgcggaggaggcttccatcgcaatggagagatgtggtccagaagcaccgtttccggagttgatagatttcatgtgtgctgctcaaatggcaagagaagaagacgtccatctgcccgaagatgaaaagccaaaactaaatagtggtggatacaaaaggaagatgtataatgaagttcgcgtaaagaaaaagcaaagggcgataactgatgaagagacaattcatttgcccagacctatgattggattcggggttcctacggaatccttaccagctgttgtgaaaagaactctgccagagcaagctattggccccccttttttctattatgaaaatgttgctcttgctccgaagggtgtgtgggacactatgacaagattcctttacgatattgagcccgagtttgttgactcgaaatatttttgcgctaccgcaaggaaaagaggatatattcataatctaccaattgaagatagatttcctttacttccacttcctccacgtaccattcacgaggcgtttcctctaacgaagaaatggtggccatcttgggatacacgaacaaagttaaattgcttacaaacatctattgggagtgcaaggttagctgatagaattaggaaagctatgaaagcgatggagaacttcgatagtgagccaccgttgatggtgcaaaagtatgttctagacgaatgccggaaatggaatttgctgtgggttggtagaaataaagtctctcctttggagactgatgaatttgaaatgctaatgggatttccacggaatcatacccgtggaggaggtataagtaggaccgatagatacaagtcgcttggtaactcgttccaggtcgacacagtggcataccatttatcggtgttgaaagacttgtatccaaatggtataaatgttttatcactattttctggaattggcggtgctgaagttgctctgtatcgtctcggtattccattgaacaatgtggttgccgtagagatatctgaagttaacaggaatattctgagaagctggtgggagcaaacaaatcaaaaggggaatcttatagatttccatgatatccagcagctgaacggagacgtcttggagcaaatgatcgattcaattggaggatttgatttagtaattggcggaagcccctgcaacaacctcactggaagtaacagggtgactagagacggactcgaaggtaaagattcttctctattttatgactatgttcgggtagtggacttggtcaaatccattatgtctaatcgtagagtgtgacgaaaatatgttttgagtttctgaacttggaaggcaacaagtaaaaagttgtctttgattttggtttgtacatatttttcgtaaaagggggcacaaagtaactctcttagtgccatcctaatataactgttgaacgttcgtggaaagaatgaactgttgaacttctcattgtaaacgacattaaatgatgttgaaaatgaacttgtgattttcgcttagctcatgggcaa

SEQ ID NO: 17 - DNA, 2132 bp, Solanum lycopersicum
  source 1..2132: mol_type = genomic DNA
atggacaaacatctttctgaagaagatagtgacaacattgactgggatactgaagatgagttagaaatacaggatacaacattttcctcatgcagggatttaagaactaatggacagtatgctattagtggtgatggggaggcaagctcatcatcagtacctggccagtctacgttcattcagaagtttttagtgatgggattttctgaagagtccattgcaaaagcaatagagcaaaatggagaaaattcagatttggtgctggatgctcttttgacattaaaggccatcgaagattctcctgaagaacagcccagtgctagccctcacctggagccctgcattaattctgatgatagctcttctgaatacaatgagaactttctggatgatgtttatgatgaagatagttggtcctctgactcagactactgtacaaattctgttaaacagtgctatgtgaaagaagagagcaattctttgtctgaaaaagagcagacaatcttatttctggcaaatatgggatacccagtggaagaggtttctatagcaatggagagatgtggtccagaagcatcggtttctgaattgacagatttcatttgtgctgctcaaatggcaagagaagaagatccctatttgccagaagatgtaaagccaaaactgaaccatggtagtggtggatacaagaagaggaagatgttcaatcagttgtgcaaaagcaaaaagccaagggcgatttttgatgaagagacaattcgtttgcccaaacctatgattggatttggggttcctacagaatcagtttctgcaattgtccgaagaactattccggagcaagcttttggccccccttttttctattacgaaaatgttgctctagctccaaagggtgtgtgggataccatttccagattcctatatgatattgagcctgagtttgtcgattcaaagtatttttgtgctactgcaagaaaaaggggttatattcataacctacctactgaaaatagatttcccttgcttccacttcctccacgcaccattaatgaggcacttccccttacaaagaaatggtggccatcttgggatccacggacaaagttaaattgtttgcaaacagctattggaagtgcaagattgactgataggatcaggaaagctgtggaggcctttgacggtgagccacctatgagggtgcaaaagtttgttcttgatcaatgtcggaagtggaatttggtgtgggttgggagaaacaaagttgctcctttggagccagatgaatttgaaatgctattggggtttccaaagaaccataccaggggaggtggtataagtaggaccgatagatacaaatcgcttggtaactcattccaggttgacacagtggcataccatttatcagtgttgaaagacatgtttccaaatgggatgaacgtcttatcactcttctctgggattggcggtgctgaagttgctctttaccgtcttgggattcaactaaacaatgtggtctctgtggaaaaatctgaagtgaacagaaacattgtgagaagttggtgggagcaaactaatcagagaggcaatcttattgattttgatgatgtgcagcagttgaatggagaccgcttagaacaactgatagattcatgtggcggatttgatttactgattggtggaagcccgtgcaacaatcttgcaggcagtaacagggtgagcagggacggtcttgaaggcaaagaatcgtctctattttatgactatgttcggatactggacttagtcaagtccataatgtctagacagagatagtatgacagaaattcgttctagcttcagattcttagttatctttcaaaattatgaaacattacattaagggataaagccatcacataaaatttctggcttaagattctacatcgactggtctcctacaataccagttgatgagcattctatttcgctttgaagcgaaaacaaacatgaagtgtaaagtacattgttactattagttattggcctaacatgccatttcagttgatgagcatgcacagttacagtgttgtatcctccatgaataatattctggaatttgcacaagaaagctctgcacttgaatctcaatgtta

SEQ ID NO: 18 - DNA, 1794 bp, Oryza sativa
  source 1..1794: mol_type = genomic DNA
atggtggactgggcttcagatagcgacaatgataagttcgagtgggacacagacggtgaggctgagacctcatctgccccggccctaaggaacatagatgctcctggtccgtccacgaggctgccgcaggatgcaaatgggaaggccaacgggtcaggtgctttggttgctgaatttatgggaatgggattcccaaaagaaatgatcttgaaagcgattaaggagattggggatacagatacagagcaattgcttgagttacttctgacttatcaggcaataggcggtgacgcttcagtgggtaactgctctgcttcggcctgtgccccccagactcttgaagtcgacgaggaggaggatgatactaattgggatgaatatgatactgctggcaattgtgacaggactcctcactctgatggttctggtgacgaggatttctttcaagaaatgtcagagaaggatgaaaaaatgaagtccttagtcaacatgggttttcctgaagatgaagcaaagatggctatcgatagatgtctcgatgcgcctgtagcagtgttggttgattcaatctatgcatcacaggaagcaggaaatggttactctgcaaacttatctgactatgaggatacagagttcagttcctttggaggaagaaagaaaacaagatttgtggatggaagcaagaagagaaagcggtatggaagtgggccatctgggaatcaagtgccgtttgatggcagccacgaagagcccatgcctcttccaaatccaatggtgggattcagcttgcccaatgagaggttaaggtcagtccacagaaatcttcctgaccaagctcttgggccaccattcttttactatgaaaacgtggccctagctccaaaaggtgtatggacaaccatctcaaggtttttatatgacattcagcctgagtttgtggactccaagtacttttgtgctgctgccaggaagaggggttatattcataacctgccgattgagaacaggtcacctgttctcccaatgcctcccaaaacaatatctgaagcctttcctaataccaagaggtggtggccttcctgggatccaaggaggcagttcaactgcttgcaaacttgtatggcaagcgcaaagcttacagagcgaattcgttgtgctttgggtagattcagtgatgtaccaactccacaagttcagaagtatgtcttggacgaatgtaggaaatggaacctagtttgggtcggaaagaataaggtcgctcctctagagcctgatgagatggagttcctgttagggtaccctagaaaccacactaggggagttagcaggacggagaggtacagggctctcgggaattcattccaagttgatacagtagcataccatctctctgtgctgagggacctgtttcccaatggaatgaatgtcttatccctgttttctggtattggaggagcagaggtagctctccacagactggggatacatatgaaaacagtgatctctgttgagaaatcagaagtgaacagaacgattctgaagagctggtgggatcagacacaaactggaacgctgattgaaatcgccgacgtgcggcatcttactactgaaagaattgaaacattcatcagaaggtttggcggttttgacctggtgattggtggcagcccgtgcaacaaccttgctggcagcaaccgccatcaccgagatggtctggagggcgagcactccgcgctcttctacgattacattagaatattagaacatgtaaaggctaccatgtcagcagtctag

SEQ ID NO: 19 - DNA, 2374 bp, Sorghum bicolor
  source 1..2374: mol_type = genomic DNA
gtgcccattgcccgtgctctttggcggacgcgtcgcgtgacggcgtgagtttctcggagtcctggagattcgtcgccgtcgcggcctcctgctcctcctcgtctgccactggcttccaagtcccagcgcgcaggggccgacggcgacgggtggggactggggagaggcggcagaggtaatccgggcaagctgtggagcggagagggcggagagaggctgcgccgggccacgccgtccgggggtccgtcgccgcagcttccggccacgccggggccgtggaaaacgagagggatcgagggaggggtatccgtcggagcccctttgccaaggttccggaatggcgcactgtgttggcgacagtgatggtagtgacaacttcgaatgggatagtgacggcgatggggaggaggcagcgagcttcaacgctgctggtgccagttcatcagcaatggcatcaacgaacgctgatgctccaggcccatctacacgggttgctaatggcaatgggaaggctgggccatctgcctctttggttcataagtatatggacatgggtttcccagaagagattgttctgaaggccatgaaggacaatggggataatggagcagattcattagttgagcttcttcttacttaccaggaactaggcaatgacctcaaagtggataatggctttgcttctggttgtgtcccccaaaatgttgacgatagtgatgatgatgacattctagaaaactgggatgatgaggatgctggtggtagaagcaccggggttgctaactcggttgatgattctggagatgaggatttcttacatgagatgtcacagacggacgagaaagtttattccttagttaaaatggggtttcctgaagacgaggctgcactggctgttactagatgcgggcaggatgcatctatttcggttctggtggattcaatatatgcttcacagactgcaggagatgtttactgtggcaatttgtctgactatgaggataattcctacggagggagaaacaaaggaaggttcatggatgcgaacaaaaaaaggagaaagagatatgaaggtcaagcacacggaagtagaggcccattggatggtagctctgatgaacccatgcctctcccacatccaatggttggatttagtttgccacaccagtgcacaaggccagtggacagatcactgccttcacaagctatgggtccaccatacttctactatgagaacgttgctcttgctccaaaaggtgtttggactaccatatcaagattcttgtatgatattcagccagagtttgtggactcgaagtacttctgtgctgctgccaggaaaagaggttatatacacaatctgccacttgagaacaggtcacctctcctccccatacctccaaagaaaatatcagaagcatttcctcataccaagaggtggtggccatcatgggacccaagacgacagttcaattgcctccagacttgtgtgtctagtgcaaaactgttagagaggattcgtgtagctcttacaaacagttcagatccacctcctccaagagttcagaagtttgtgttggaggagtgtaggaaatggaatctcgcttgggttggcttaaacaaggttgctcctctagagcctgatgagatggagtttctactcggttttccgaaggatcacaccagaggtatcagcaggagagagaggtaccgatctctaggaaattcatttcaagttgatactgttgcttaccatctctcggtcctcaaggatatgtacccacaaggcatgaatgtactgtctttattctctggtattggaggagcagaggtggctctccacaggcttggtatacgcatgaaaacagttatttcagtagagaagtctgaggtcaataggacgattctgaagagttggtgggatcagacgcagactggtcttttaattgagatttgtgatgtgcagacattgacatctgagaggatcgaggcatatgttagaagaattggtggctttgatcttgtgattggtggaagtccctgtaacaatcttactgggagcaaccgttaccacagagatggtttggagggcgagcattcttcgttattctaccactatgttaggatcttagactctgtcaagtccatcatggagcgtttgtagtttttagaatctttattattttggttctagaattgtagcatcttttcttgtgatcaaactggtgaatgatccgaaggttgactggtgtagttctcatgccactctaatttaggctacttttaatttaatctcgtttggtgatcaatgcaaattgatgttggtgcagttgtgtggcactggcagttctgttgctttccctttttaagtttg

SEQ ID NO: 20 - DNA, 2400 bp, Zea mays
  source 1..2400: mol_type = genomic DNA
ctaaagcggtcagtgggtggaggcagccaagagtcaccctgtcgccgttgccgtcgccgaggcaggcagagtctccctgtcgccgttgccgtcgccggcctcctcctcctctgccacttcctcccaactcccagccgcaggggccgacggcgacgggagggaagaggcggcagaggtactccgaggggcgcggagagaggctgtgccaggccacgccgtccagtgcctccgggtccgtcgccgcagcttccggccgcgtcggagaggtagcccccggagctcttcgcggaggctcgggaatggtgcactgggttagcgacagtgatggcagtgataacttcgaatgggacagtgatggtaacggggagcagacagtgagcttcaacgctgctggtgctggttcatcagctctggcagcgacgaacactgatgctcctggcccatcgacacgggttgctaatggcaatgggaaggctgggcgatctgcctctttggttcagaagtatgtggacatgggtttctcagaagagattgttctgaaggccatgaaggacaatggggataatggagcagattcattagttgagctccttcttacttaccaggaactaggcaatgacctcaaagtggataatgactttgcttctagttgtgcccccaaaactgctgacgatagtgatgatgatgacacactggaaatctgggatgatgaggatgctggagggagaagcaccagggttgctaactctgttgatgattctgatgacgaggatttcttacatgagatgtcacggaaggacgaaaaagttgattccttagttaaaatggggtttcctgaagacgaggctgcactggctattaccagatgcgggccggatgcatctatttctgttctggtggattcaatctatgcttcacagaccgcaggagatggttactgtggcaatctgtctgactatgaggataattcctatggagggagaagcacagggaacaagaaaaagagaaaaagatatggaggccaagcacagggaagtagaggcccattagatggtagctgtgatgaacccatgcctctcccacatccaatggttggatttaacttgccagaccagtggtcaagacgagtggacagatcgttgcctgcacaagctattggtccgccgtacttctactacgagaacgttgctcttgctccaaaaggtgtctggactaccatatcaagattcttgtatgatattcaaccagagtttgtggactcaaagtacttctgtgctgctgccaggaaaaggggttacatacacaacctgccacttgagaacaggtcacctctcctccccatacccccaaagacgatatcggaagcatttcctcggaccaagaggtggtggccttcatgggacccaagacgacagttcaattgcctccagacttgcgtgtctagtgcaaaattgttagagaggattcgcgtagccctcacaaacagttcagacccacctcctccaagagttcagaagtatgtgttggaggagtgtaggaaatggaacctggcatgggttggcttaaacaaggttgctcctctagagcctgacgagatggagtttctactcggctttccgaaggatcacacgagaggtatcagcaggacagagaggtatcgatctctaggaaattcatttcaggtcgatactgttgcttaccatctctcagttctgaaggatctgttcccacaaggcatgaatgtgctgtctttattctctggtattggaggagcagaggtggctctccacaggcttggcatacagatgaacacggttatttcagtggagaagtccgaggtcaaccggacgattctgaagagttggtgggatcagacgcagacgggtactctgattgagatcactgatgtgcagacactgtcatctgagaggatcgaggcgtatattagaagaattgggggcttcgatcttgtgattggtggaagtccctgtaacaacctcactgggagcaaccgtcaccacagagatggtttggagggcgagcattctgcattgttccatcattattttaggatcttacacgccgtcaagtccatcatggagcgtttgtagttcttagaatctttactgttgttttggttctagtagaatactttgtgaccgaacttgtaaatggtttgcaggttgactggtttagttctcttgccactctaatttaggctagttttttttttatttaatcttctttggtgatcttggtgcagttctgtggcactgttgggtattcaaatgcagattgatccgagaggctgccctgttgcattttctttttaaaatttgtatttgtcaatgagagatcaggtattctggttgccaaaaaaaaatctgaagttgtggagct

SEQ ID NO: 21 - DNA, 2542 bp, Zea mays
  source 1..2542: mol_type = genomic DNA
cactgatcagccgccacacgggatctttcaacccctacggcctacgcgtacctgggaccgttcagcaccgtctcgctgacagacatgacacacatgaggcggtccctacaaatgctagtggtgtacaacaagtgacgtgcccaatgcccgtgcagtttggcgggcgccgcgcgtatgtttctcgttgcgacttggaggagaaggagattcgccgtctcaccccttccctgcctgctccctcgtctaagaaaagcgggcagagcagagacggcggcggcaggccatcgcggactcccgctcaccctcgtctgccaccgcctcccaggtcaggggccgacggcgacgggtggggaggggcggcagaggtagtccgaggggaggctgtggagcggagggcggagagaggccacgccgtccggagtctccagttccgtcgccacagcttcctgccgcgcctgggccgaggtaaacgagagggatcgaggaagaagtatcccccggagccccttcccccgaacccgaaggctccggaatggcgcactgggttagcgacagtgatggcagtgacaactttgaatgggatagtgatggcaatggggaggagccagcgaacttcaatgctgctggtgccggttcatcagctctgagatcaacgaacactgatgctccaggtccatcaacacgggttgctaatggcaatgggaaggctgggccatctgcctccttggttcaaaagtatatggacatgggtttcacggaagagattgttcggaaggccatgaaggacaatggggataatggagcagattcattagttgagcttcttcttacttaccaggaactaggcaatgacctcaaagtggataacggcttcgcttctggttgtgccccccaaactgttgatgatagtgatgatgacgacattctagaaaactgggatgatgaggatgctgatggtgggagaagcaccagggttgctaactctattgatgattctgaagacgaggtaattgcaatgctagtttatgtagcttgtcaacacaggatttctcagtgctctcctgtgctttcaacacaggatttcttaaatgagatgtcacagaaggacaagaaagttgattccttagttaaaatggggtttcctgaagacgaggctgcactggctattactagatgcgggcaggatgcatctatttctgttctggtggattccatatatgcttcacagaccgcaggagatggttactgtggcaatttgtctgactacgagggtaattccaatggagggagaaacaaaggaaggttcatggacgggaacaaaaaaaagagaaaaagatatggaggtcaagcacagggaaatagaggcccattagatggtagctgtgaagaacccatgcctctcccaaatccaatggttggatttaatttgccggaccattggacaagaccagtagacagatcgctgcctacacaagctattggaccaccatacttctactacgagaatgttgctcttgctccaaagggtgtctggactaccatatctagattcttgtacgatattcagccggagtttgtggactccaagtacttctgcgctgctgccaggaaaagaggctacatacacaatctaccacttgagagaaggtcgcctctcctccccttacctccgaagacaatatttgaagcatttcctcgtaccaagaggtggtggccttcatgggacccaaggcggcagttcaattgcctccagacttgtacgtctagcgcaaaactgttagagaggattcgtgtaacccttgcaaacagttcggatccaccacctccattagttcagaagtttgtgctggaggagtgtaggaaatggaatctggcttgggttggcctaaacaaggttgctcccctagagcctgatgagatggagttcctgcttggctttcccaaggatcacaccagaggtatcagcaggacagagaggtacccacaaggcatgaacgtgttgtctctattcactggcatcggaggagccgaggtggctctccataggctcggtatacgcatgaacacagtcgtttcagtagagaagtctgaggtgaacaggactattctgaagagctggtgggatcagacacagactggtaccttgattgagatcaatgatgtgcagacgctgacagctgataggatcgaggcatatattagacgaattggcggctttgatctcgtgataggtggaagcccatgcaacaatctcgctgggagcaaccgccaccacagagatggcttggagggcgaacattcttcattgttttaccattattttaggatcttggactccgtcaagtccatcatggagcgcctgtagtttttagaaacttttgatgttctggttcgtagaattgtagccgtctttccctgtgatcaaacttgtgaatgatttgaaggtcgactggtgtaatattcatgtcactctaatttaggctactttcgttaattacctcttgtgatcaatgcaaattgaaaggcaggtttcatttcttaa

SEQ ID NO: 22 - DNA, 19 bp, Synthetic construct
  source 1..19: mol_type = genomic DNA
gcaggccactgcacgtaga

SEQ ID NO: 23 - DNA, 19 bp, Synthetic construct
  source 1..19: mol_type = genomic DNA
cggtggaacgtcgtacaag

SEQ ID NO: 24 - DNA, 19 bp, Synthetic construct
  source 1..19: mol_type = genomic DNA
acagaacgttgataccata

(19) United States
(12) Patent Application Publication
(10) Pub. No.: US 2026/0297602 A1
(43) Pub. Date: Oct. 1, 2026

(54) INFLUENCE OF RdDM PATHWAY VARIANTS ON MSH1 GRAFT OUTCOMES

(71) Applicant: THE PENN STATE RESEARCH FOUNDATION, University Park, PA (US)

(72) Inventors: Hardik Kundariya, University Park, PA (US); Sally Mackenzie, St. Louis, MO (US)

(21) Appl. No.: 19/477,732

(22) PCT Filed: Apr. 26, 2024
(86) PCT No.: PCT/US2024/026457

§ 371 Date: Oct. 22, 2025

Related U.S. Application Data
(60) Provisional application No. 63/498,682, filed on Apr. 27, 2023.

(51) Int. Cl. C12N 15/82

(52) U.S. Cl. CPC C12N 15/8261

(57) ABSTRACT

The present invention provides methods for obtaining plants that exhibit useful traits by suppressing MSH1 and DRM2 gene expression in plant rootstocks and grafting the rootstocks to scions. Methods for identifying genetic loci that provide for useful traits in plants and plants produced with those loci are also provided. In addition, plants that exhibit the useful traits, parts of the plants including seeds, and products of the plants are provided as well as methods of using the plants. Recombinant DNA vectors and transgenic plants comprising those vectors that provide for MSH1 and DRM2 gene suppression are also provided.

### CROSS-REFERENCE TO RELATED APPLICATIONS

This International application claims benefit of provisional application U.S. Ser. No. 63/498,682, filed Apr. 27, 2023, which is hereby incorporated herein by reference in its entirety.

### INCORPORATION OF SEQUENCE LISTING

The instant application contains a Sequence Listing which has been submitted electronically in XML file format and is hereby incorporated by reference in its entirety. The XML file, created on Apr. 24, 2024, is named P13989WO00.xml and is 78,790 bytes in size.

### BACKGROUND

Evidence exists in support of a link between environmental sensing and epigenetic changes in both plants and animals (Bonasio et al., Science 330, 612, 2010). Trans-generational heritability of these changes remains a subject of active investigation (Youngson et al. Annu. Rev. Genom. Human Genet. 9, 233, 2008). Previous studies have shown that altered methylation patterns are highly heritable over multiple generations and can be incorporated into a quantitative analysis of variation (Vaughn et al. 2007; Zhang et al. 2008; Johannes et al. 2009). Earlier studies of methylation changes in Arabidopsis suggest amenability of the epigenome to recurrent selection and also suggest that it is feasible to establish new and stable epigenetic states (F. Johannes et al. PLOS Genet. 5, e1000530 (2009); F. Roux et al. Genetics 188, 1015 (2011). Manipulation of the Arabidopsis met1 and ddmt mutants has allowed the creation of epigenetic recombinant inbred lines (epi-RIL) populations that show both heritability of novel methylation patterning and epiallelic segregation, underscoring the likely influence of epigenomic variation in plant adaptation (F. Roux et al. Genetics 188, 1015 (2011)). In natural populations, a large proportion of the epiallelic variation detected in Arabidopsis is found as CpG methylation within gene-rich regions of the genome (C. Becker et al. Nature 480, 245 (2011), R. J. Schmitz et al. Science 334, 369 (2011).

Induction of traits that exhibit cytoplasmic inheritance (Redei Mutat. Res. 18, 149-162, 1973; Sandhu et al. Proc Natl Acad Sci USA. 104:1766-70, 2007) or that exhibit nuclear inheritance by suppression of the MSH1 gene has also been reported (WO 2012/151254; Xu et al. Plant Physiol. Vol. 159:711-720, 2012). The msh1 system in plants was initially developed in the model plant Arabidopsis, where msh1 mutation or RNAi suppression gives rise to a range in plant phenotype variation that is associated with sustained stress response (Xu et al. 2012; Shao et al. 2017) and epigenetic reprogramming of the plant (Virdi et al. 2015). Suppression of MSH1 expression induces, in approximately 20% of progeny in the next generation, a heritable, fully penetrant memory state that similarly displays sustained stress response and evidence of plant abiotic stress tolerance (Yang et al. 2020; Kundariya et al. 2022). Plants derived from grafting experiments that incorporate msh1 as rootstock and isogenic wild type as scion produce progeny with heritable enhanced growth vigor, resilience and seed yield (Kundariya et al, 2020).

These manipulations of the epigenetic msh1 state have been successfully recapitulated in soybean (Kechanmane Raju et al. 2018) and tomato (Yang et al. 2015; Kundariya et al. 2020), again yielding stress memory from MSH1 suppression and enhanced growth following grafting. However, graft experiments produce variable outcomes, with some proportion of graft progeny outperforming wild type but some grafts producing progeny with much smaller effect. These observations suggest that individual msh1 mutant rootstocks vary in the strength of their epigenetic “signal”.

Genetic experiments have demonstrated that msh1 epigenetic phenomena are at least partially dependent on the RNA-directed DNA methylation (RdDM) pathway (Yang et al. 2020; Kundariya et al. 2020), so that graft transmission of the msh1-derived growth effects requires sRNA production and transmission by the rootstock (Kundariya et al. 2020; 2022).

### SUMMARY

Grafted plants comprising a scion to which a rootstock had been grafted, wherein: (i) the scion is from a wild type plant; (ii) MSH1 and DRM2 gene expression is suppressed in the rootstock; (iii) the rootstock confers an improvement in yield or growth rate in progeny of the grafted plant in comparison to a control plant, wherein the control plant comprises either: (a) progeny of a scion grafted to rootstock that had not been subjected to suppression of MSH1 and DRM2 gene expression; (b) a whole plant that lacks any root graft and that had not been subjected to suppression of MSH1 and DRM2 gene expression; (c) a wild-type plant; or (d) progeny of a plant that is isogenic to the plant source of the scion of the grafted plant, are provided.

Selected populations of progeny plants produced by a method comprising the steps of: (a) obtaining a population of progeny plants from the aforementioned grafted plant, wherein the population of progeny plants is a first, second, or third generation of progeny plants obtained by selfing the grafted plant or by selfing the first or second generation progeny plants; (b) screening the population of progeny plants for improved yield or growth rate in comparison to a control plant population; and (c) selecting a population of progeny plants for an improvement in yield or growth rate in comparison to control plants, wherein said selected population of progeny plants exhibits said improvement in yield or growth rate and exhibits a nuclear chromosomal DNA methylation pattern that is distinct from a control plant nuclear chromosomal DNA methylation pattern, and wherein the control plant is grown under the same environmental conditions as the selected population of progeny plants and comprises either: (i) progeny of a scion grafted to rootstock that had not been subjected to suppression of MSH1 and DRM2 gene expression; (ii) a whole plant that lacks any root graft and that had not been subjected to suppression of MSH1 and DRM2 gene expression; (iii) a wild-type plant; or (iv) progeny of a plant that is isogenic to the plant source of the scion of the grafted plant, are provided.

Methods for producing a plant exhibiting a useful trait comprising the steps of: (a) obtaining a population of progeny plants from a grafted plant comprising a scion to which a rootstock had been grafted, wherein the rootstock is obtained from a plant or a parent plant thereof had been subjected to suppression of MSH1 and DRM2 gene expression; and, (b) selecting one or more progeny plants from the population, wherein the selected progeny plant exhibit an improvement in the useful trait in comparison to a control plant, thereby producing a plant that exhibits a useful trait, are provided.

### BRIEF DESCRIPTION OF THE DRAWINGS

The accompanying drawings, which are incorporated in and form a part of the specification, illustrate certain embodiments of the present invention. In the drawings:

FIG. 1A, B illustrates the total leaf area of graft progenies (generation 1) at different days after planting. FIG. 1A shows the mean leaf area from Experiment 1 (01/22/2022-Feb. 23, 2022), where 5 independent Col-0/msh1 (open triangle, dashed line), 6 independent Col-0/msh1,drm2 (open square, light dashed line) and 3 independent Col-0/Col-0 (solid circle, solid line) grafts were measured for growth in a reach-in growth chamber divided between 3 shelves. FIG. 1B shows the mean leaf area from Experiment 2 (Apr. 13, 2022-May 5, 2022) where 5 independent Col-0/msh1 (open triangle, dashed line), 6 independent Col-0/msh1,drm2 (open square, light dashed line), and 3 independent Col-0/Col-0 (solid circle, solid line) grafts were screened in a reach-in chamber divided between 3 shelves. In the second experiment, measurements were limited to 4 timepoints due to later gnat infestation. The leaf area of 7-18 plants from each graft was measured using ImageJ software and mean leaf area was calculated as displayed in graph.

FIG. 2 illustrates the total leaf area of graft progenies (generation 2) at different days after planting. The mean leaf area from second generation progenies from 6 independent Col-0/msh1 (open triangle, dashed line), 6 independent Col-0/msh1,drm2 (open square, dashed line) and 3 independent Col-0/Col-0 (solid circle, solid line) grafts (presented in FIG. 1) were measured for growth in a reach-in growth chamber divided between 3 shelves. The leaf area of 7-18 plants from each graft was measured using ImageJ software and mean leaf area was calculated as displayed in the graph.

FIG. 3 illustrates the total leaf area of graft progenies (generation 3) at 35 days after planting. The mean leaf area from third generation progenies from 6 independent Col-0/msh1, 6 independent Col-0/msh1,drm2 and 3 independent Col-0/Col-0 grafts (presented in FIG. 1) were measured for growth in a reach-in growth chamber divided between 3 shelves. The leaf area of 7-18 plants from each graft was measured using ImageJ software and mean leaf area was calculated as displayed in graph.

FIG. 4 illustrates msh1 CRISPR derived mutant Brassica napus plants in R016 genetic background.

FIG. 5 illustrates drm2 CRISPR derived Brassica napus mutant plants in R016 genetic background. Bigger plants in the upper row (2 pots) are wild-type plants, while smaller plants in the lower row (4 pots) are drm2 mutant plants.

### DESCRIPTION

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the phrase “chromosomal modification” refers to any of: a) an “altered chromosomal loci” and an “altered chromosomal locus”; b) “mutated chromosomal loci”, a “mutated chromosomal locus”, “chromosomal mutations” and a “chromosomal mutation”; or c) a transgene.

As used herein, the phrases “altered chromosomal loci” (plural) or “altered chromosomal locus (singular) refer to portions of a chromosome that have undergone a heritable and reversible epigenetic change relative to the corresponding parental chromosomal loci. Heritable and reversible genetic changes in altered chromosomal loci include, but are not limited to, methylation of chromosomal DNA, and in particular, methylation of cytosine residues to 5-methylcytosine residues, and/or post-translational modification of histone proteins, and in particular, histone modifications that include, but are not limited to, acetylation, methylation, ubiquitination, phosphorylation, and sumoylation (covalent attachment of small ubiquitin-like modifier proteins). As used herein, “chromosomal loci” refer to loci in chromosomes located in the nucleus of a cell.

As used herein, the phrase “clonal propagate” refers to a plant or progeny thereof obtained from a plant cell. Clonal propagates can be obtained by methods including but not limited to regenerating whole plants from plant cells, plant embryos, cuttings, and the like. Various techniques used for such clonal propagation include, but are not limited to, meristem culture, somatic embryogenesis, thin cell layer cultures, adventitious shoot culture, and callus culture.

As used herein, the term “comprising” means “including but not limited to.”.

As used herein, the phrase “crop plant” includes, but is not limited to, cereal, seed, grain, fruit, and vegetable crop plants.

As used herein, the phrases “mutated chromosomal loci” (plural), “mutated chromosomal locus” (singular), “chromosomal mutations” and “chromosomal mutation” refer to portions of a chromosome that have undergone a heritable genetic change in a nucleotide sequence relative to the nucleotide sequence in the corresponding parental chromosomal loci. Mutated chromosomal loci comprise mutations that include, but are not limited to, nucleotide sequence inversions, insertions, deletions, substitutions, or combinations thereof. In certain embodiments, the mutated chromosomal loci can comprise mutations that are reversible. In this context, reversible mutations in the chromosome can include, but are not limited to, insertions of transposable elements, defective transposable elements, and certain inversions. In certain embodiments, the chromosomal loci comprise mutations are irreversible. In this context, irreversible mutations in the chromosome can include, but are not limited to, deletions.

As used herein, the term “discrete variation” or “V_{D}” refers to distinct, heritable phenotypic variation, that includes traits of male sterility, dwarfing, variegation, and/or delayed flowering time that can be observed either in any combination or in isolation.

As used herein, the phrase “heterologous sequence”, when used in the context of an operably linked promoter, refers to any sequence or any arrangement of a sequence that is distinct from the sequence or arrangement of the sequence with the promoter as it is found in nature. As such, an MSH1 promoter can be operably linked to a heterologous sequence that includes, but is not limited to, MSH1 sense, MSH1 antisense, combinations of MSH1 antisense and MSH1 sense, and other MSH1 sequences that are distinct from, or arranged differently than, the operably linked sequences of the MSH1 transcription unit as they are found in nature.

As used herein, the term “MSH-dr” refers to leaf variegation, cytoplasmic male sterility (CMS), a reduced growth-rate phenotype, delayed or non-flowering phenotype, increased plant tillering, decreased height, decreased internode elongation, plant tillering, and/or stomatal density changes that are observed in plants subjected to suppression of MSH1 genes. Genes that can be suppressed to produce an MSH-dr phenotype include, but not limited to, MSH1 and both MSH1 and DRM2

As used herein, the term “heterotic group” refers to genetically related germplasm that produce superior hybrids when crossed to genetically distinct germplasm of another heterotic group.

As used herein, the term “progeny” refers to any one of a first, second, third, or subsequent generation obtained from a parent plant or plant cell.

As used herein, the phrase “quantitative variation” or “VQ” refers to phenotypic variation that is observed in individual progeny lines derived from outcrosses of plants where MSH1 expression was suppressed and that exhibit discrete variation to other plants.

As used herein the terms “microRNA” or “miRNA” refers to both a miRNA that is substantially similar to a native miRNA that occurs in a plant as well as to an artificial miRNA. In certain embodiments, a transgene can be used to produce either a miRNA that is substantially similar to a native miRNA that occurs in a plant or an artificial miRNA.

As used herein, the phrase “obtaining a nucleic acid associated with the altered chromosomal locus” refers to any method that provides for the physical separation or enrichment of the nucleic acid associated with the altered chromosomal locus from covalently linked nucleic that has not been altered. In this context, the nucleic acid does not necessarily comprise the alteration (i.e. such as methylation) but at least comprises one or more of the nucleotide base or bases that are altered. Nucleic acids associated with an altered chromosomal locus can thus be obtained by methods including, but not limited to, molecular cloning, PCR, or direct synthesis based on sequence data.

The phrase “operably linked” as used herein refers to the joining of nucleic acid sequences such that one sequence can provide a required function to a linked sequence. In the context of a promoter, “operably linked” means that the promoter is connected to a sequence of interest such that the transcription of that sequence of interest is controlled and regulated by that promoter. When the sequence of interest encodes a protein and when expression of that protein is desired, “operably linked” means that the promoter is linked to the sequence in such a way that the resulting transcript will be efficiently translated. If the linkage of the promoter to the coding sequence is a transcriptional fusion and expression of the encoded protein is desired, the linkage is made so that the first translational initiation codon in the resulting transcript is the initiation codon of the coding sequence. Alternatively, if the linkage of the promoter to the coding sequence is a translational fusion and expression of the encoded protein is desired, the linkage is made so that the first translational initiation codon contained in the 5′ untranslated sequence associated with the promoter is linked such that the resulting translation product is in frame with the translational open reading frame that encodes the protein desired. Nucleic acid sequences that can be operably linked include, but are not limited to, sequences that provide gene expression functions (i.e., gene expression elements such as promoters, 5′ untranslated regions, introns, protein coding regions, 3′ untranslated regions, polyadenylation sites, and/or transcriptional terminators), sequences that provide DNA transfer and/or integration functions (i.e., site specific recombinase recognition sites, integrase recognition sites), sequences that provide for selective functions (i.e., antibiotic resistance markers, biosynthetic genes), sequences that provide scoreable marker functions (i.e., reporter genes), sequences that facilitate in vitro or in vivo manipulations of the sequences (i.e., polylinker sequences, site specific recombination sequences, homologous recombination sequences), and sequences that provide replication functions (i.e., bacterial origins of replication, autonomous replication sequences, centromeric sequences).

As used herein, the phrase “suppressing expression of MSH1 gene(s),” “MSH1 and DRM2 gene expression is suppressed,” and similar refers to any genetic 1 manipulation that provides for decreased levels of functional MSH1 and DRM2 activity in a plant or plant cell relative to the levels of functional MSH1 and DRM2 activity that occur in an otherwise isogenic plant or plant cell that had not been subjected to this genetic or environmental manipulation.

As used herein, the term “transgene”, in the context of a chromosomal modification, refers to any DNA from a heterologous source that has been integrated into a chromosome that is stably maintained in a host cell. In this context, heterologous sources for the DNA include, but are not limited to, DNAs from an organism distinct from the host cell organism, species distinct from the host cell species, varieties of the same species that are either distinct varieties or identical varieties, DNA that has been subjected to any in vitro modification, recombinant DNA, and any combination thereof.

As used herein, the term “non-regenerable” refers to a plant part or plant cell that cannot give rise to a whole plant.

Methods for introducing heritable and epigenetic and/or genetic variation that result in plants that exhibit useful traits are provided herewith along with plants, plant seeds, plant parts, plant cells, and processed plant products obtainable by these methods. In certain embodiments, methods provided herewith can be used to introduce epigenetic and/or genetic variation into varietal or non-hybrid plants that result in useful traits as well as useful plants, plant parts including, but not limited to, seeds, plant cells, and processed plant products that exhibit, carry, or otherwise reflect benefits conferred by the useful traits. In other embodiments, methods provided herewith can be used to introduce epigenetic and/or genetic variation into plants that are also amenable to hybridization.

In certain embodiments, the methods for introducing heritable epigenetic or genetic variation in a plant or progeny thereof can comprise the step of grafting rootstock obtained from a plant or a parent plant thereof wherein MSH1 and DRM2 gene expression is suppressed to a scion. In certain embodiments of any of the aforementioned methods, the heritable epigenetic variation provides a useful trait is selected from the group consisting of improved yield, delayed flowering, non-flowering, increased biotic stress resistance, increased abiotic stress resistance, enhanced lodging resistance, enhanced growth rate, enhanced biomass, enhanced tillering, enhanced branching, delayed flowering time, delayed senescence, increased flower number, improved architecture for high density planting, improved photosynthesis, increased root mass, increased cell number, improved seedling vigor, improved seedling size, increased rate of cell division, improved metabolic efficiency, and increased meristem size in comparison to the control plant. In certain embodiments, the plant, progeny of the plant, or scion contain(s) one or more epigenetic changes in one or more nuclear chromosomes, wherein the epigenetic changes are absent from nuclear chromosomes of the control plant or are absent from nuclear chromosomes of a plant from which the scion was obtained. In certain embodiments, the epigenetic change(s) are also present in the rootstock that had been subjected to perturbation of plastid function. In certain embodiments, the epigenetic changes in the plant, progeny of the plant, scion, or rootstock are associated with the improvement in the useful trait. In certain embodiments, the epigenetic changes in the plant, progeny of the plant, scion, or rootstock induced by suppression of the MSH1 and DRM2 genes are associated with the improvement in the useful trait. In certain embodiments, the plant, progeny of the plant, scion, or rootstock contain(s) one or more epigenetic changes in one or more nuclear chromosomes that are absent from nuclear chromosomes of rootstock obtained from a plant or are absent from nuclear chromosomes of a parent plant thereof had not been subjected to perturbation of plastid function. In certain embodiments, the plant, progeny of the plant, scion and/or the rootstock exhibit CG hypermethylation of a region encompassing a MSH1 locus in comparison to a control plant that had not been subjected to the MSH1 and DRM2 gene suppression. In certain embodiments, the plant, progeny of the plant, scion and/or the rootstock exhibit pericentromeric CHG hyper-methylation in comparison to a control plant that had not been subjected to the MSH1 and DRM2 gene suppression. In certain embodiments, the plant, progeny of the plant, scion and/or the rootstock exhibit CG hypermethylation and/or CHG hypermethylation at one or more nuclear chromosomal loci in comparison to corresponding nuclear chromosomal loci of a control plant that had not been subjected to the MSH1 and DRM2 gene suppression. In certain embodiments, the plant is selected from the group consisting of a crop plant, a tree, a bush, and a vine. In certain embodiments, the crop plant is selected from the group consisting of corn, soybean, cotton, canola, wheat, rice, tomato, tobacco, millet, potato, sugarbeet, cassava, alfalfa, barley, oats, sugarcane, sunflower, strawberry, and sorghum. In certain embodiments, the tree is selected from the group consisting of an apple, apricot, grapefruit, orange, peach, pear, plum, lemon, coconut, poplar, eucalyptus, date palm, palm oil, pine, and an olive tree. In certain embodiments, the bush is selected from the group consisting of a blueberry, raspberry, and blackberry bush. In certain embodiments, the vine is a grape vine. Also provided are plants or progeny thereof obtained by any of the aforementioned methods. Also provided are plant parts obtained from the plant or progeny thereof that were made by any of the aforementioned methods.

Without seeking to be limited by theory, DRM2 in Arabidopsis and other plants encodes for a methyltransferase that functions in RdDM-targeted DNA methylation. Inactivation of DRM2 may partially inactivate the RdDM pathway within the rootstock and prevent incorporation of msh1-induced small RNAs (sRNAs) to rootstock epigenomic processes, thereby permitting more efficient sRNA transfer to the scion. Since the grafting is conducted during flowering, source-sink transition in the plant likely also facilitates sRNA transmission from rootstock to scion.

Also provided herein are grafted plants comprising a scion to which a rootstock had been grafted, wherein the rootstock is obtained from a plant or a parent plant thereof wherein MSH1 and DRM2 gene expression is suppressed, as well as progeny plants and clonal propagates obtained from the grafted plant. Such rootstocks can be also used to introduce epigenetic and/or genetic variation into varietal or non-hybrid plants that result in useful traits as well as useful plants, plant parts including, but not limited to, seeds, plant cells, and processed plant products that exhibit, carry, or otherwise reflect benefits conferred by the useful traits. In other embodiments, such rootstocks can also be used to introduce epigenetic and/or genetic variation into plants that are also amenable to hybridization.

Rootstocks useful for introducing epigenetic and/or genetic variation into plants can be obtained from a variety of rootstock source plants wherein MSH1 and DRM2 gene expression is suppressed. In certain embodiments, the rootstock source plant is a plant that had itself been subjected to suppression of MSH1 and DRM2 gene expression. In other embodiments, the rootstock source plant is the progeny of a parental plant that had itself been subjected to suppression of MSH1 and DRM2 gene expression. Various methods of making rootstock source plants by suppression of MSH1 and DRM2 gene expression are provided herein. Plants that can serve as rootstock source plants and methods of making such plants are also disclosed in US Patent Application Publication No. 20120284814, which is specifically incorporated herein by reference in its entirety, and elsewhere in this disclosure. The use of plants with useful traits and methods of making such plants disclosed in para. [0072], [0085], and in US Patent Application Publication No. 20120284814 as rootstock sources is specifically provided, and each of those paragraphs is specifically incorporated herein by reference in their entireties.

In certain embodiments where the rootstock source plant, or a parental plant thereof, had been subjected to suppression of MSH1 and DRM2 gene expression, a population of progeny plants obtained from the grafted plant are screened and individual progeny plants are selected for one or more useful traits. Such populations of progeny plants can be obtained by methods including, but not limited to, selfing or outcrossing the grafted plant comprising the rootstock to obtain seed that give rise to the population. Such populations of progeny plants can also be obtained by methods including, but not limited to, growing a population of plants that are derived from independent clonal propagates obtained from the grafted plant comprising the rootstock. Such selected individual progeny plants that exhibit the useful trait can then be sexually or asexually propagated to yield populations of plants that exhibit the useful trait or seed lots that exhibit or harbor the useful trait. Such sexual propagation can be accomplished by selfing or outcrossing the selected individual progeny plants that exhibit the useful trait.

In certain embodiments where the rootstock source plant is the progeny of a parental plant that had been subjected to suppression of MSH1 and DRM2 gene expression, the rootstock source plant itself can be a plant that was selected for one or more useful traits. Grafting rootstock from a plant that had been selected for a useful trait to a scion that does not exhibit the trait can impart the trait to the resultant grafted plant or to progeny thereof. Resultant grafted plants or progeny thereof that exhibit the useful trait can then be sexually or asexually propagated to yield populations of plants that exhibit the useful trait or seed lots that exhibit or harbor the useful trait.

In grafted plants or progeny thereof, suppression of MSH1 and DRM2 gene expression in the rootstock can be continuous and ongoing or can be transient. Non-limiting and exemplary methods for effecting continuous and ongoing suppression of MSH1 and DRM2 gene expression in the rootstock include suppressing expression of MSH1 and DRM2 genes with loss-of-function mutations in the endogenous gene and/or with a transgene that yields a product that suppresses expression of the endogenous gene. Alternatively, the suppression of MSH1 and DRM2 gene expression in the rootstock can be transient or have occurred in a parental plant from which the rootstock was obtained but not in the rootstock that was used in the graft. Non-limiting and exemplary methods for effecting transiently suppressing MSH1 and DRM2 gene function in the rootstock include suppressing expression of and endogenous MSH1 and a DRM2 gene with a transgene that provides for inducible or repressible expression of a product that suppresses expression of the endogenous MSH1 and a DRM2 gene, with a transgene that can be excised, or with a heterozygous transgene insert that is removed from the rootstock by segregation. Any of the methods described herein for restoring plastid function after perturbation can be used to generate rootstock used in certain embodiments.

Grafting can be effected by any method that provides for establishment of a vascular connection between the rootstock and the scion. Methods of grafting that can be used to effect the connection between the scion and the rootstock include, but are not limited to, apical graftage, side graftage, bark graftage, and root graftage. Such methods for effecting grafts of scions to rootstock are disclosed in “Plant Propagation: Principles and Practices; Chapter 12: Techniques of Grafting” Ed. Hartman, Kester, Davies, and Geneve, 7th Edition. Methods for effecting grafts of monocot plant scions to rootstocks that can be used with the scions and rootstocks provided herein are disclosed in Muzik and La Rue, The Grafting of Large Monocotyledonous Plants, Science 116, No. 3022:589-591, 1952.

Rootstocks subjected to MSH1 and DRM2 gene suppression or obtained from a parental plant that had been subjected to MSH1 and DRM2 gene suppression can exhibit modifications of one or more nuclear chromosomes. In certain embodiments, such rootstocks can exhibit characteristic DNA methylation and/or gene transcription patterns that occur in plants subjected to suppression of an MSH1 target gene. Such characteristic DNA methylation and/or gene transcription patterns that occur in plants or seeds subjected to suppression of an MSH1 target gene can include, but are not limited to, those patterns disclosed in U.S. Pat. No. 10,767,188, which is incorporated herein by example in its entirety. In certain embodiments, rootstock of first generation progeny of a plant subjected to suppression of an MSH1 gene will exhibit CG differentially methylated regions (DMR) of various discrete chromosomal regions that include, but are not limited to, regions that encompass the MSH1 locus. In certain embodiments, a CG hypermethylated region that encompasses the MSH1 locus will be about 5 to about 8 MBp (mega base pairs) in length. In certain embodiments, rootstock of first generation progeny of a plant subjected to suppression of a MSH1 and DRM2 gene will also exhibit changes in plant defense and stress response gene expression. In certain embodiments, a rootstock, a scion grafted thereto, and/or a plant cell, a seed, a progeny plant, plant populations, seed populations, and/or processed products obtained therefrom that has been subject to suppression of a MSH1 and DRM2 gene will exhibit methylation repatterning similar to methylation repatterning observed when MSH1 is suppressed (Kundariya et al. 2020 and Kundariya et al. 2022). Such methylation repatterning can be assessed by comparing the methylation status of a sample from rootstocks, scions of plants grafted to root stocks, plants or seed that had been subjected to suppression of MSH1 and DRM2 genes, or a sample from progeny plants or seed derived therefrom, to a sample from control plants or seed that had not been subjected to suppression of MSH1 and DRM2 genes. In this and certain other contexts, such control plants include, but are not limited to, plants, grafted plants, scions thereof and rootstocks thereof that had not been subjected to MSH1 and DRM2 gene suppression. In certain embodiments, such aforementioned changes in the methylation patterns exhibited by scions that are grafted to the rootstocks, or exhibited by a plant cell, a seed, a progeny plant, plant populations, seed populations, and/or processed products obtained from the grafted plant, be used to monitor the effectiveness of the graft in transmitting desirable epigenetic changes or to identify a plant cell, a seed, a progeny plant, plant populations, seed populations, and/or processed products obtained from the grafted plant.

Also provided herein are various methods for producing a plant exhibiting a useful trait that comprise crossing grafted plants comprising a scion grafted to rootstock that had been subjected to perturbation of plastid function with another plant, or crossing progeny plants obtained from the grafted plant with another plant, and selecting one or more progeny plants obtained from the cross for an improvement in the useful trait in comparison to a control plant. In certain embodiments, the second plant can also be a grafted plant comprising a scion grafted to rootstock that had been subjected to perturbation of plastid function, a progeny plants obtained from a grafted plant comprising a scion grafted to rootstock that had been subjected to perturbation of plastid function, any other ungrafted plant that had been subjected to perturbation of plastid function, or any other ungrafted plant obtained from one or more parental plants that had been subjected to perturbation of plastid function. Such second plants can be plants that were selected for a useful trait and that were progeny of any plant or grafted plant that had subjected to perturbation of plastid function. Control plants used as comparators to identify progeny of the cross that exhibit an improvement in the useful trait include, but are not limited to: progeny of a cross between a plant which lacks a graft to the rootstock and a plant that is isogenic to the second plant, progeny of a self of a plant that lacks a graft to the rootstock, progeny of a self of the second plant; progeny of a cross between a plant that is isogenic to the plant source of the scion of the grafted plant and a plant that is isogenic to the second plant; and, progeny of a cross between a plant that is isogenic to the plant source of the scion of the grafted plant and that is isogenic to the plant source of a scion of the second plant when the second plant is a grafted plant. Also provided are methods where at least the scion of the first plant is from a different heterotic group than the second plant or where at least the scion of the first plant is from the same heterotic group as the second plant.

Also provided herein are various methods for producing a plant exhibiting a useful trait that comprise selfing grafted plants comprising a scion grafted to rootstock that had been subjected to perturbation of plastid function with another plant, or selfing progeny plants obtained from the grafted plant, and selecting one or more progeny plants obtained from the self for an improvement in the useful trait in comparison to a control plant to produce a plant exhibiting a useful trait. In certain embodiments, the selfed plant is a grafted plant where the rootstock source plant is the progeny of a parental plant that had been subjected to suppression of MSH1 and DRM2 gene expression and the rootstock source plant itself was selected for and exhibits one or more useful traits. Control plants used as comparators to identify progeny of the self that exhibit an improvement in the useful trait include, but are not limited to: progeny of a self of a plant which lacks a graft to the rootstock, progeny of a self of a plant that has a graft to rootstock that had not been subjected to suppression of MSH1 and DRM2 gene expression, and progeny of a self of a plant that is isogenic to the plant source of the scion of the grafted plant.

In certain embodiments, useful traits provided herein can be exhibited to a greater extent in subsequent generations of plants that are obtained from any of the grafted plants, parental plants, or parental plant cells that had been subjected to suppression of MSH1 and DRM2 gene expression that are provided herein. As such, a given initial plant obtained from a parent plant that was subjected to suppression of MSH1 and DRM2 gene expression can be selfed to obtain first, second, third, or later generations of progeny that exhibit a given useful trait to a greater extent in comparison to either the initial plant or in comparison to a control plant. An initial grafted plant comprising a scion grafted to rootstock subjected to suppression of MSH1 and DRM2 gene expression or to rootstock obtained from a parent plant that had been subjected to suppression of MSH1 and DRM2 gene expression can be selfed to obtain first, second, third, or later generations of progeny that exhibit a given useful trait to a greater extent in comparison to either the grafted initial plant or in comparison to a control plant. In other embodiments, a given initial plant obtained from a parent plant that was subjected to suppression of MSH1 and DRM2 gene expression can be outcrossed to obtain F1, F2, F3, or later generations of progeny that exhibit a given useful trait to a greater extent in comparison to either the initial plant or in comparison to a control plant. In certain embodiments, a useful trait harbored by an initial plant or an initial grafted plant is not exhibited, or is exhibited to a lesser degree extent, in the initial plant or an initial grafted plant. However, the useful trait harbored by such an initial plant or an initial grafted plant is exhibited or is exhibited to a greater extent in progeny obtained by outcrossing the initial plant or the initial grafted plant to another plant. A useful trait harbored by such an initial plant or an initial grafted plant can also be exhibited or is exhibited to a greater extent in progeny obtained by selfing the initial plant or the initial grafted plant. In certain embodiments, plants or grafted plants that are selfed or outcrossed can be inbred lines. In certain embodiments, a useful trait harbored by an inbred line is not exhibited, or is exhibited to a lesser degree extent, in the inbred line. However, the useful trait harbored by such inbred lines is exhibited or is exhibited to a greater extent in progeny obtained by outcrossing the inbred line to another plant. An initial grafted plant comprising a scion grafted to rootstock subjected to suppression of MSH1 and DRM2 gene expression or to rootstock obtained from a parent plant that had been subjected to suppression of MSH1 and DRM2 gene expression can be outcrossed to obtain F1, F2, F3, or later generations of progeny that exhibit a given useful trait to a greater extent in comparison to either the initial grafted plant or in comparison to a control plant. Outcrosses of such initial plants or grafted plants can be to isogenic plants or to genetically distinct plants. In the methods provided herein, initial or subsequent generations of progeny obtained from such selfs or crosses can thus be selected for useful traits. The methods provided herein also permit the identification of plants that harbor, but do not necessarily exhibit to a full extent, various useful traits.

Clonal propagates can be obtained by methods including, but not limited to, regenerating whole plants from plant cells, plant embryos, cuttings, and the like that are obtained from scions of the grafted plants provided herein or progeny thereof. Various techniques used for such clonal propagation include, but are not limited to, meristem culture, somatic embryogenesis, thin cell layer cultures, adventitious shoot culture, and callus culture. In certain embodiments, clonal propagation is effected by placing sterile plant cells, plant embryos, cuttings, and the like in sterile plant culture media containing suitable salts, sugars, and plant growth regulators to support regeneration of a plant or plant part. Such techniques suitable for clonal propagation are often referred to as “micropropagation.” Typically, cytokinins are used to stimulate shoot formation while auxins are used to stimulate root formation in the cultured material. Techniques that can be used for clonal propagation of potato plants provided herein include, but are not limited to, methods where sterile cuttings from tubers are multiplied in a modified Murashige-Skoog media to produce micropropagated plants that can be explanted to soil to produce micro-tubers that can then serve as seed potato tubers (Ahloowalia, Euphytica 75:163, 1994). Other methods that can be used for clonal propagation of potato plants provided herein include, but are not limited to, methods where nodal, meristem, or shoot tip tissues are cultured and multiplied (Rosell, G. et al. Potato Research 30:111, 1987, and references cited therein). Still other methods that can be used for clonal propagation of potato plants provided herein include, but are not limited to, methods where nodal segments are cultured in a bioreactor to mass produce microtubers that can then serve as seed potato tubers (Piao et al., Current Science 84 (8): 1129, 2003). Techniques that can be used for clonal propagation of sugar beet plants provided herein include, but are not limited, to petiole explant propagation (Grieve, et al. Plant Growth Regulation 21:15, 1997), or propagation of leaf blades, apical meristems, stalk, embryo, or hypocotyls (Mezei, S. et al. Biotechnology & Biotechnological Equipment, 20:1, 9-14, 2006).

In certain embodiments, methods provided herewith involve suppressing expression of MSH1 and DRM2 target genes, restoring expression of a functional MSH1 and DRM2 gene, and selecting progeny plants that exhibit one or more useful traits. In certain embodiments, these useful traits are associated with either one or more altered chromosomal loci that have undergone a heritable and reversible epigenetic changes.

In certain embodiments, methods for selectively suppressing expression of MSH1 and DRM2 target genes in sub-populations of cells found in plants that contain plastids referred to herein as “sensory plastids” are provided. Sensory plastids are plastids that occur in cells that exhibit preferential expression of at least the MSH1 promoter. In certain embodiments, MSH1 and other promoters active in sensory plastids can thus be operably linked to a heterologous sequence that perturbs plastid function to effect selective suppression of genes in cells containing the sensory plastids. In addition to the distinguishing characteristic of expressing MSH1, such cells containing sensory plastids can also be readily identified as their plastids are only about 30-40% of the size of the chloroplasts contained within mesophyll cells. Other promoters believed to be active in sensory plastids include, but are not limited to, PPD3 gene promoters. Selective suppression of MSH1 and DRM2 genes in cells containing sensory plastids can trigger epigenetic changes that provide useful plant traits.

Exemplary MSH1 and DRM2 target genes from Arabidopsis with the accession number for the corresponding sequences in the Arabidopsis genome database (on the world wide web at the address “Arabidopsis.org”) and orthologous MSH1 and DRM2 genes which can be targeted for suppression in other crop plants are provided in Table 1. Orthologous genes from many crop species can be obtained through the BLAST comparison of the protein sequences of the Arabidopsis genes above to the genomic databases (NCBI and publicly available genomic databases for specific crop species), as well as from the specific names of the subunits. Specifically the genome, cDNA, or EST sequences are available for apples, beans, barley, Brassica napus, rice, Cassava, Coffee, Eggplant, Orange, sorghum, tomato, cotton, grape, lettuce, tobacco, papaya, pine, rye, soybean, sunflower, peach, poplar, scarlet bean, spruce, cocoa, cowpea, maize, onion, pepper, potato, radish, sugarcane, wheat, and other species at the following internet or world wide web addresses:
“compbio.dfci.harvard.edu/tgi/plant.html”; “genomevolution.org/wiki/index.php/Sequenced_plant_genomes”; “ncbi.nlm.nih.gov/genomes/PLANTS/PlantList.html”; “plantgdb.org/”; “arabidopsis.org/portals/genAnnotation/other_genomesr”; “gramene.org/resources/”; “genomenewsnetwork.org/resources/sequenced_genomes/genome_guide_p1.shtml”; “jgi.doe.gov/programs/plants/index.jsr”; “chibba.agtec.uga.edu/duplication/”; “mips.helmholtz-muenchen.de/plant/genomes.jsp”; “science.co.il/biomedical/Plant-Genome-Databases.asp”; “jcvi.org/cms/index.php?id=16”; “rapdb.dna.affrc.go.jp/”; “ensembl.sorghumbase.org”; “genoscope.cns.fr”; “solgenomics.net”; “maizegdb.org”; and “phyto5.phytozome.net/Phytozome_resources.php”.

TABLE 1
MSH1 and DRM2 genes from Arabidopsis and other plants.

| MSH1 | | |
|---|---|---|
| Genes | | |
| SEQ | | |
| ID NO: | Plant | Database Accession |
| 1 | Arabidopsis thaliana | AT3G24320 (MSH1) |
| 2 | Glycine max (Williams 82) | Glyma.10g130700.1 |
| | | (MSH1) |
| 3 | Glycine max (Williams 82) | Glyma.20g073400 |
| | | (MSH1 truncated) |
| 4 | Brassica napus (darmor-bzh) | BnaC07p12050 |
| 5 | Brassica napus (darmor-bzh) | BnaA07p07990 |
| 6 | Solanum lycopersicum (tomato) | Solyc09g090870 |
| | | (GenBank: AY866434) |
| 7 | Oryza sativa (rice) | Os04t0507000 |
| 8 | Sorghum bicolor (BTx623) | Sobic.006G140400 |
| 9 | Zea mays | GRMZM2G360873 |
| DRM2 | | |
| Genes | | |
| SEQ | | |
| ID NO: | Plant | NCBI Accession |
| 10 | Arabidopsis thaliana | AT5G14620 (DRM2) |
| 11 | Glycine max (Williams 82) | Glyma.05g005600 (DRM2) |
| 12 | Glycine max (Williams 82) | Glyma.19g006100 (DRM2 |
| | | homolog) |
| 13 | Brassica napus (darmor-bzh) | BnaC09p62500 |
| 14 | Brassica napus (darmor-bzh) | BnaA10p24030 |
| 15 | Solanum lycopersicum (tomato) | Solyc10g078190 |
| | | (SlDRM1L1) |
| 16 | Solanum lycopersicum (tomato) | Solyc04g005250 (SlDRM1L) |
| 17 | Solanum lycopersicum (tomato) | Solyc02g062740 (SlDRM5) |
| 18 | Oryza sativa (rice) | Os03g0110800 |
| 19 | Sorghum bicolor (BTx623) | Sobic.003G124000 |
| 20 | Zea mays | GRMZM2G092497 |
| | | (ZmDRM1; Zmet3) |
| 21 | Zea mays | GRMZM2G137366 |
| | | (ZmDRM2; Zmet7) |

In general, methods provided herewith for introducing epigenetic and/or genetic variation in plants simply require that MSH1 and DRM2 target gene expression be suppressed for a time sufficient to introduce the variation and/or in appropriate subsets of cells (e.g., cells containing sensory plastids). As such, a wide variety of MSH1 and DRM2 gene suppression methods can be employed to practice the methods provided herewith and the methods are not limited to a particular suppression technique.

Sequences of MSH1 and DRM2 genes or fragments thereof from Arabidopsis and various crop plants are provided herewith (e.g., in Table 1). In certain embodiments, such genes may be used directly in either the homologous or a heterologous plant species to provide for suppression of the endogenous MSH1 and DRM2 target gene in either the homologous or heterologous plant species. A non-limiting, exemplary demonstration where an exemplary MSH1 gene from one species was shown to be effective in suppressing the endogenous MSH1 gene in both a homologous and a heterologous species is provided by Sandhu et al. 2007, where a transgene that provides for an MSH1 inhibitory RNA (RNAi) with tomato MSH1 sequences was shown to inhibit the endogenous MSH1 genes of both tomato and tobacco. A transgene that provides for a MSH1 and/or DRM2 gene inhibitory RNA (RNAi) with maize MSH1 and/or DRM2 gene sequences can be used in certain embodiments to inhibit the endogenous MSH1 and DRM2 gene genes of millet, sorghum, and maize/or gene genes from other plants including, but not limited to, cotton, canola, wheat, barley, flax, oat, rye, turf grass, sugarcane, alfalfa, banana, broccoli, cabbage, carrot, cassava, cauliflower, celery, citrus, a cucurbit, eucalyptus, garlic, grape, onion, lettuce, pea, peanut, pepper, potato, poplar, pine, sunflower, safflower, soybean, blackberry, blueberry, sugar beet, sweet potato, tobacco, strawberry, sugar beet, sweet potato, Jatropha, Camelina, and Agave can be obtained by a variety of techniques and used to suppress expression of either the corresponding MSH1 and DRM2 gene in those plants or the MSH1 and DRM2 gene in a distinct plant. Methods for obtaining MSH1 and DRM2 genes for various plants include, but are not limited to, techniques such as: i) searching amino acid and/or nucleotide sequence databases comprising sequences from the plant species to identify the MSH1 and DRM2 gene by sequence identity comparisons; ii) cloning the MSH1 and DRM2 gene by either PCR from genomic sequences or RT-PCR from expressed RNA; iii) cloning the MSH1 and DRM2 gene from a genomic or cDNA library using PCR and/or hybridization based techniques; iv) cloning the MSH1 and DRM2 gene from an expression library where an antibody directed to the MSH1 and DRM2 gene protein is used to identify the MSH1 and DRM2 gene containing clone; v) cloning the MSH1 and DRM2 gene by complementation of an MSH1 and DRM2 gene mutant or MSH1 and DRM2 gene deficient plant; or vi) any combination of (i), (ii), (iii), (iv), and/or (v). The DNA sequences of the target genes can be obtained from the promoter regions or transcribed regions of the target genes by PCR isolation from genomic DNA, or PCR of the cDNA for the transcribed regions, or by commercial synthesis of the DNA sequence. RNA sequences can be chemically synthesized or, more preferably, by transcription of suitable DNA templates. Recovery of the MSH1 and DRM2 gene from the plant can be readily determined or confirmed by constructing a plant transformation vector that provides for suppression of the gene, transforming the plants with the vector, and determining if plants transformed with the vector exhibit the characteristic responses that are typically observed in various plant species when MSH1 expression is suppressed that include leaf variegation, cytoplasmic male sterility (CMS), a reduced growth-rate phenotype, and/or delayed or non-flowering phenotype. The characteristic responses of MSH1 suppression have been described previously as developmental reprogramming or “MSH-dr1” (Xu et al. Plant Physiol. Vol. 159:711-720, 2012).

In certain embodiments, MSH1 and DRM2 genes or fragments thereof used in the methods provided herein will have nucleotide sequences with at least 50%, 60%, 70%, 80%, 90%, 95%, 98%, 99%, or 100% nucleotide sequence identity to one or more of the MSH1 and DRM2 genes or fragments thereof provided herein that include, but are not limited to, genes provided in Table 1 and orthologs thereof found in various crop plants. In certain embodiments, the MSH1 and DRM2 genes or fragments thereof used in the methods provided herein encode MSH1 and DRM2 gene proteins or portions thereof will have amino acid sequences with at least 50%, 60%, 70%, 80%, 90%, 95%, 98%, 99%, or 100% amino acid sequence identity to one or more of the MSH1 and DRM2 gene proteins provided herein that include, but are not limited to, the MSH1 and DRM2 gene proteins encoded by genes provided in Table 1.

It is anticipated that MSH1 and DRM2 gene nucleic acid fragments of 18 to 20 nucleotides, but more preferably 21 nucleotides or more, can be used to effect suppression of the endogenous MSH1 and DRM2 gene. In certain embodiments, MSH1 and DRM2 gene nucleic acid fragments of at least 18, 19, 20, or 21 nucleotides to about 50, 100, 200, 500, or more nucleotides can be used to effect suppression of the endogenous MSH1 and DRM2 gene. Regions of 20, 50, 100, 500, or more by are suitable for this purpose, with lengths of 100 to 300 bases of the target gene sequences preferable, and lengths of 300 to 500 bp or more being most preferable. For use in a hairpin or inverted repeat knockdown design, a spacer region with a sequence not related to the sequence of the genome of the target plant can be used. A hairpin construct containing 300 to 500 bp or more of a target gene sequence in the antisense orientation, followed by a spacer region whose sequence is not critical but can be a intron or non-intron. If the spacer is an intron, the caster bean catalase intron which is effectively spliced in both monocots and dicots (Tanaka, Mita et al. Nucleic Acids Res 18 (23): 6767-6770, 1990), is known to those skilled in the art and is useful for the present embodiment. After the spacer the same target gene sequence in the sense orientation is present, such that the antisense and sense strands can form a double stranded RNA after transcription of the transcribed region. The target gene sequences are followed by a polyadenylation region. 3′ polyadenylation regions known to those skilled in the art to function in monocots and dicot plants include but are not limited to the Nopaline Synthase (NOS) 3′ region, the Octopine Synthase (OCS) 3′ region, the Cauliflower Mosaic Virus 35S 3′ region, the Mannopine Synthase (MAS) 3′ region. Additional 3′ polyadenylation regions from monocotyledonous genes such as those from rice, sorghum, wheat, and maize are available to those skilled in the art to provide similar polyadenylation region and function in DNA constructs in the present embodiments. In certain embodiments, a transgene designed to suppress a target gene in dicots is designed to have the following order: promoter/antisense to target gene/catalase intron/sense gene A/polyadenylation region. In embodiments where a gene is designed to suppress a target gene in monocots can have the following order: promoter/intron for monocots/antisense to target gene/catalase intron/sense gene A/polyadenylation region.

Sequences that provide for suppression of a MSH1 and DRM2 gene can include sequences that exhibit complementarity to either strand of the promoter, 5′ or 3′ untranslated region, intron, coding regions, and/or any combination thereof. A target gene promoter region for gene suppression can include the transcription start site, the TATA box, and upstream regions. The promoter region for gene silencing can be about 20, 50, 80, or 100 nucleotides in length, and more preferably is about 100 to 500 nucleotides in length. The promoter region used for such suppression can be from different regions in the upstream promoter, preferably containing at least about 500 nucleotides upstream from the start of transcription, and most preferably containing at least about 500 nucleotides upstream from the start of translation of the native coding region of the native gene. This would include the UTR which may or may not be part of the promoter. A description of various recombinant DNA constructs that target promoter and/or adjoining regions of target genes are described in U.S. Pat. No. 8,293,975, which is incorporated herein by reference in its entirety.

In certain embodiments, suppression of MSH1 and DRM2 gene in a plant is effected with a transgene. Transgenes that can be used to suppress expression of MSH1 and DRM2 gene include, but are not limited to, transgenes that produce dominant-negative mutants of a MSH1 and DRM2 gene, a small inhibitory RNA (siRNA), a microRNA (miRNA), a co-suppressing sense RNA, and/or an anti-sense RNA that provide for inhibition of the endogenous MSH1 and DRM2 gene. U.S. patents incorporated herein by reference in their entireties that describe suppression of endogenous plant genes by transgenes include U.S. Pat. Nos. 7,109,393, 5,231,020 and 5,283,184 (co-suppression methods); and U.S. Pat. Nos. 5,107,065 and 5,759,829 (antisense methods). In certain embodiments, transgenes specifically designed to produce double-stranded RNA (dsRNA) molecules with homology to the MSH1 and DRM2 gene can be used to decrease expression of the endogenous MSH1 and DRM2 gene. In such embodiments, the sense strand sequences of the dsRNA can be separated from the antisense sequences by a spacer sequence, preferably one that promotes the formation of a dsRNA (double-stranded RNA) molecule. Examples of such spacer sequences include, but are not limited to, those set forth in Wesley et al., Plant J., 27 (6): 581-90 (2001), and Hamilton et al., Plant J., 15:737-746 (1998). One exemplary and non-limiting vector that has been shown to provide for suppression of an MSH1 target gene in tobacco and tomato has been described by Sandhu et al., 2007 where an intron sequence separates the sense and antisense strands of the MSH1 target gene sequence. The design of recombinant DNA constructs for suppression of gene expression are also described in Helliwell, C. and P. Waterhouse (2003). “Constructs and methods for high-throughput gene silencing in plants.” Methods 30 (4): 289-295.

In certain embodiments, transgenes that provide for MSH1 and DRM2 gene suppression can comprise regulated promoters that provide for either induction or downregulation of operably linked MSH1 and DRM2 gene inhibitory sequences. In this context, MSH1 and DRM2 gene inhibitory sequences can include, but are not limited to, dominant-negative mutants of MSH1 and DRM2 gene, a small inhibitory RNA (siRNA), a microRNA (miRNA), a co-suppressing sense RNA, and/or an anti-sense RNA that provide for inhibition of the endogenous MSH1 and DRM2 gene of a plant. Such promoters can provide for suppression of MSH1 and DRM2 gene during controlled time periods by either providing or withholding the inducer or down regulator. Inducible promoters include, but are not limited to, a PR-1a promoter (U.S. Patent Application Publication Number 20020062502) or a GST II promoter (WO 1990/008826 A1). In other embodiments, both a transcription factor that can be induced or repressed as well as a promoter recognized by that transcription factor and operably linked to the MSH1 and DRM2 gene inhibitory sequences are provided. Such transcription factor/promoter systems include, but are not limited to: i) RF2a acidic domain-ecdysone receptor transcription factors/cognate promoters that can be induced by methoxyfenozide, tebufenozide, and other compounds (U.S. Patent Application Publication Number 20070298499); ii) chimeric tetracycline repressor transcription factors/cognate chimeric promoters that can be repressed or de-repressed with tetracycline (Gatz, C., et al. (1992). Plant J. 2, 397-404), and the like.

In certain embodiments, a promoter that provides for selective expression of a heterologous sequence that suppresses expression of the target gene in cells containing sensory plastids is used. In certain embodiments, this promoter is an Msh1 or a PPD3 promoter. In certain embodiments, this promoter is an Msh1 or a PPD3 promoter and the operably linked heterologous sequence suppresses expression of a target gene provided in U.S. Pat. No. 10,767,188, incorporated herein by reference in its entirety. Msh1 promoters that can be used to express heterologous sequences in cells containing sensor plastids include, but are not limited to, the Arabidopsis, sorghum, tomato, and maize promoters provided herewith or provided in U.S. Pat. No. 10,767,188, incorporated herein by reference in its entirety, as well as functional derivatives thereof that likewise provide for expression in cells that contain sensor plastids. In certain embodiments, deletion derivatives of the Msh1 promoters comprising about 1500 Bp, 1000 Bp, or about 750 Bp can also be used to express heterologous sequences. PPD3 promoters that can be used to express heterologous sequences in cells containing sensor plastids include, but are not limited to, the Arabidopsis, rice, and tomato promoters provided in provided in U.S. Pat. No. 10,767,188, incorporated herein by reference in its entirety as well as functional derivatives thereof that provide for expression in cells that contain sensor plastids. In certain embodiments, deletion derivatives of the Msh1 promoters comprising about 800 Bp, 600 Bp, or about 500 Bp of the PPD3 promoters can also be used to express heterologous sequences. In certain embodiments, the aforementioned PPD3 promoters and an additional 200, 500, or 1000 base pairs of the endogenous 5′PPD3 promoter sequences can be used to express heterologous sequences. Additional 200, 500, or 1000 base pairs of the endogenous 5′PPD3 promoter sequences can be obtained by methods including, but not limited to, retrieval of sequences from databases provided herein and recovery of the adjoining promoter DNA by PCR amplification of genomic template sequences or by direct synthesis. In certain embodiments, recombinant DNA constructs for suppression of dicot target genes can comprise a MSH1 or PPD3 promoter from a dicotyledonous species such as Arabidopsis, soybeans or canola, is attached to a hairpin construct containing 300 to 500 bp or more of a target gene sequence in the antisense orientation, followed by a spacer region whose sequence is not critical but can be an intron or non-intron. The caster bean catalase intron (Tanaka, Mita et al. Nucleic Acids Res 18 (23): 6767-6770, 1990), can be used as a spacer in certain embodiments. After the spacer the same target gene sequence in the sense orientation is present, such that the antisense and sense strands can form a double stranded RNA after transcription of the transcribed region. The target gene sequences are followed by a polyadenylation region. Various 3′ polyadenylation regions known to function in monocots and dicot plants include but are not limited to the Nopaline Synthase (NOS) 3′ region, the Octopine Synthase (OCS) 3′ region, the Cauliflower Mosaic Virus 35S 3′ region, the Mannopine Synthase (MAS) 3′ region. In certain embodiments recombinant DNA constructs for suppression of monocot target genes can comprise MSH1 or PPD3 promoter from a monocot species such as rice, maize, sorghum or wheat can either be attached directly to the hairpin region or to a monocot intron before the hairpin region. Monocot introns that are beneficial to gene expression when located between the promoter and coding region are the first intron of the maize ubiquitin (described in U.S. Pat. No. 6,054,574, which is incorporated herein by reference in its entirety) and the first intron of rice actin 1 (McElroy, Zhang et al. Plant Cell 2 (2): 163-171, 1990). Additional introns that are beneficial to gene expression when located between the promoter and coding region are the maize hsp70 intron (described in U.S. Pat. No. 5,859,347, which is incorporated herein by reference in its entirety), and the maize alcohol dehydrogenase 1 genes introns 2 and 6 (described in U.S. Pat. No. 6,342,660, which is incorporated herein by reference in its entirety).

In still other embodiments, transgenic plants are provided where the transgene that provides for MSH1 and DRM2 gene suppression is flanked by sequences that provide for removal for the transgene. Such sequences include, but are not limited to, transposable element sequences that are acted on by a cognate transposase. Non-limiting examples of such systems that have been used in transgenic plants include the cre-lox and FLP-FRT systems.

MSH1 and DRM2 gene suppression can be readily identified or monitored by molecular techniques. In certain embodiments where the endogenous MSH1 and DRM2 gene is intact but its expression is inhibited, production or accumulation of the RNA encoding MSH1 and DRM2 gene can be monitored. Molecular methods for monitoring MSH1 and DRM2 gene RNA expression levels include, but are not limited to, use of semi-quantitative or quantitative reverse transcriptase polymerase chain reaction (qRT-PCR) techniques. The use of semi-quantitative PCR techniques to monitor MSH1 and DRM2 gene suppression resulting from RNAi mediated suppression of MSH1 and DRM2 gene has been described (Sandhu et al. 2007). Various quantitative RT-PCR procedures including, but not limited to, TaqMan™ reactions (Applied Biosystems, Foster City, Calif. US), use of Scorpion™ or Molecular Beacon™ probes, or any of the methods disclosed in Bustin, S. A. (Journal of Molecular Endocrinology (2002) 29, 23-39) can be used. It is also possible to use other RNA quantitation techniques such as Quantitative Nucleic Acid Sequence Based Amplification (Q-NASBA™) or the Invader™ technology (Third Wave Technologies, Madison, Wis.).

In certain embodiments where MSH1 and DRM2 gene suppression is achieved by use of a loss-of-function mutation in the endogenous MSH1 and DRM2 gene of a plant, the presence or absence of that loss-of-function mutation in the genomic DNA can be readily determined by a variety of techniques. Certain techniques can also be used that provide for identification of the mutation in a hemizygous state (i.e. where one chromosome carries the mutated msh1 gene and the other chromosome carries the wild type MSH1 and DRM2 gene gene). Mutations in MSH1 and DRM2 DNA sequences that include insertions, deletions, nucleotide substitutions, and combinations thereof can be detected by a variety of effective methods including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981 and 7,250,252 all of which are incorporated herein by reference in their entireties. For example, mutations can be detected by hybridization to allele-specific oligonucleotide (ASO) probes as disclosed in U.S. Pat. Nos. 5,468,613 and 5,217,863. U.S. Pat. No. 5,210,015 discloses detection of annealed oligonucleotides where a 5′ labelled nucleotide that is not annealed is released by the 5′-3′ exonuclease activity. U.S. Pat. No. 6,004,744 discloses detection of the presence or absence of mutations in DNA through a DNA primer extension reaction. U.S. Pat. No. 5,468,613 discloses allele specific oligonucleotide hybridizations where single or multiple nucleotide variations in nucleic acid sequence can be detected by a process in which the sequence containing the nucleotide variation is amplified, affixed to a support and exposed to a labeled sequence-specific oligonucleotide probe. Mutations can also be detected by probe ligation methods as disclosed in U.S. Pat. No. 5,800,944 where sequence of interest is amplified and hybridized to probes followed by ligation to detect a labeled part of the probe. U.S. Pat. Nos. 6,613,509 and 6,503,710, and references found therein provide methods for identifying mutations with mass spectroscopy. These various methods of identifying mutations are intended to be exemplary rather than limiting as the methods of the present invention can be used in conjunction with any polymorphism typing method to identify the presence of absence of mutations in an MSH1 and DRM2 gene in genomic DNA samples. Furthermore, genomic DNA samples used can include, but are not limited to, genomic DNA isolated directly from a plant, cloned genomic DNA, or amplified genomic DNA.

Loss-of-function mutations in endogenous plant MSH1 and DRM2 genes, including MSH1 and DRM2 genes encoding the cDNAs provided in Table 1, can be obtained from a variety of sources and by a variety of techniques. A homologous replacement sequence containing one or more loss of function mutations in the MSH1 and DRM2 gene and homologous sequences at both ends of the double stranded break can provide for homologous recombination and substitution of the resident wild-type MSH1 and/or DRM2 gene sequence in the chromosome with a msh1 and/or drm2 replacement sequence with the loss of function mutation(s). Such loss of function mutations include, but are not limited to, insertions, deletions, and substitutions of sequences within an MSH1 and DRM2 gene that result in either a complete loss of MSH1 and DRM2 gene function or a loss of MSH1 and DRM2 gene function sufficient to elicit alterations (i.e. heritable and reversible epigenetic changes) in other chromosomal loci or mutations in other chromosomal loci. Loss-of-function mutations in MSH1 and DRM2 gene include, but are not limited to, frameshift mutations, pre-mature translational stop codon insertions, deletions of one or more functional domains that for MSH1 include, but are not limited to, a DNA binding (Domain I), an ATPase (Domain V) domain, and/or a carboxy-terminal GIY-YIG type endonuclease domain, and the like. Also provided herein are mutations analogous the Arabidopsis msh1 mutation that are engineered into endogenous MSH1 plant gene to obtain similar effects. Methods for substituting endogenous chromosomal sequences by homologous double stranded break repair have been reported in tobacco and maize (Wright et al., Plant J. 44, 693, 2005; D'Halluin, et al., Plant Biotech. J. 6:93, 2008). A homologous replacement msh1 or drm2 sequence comprising a loss-of-function mutation (i.e. which provides a loss of function mutation in an MSH1 or DRM2 target gene sequence) can also be introduced into a targeted nuclease cleavage site by non-homologous end joining or a combination of non-homologous end joining and homologous recombination (reviewed in Puchta, J. Exp. Bot. 56, 1, 2005; Wright et al., Plant J. 44, 693, 2005). In certain embodiments, at least one site specific double stranded break can be introduced into the endogenous MSH1 and DRM2 target gene by a meganuclease. Genetic modification of meganucleases can provide for meganucleases that cut within a recognition sequence that exactly matches or is closely related to specific endogenous target gene sequence (WO/06097853A1, WO/06097784A1, WO/04067736A2, U.S. Pat. No. 20070117128A1). Methods for introduction of the loss-of-function mutations in MSH1 and DRM2 thus includes use of site-specific nucleases including meganucleases, zinc finger nucleases, transcription activator-like effector nucleases (TALENS), clustered regularly interspaced short palindromic repeat (CRISPR)-associated Cas nuclease (e.g., Cas9, Cas12a, Cms1, S. aureus Cas9 variants, a Cas9, a nCas9 nickase, a type V Cas nuclease, a Cas12a nuclease, a nCas12a nickase, a Cas12d (CasY), a Cas12e (CasX), a Cas12b (C2c1), a Cas12c (C2c3), a Cas12i, a Cas12f, a Cas12j, a Cas14, or eSpCas9 nuclease) in combination with guide RNAs, and the like. Methods where these site-specific nucleases, and in particular, CRISPR/Cas systems comprising a Cas nuclease and a guide RNA directed to MSH1 and DRM2 are contemplated. Cpf1 or Csm1 nucleases are disclosed in US Patent Application Publication 20180148735, which is incorporated herein by reference in its entirety, and can be used to obtain MSH1 and DRM2 loss-of-function mutations. CRISPR-Cas systems disclosed in US Patent Application Publications 20150344912, 20160138008, 20180179547, 20200172886, and 20220282244, which are incorporated herein by reference in its entirety, can also be used to obtain MSH1 and DRM2 loss-of-function mutations. It is thus anticipated that one can select or design a nuclease that will cut within an MSH1 or DRM2 gene sequence which can result in a loss-of-function mutation through non-homologous end joining (NHEJ)-mediated repair. In other embodiments, at least one site specific double stranded break can be introduced in the endogenous MSH1 and/or DRM2 target gene target sequence with a zinc finger nuclease. The use of engineered zinc finger nuclease to provide homologous recombination in plants has also been disclosed (WO 03/080809, WO 05/014791, WO 07014275, WO 08/021207). In still other embodiments, mutations in endogenous MSH1 and/or DRM2 target gene genes can be identified through use of the TILLING technology (Targeting Induced Local Lesions in Genomes) as described by Henikoff et al. where traditional chemical mutagenesis would be followed by high-throughput screening to identify plants comprising point mutations or other mutations in the endogenous MSH1 and/or DRM2 target gene (Henikoff et al., Plant Physiol. 2004, 135:630-636). The recovery of mutations in endogenous MSH1 and DRM2 genes is specifically provided herein. In certain embodiments where crop plants comprise two or three MSH1 and DRM2 genes (e.g., certain crop plants provided in Table 1), loss-of-function mutations can be introduced in 1, 2, or, if applicable, 3 of the MSH1 and/or DRM2 genes present in the crop plant. In certain embodiments where a crop plant (e.g., certain crop plants provided in Table 1) has an MSH1 and/or DRM2 gene which gives rise to multiple transcripts (e.g., undergoes alternative splicing), loss-of function mutations comprising deletions and/or frameshift mutations located near the 5′ end of the MSH1 and/or DRM2 coding region can be introduced in the MSH1 and/or DRM2 gene.

Any of the recombinant DNA constructs provided herein can be introduced into the chromosomes of a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation. Aforementioned methods of introducing transgenes are well known to those skilled in the art and are described in U.S. patent application No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton), each of which are incorporated herein by reference in their entirety. Methods of using bacteria such as Rhizobium or Sinorhizobium to transform plants are described in Broothaerts, et al., Nature. 2005, 10; 433 (7026): 629-33. It is further understood that the recombinant DNA constructs can comprise cis-acting site-specific recombination sites recognized by site-specific recombinases, including Cre, Flp, Gin, Pin, Sre, pinD, Int-B13, and R. Methods of integrating DNA molecules at specific locations in the genomes of transgenic plants through use of site-specific recombinases can then be used (U.S. Pat. No. 7,102,055). Those skilled in the art will further appreciate that any of these gene transfer techniques can be used to introduce the recombinant DNA constructs into the chromosome of a plant cell, a plant tissue or a plant.

Methods of introducing plant minichromosomes comprising plant centromeres that provide for the maintenance of the recombinant minichromosome in a transgenic plant can also be used in practicing this invention (U.S. Pat. No. 6,972,197 and U.S. Patent Application Publication 20120047609). In these embodiments of the invention, the transgenic plants harbor the minichromosomes as extrachromosomal elements that are not integrated into the chromosomes of the host plant. It is anticipated that such mini-chromosomes may be useful in providing for variable transmission of a resident recombinant DNA construct that suppresses expression of an MSH1 and/or DRM2 target gene.

Methods where MSH1 and DRM2 gene suppression is effected in cultured plant cells are also provided herein. In certain embodiments, MSH1 and DRM2 gene suppression is effected in cultured plant cells by introducing a nucleic acid that provides for such suppression into the plant cells. Nucleic acids that can be used to provide for suppression of MSH1 and DRM2 gene in cultured plant cells include, but are not limited to, transgenes that produce a small inhibitory RNA (siRNA), a microRNA (miRNA), a co-suppressing sense RNA, and/or an anti-sense RNA directed to the MSH1 and DRM2 gene. Nucleic acids that can be used to provide for suppression of MSH1 and DRM2 gene in cultured plant cells include, but are not limited to, a small inhibitory RNA (siRNA) or a microRNA (miRNA) directed against the endogenous MSH1 and DRM2 gene. RNA molecules that provide for inhibition of MSH1 and DRM2 gene can be introduced by electroporation. Introduction of inhibitory RNAs to cultured plant cells to inhibit target genes can in certain embodiments be accomplished as disclosed in Vanitharani et al. (Proc Natl Acad Sci USA., 2003, 100 (16): 9632-6), Qi et al. (Nucleic Acids Res. 2004 Dec. 15; 32 (22): e179), or J. Cheon et al. (Microbiol. Biotechnol. (2009), 19 (8), 781-786).

Methods where MSH1 and DRM2 gene suppression is effected in vegetatively or clonally propagated plant materials are also provided herein. Such vegetatively or clonally propagated plant materials can include, but are not limited to, cuttings, cultured plant materials, and the like. In certain embodiments, recovery of such plant or clonally propagated plant materials that have been subjected to MSH1 and DRM2 gene suppression can be accomplished by methods that allow for transient suppression of the MSH1 and DRM2 gene. In certain non-limiting examples, plant or clonally propagated plant materials that have been subjected to plant MSH1 and DRM2 gene suppression are recovered by placing recombinant DNA constructs that suppress a MSH1 and DRM2 gene in vectors that provide for their excision or segregation. In certain embodiments, such excision can be facilitated by use of transposase-based systems or such segregation can be facilitated by use of mini-chromosomes. In certain embodiments, such excision or segregation can be facilitated by linking a transgene that provides for a “conditional-lethal” counter selection to the transgene that suppresses a MSH1 and DRM2 in the recombinant DNA construct. Vegetatively or clonally propagated plant materials that have been subjected to MSH1 and DRM2 gene suppression and lacking recombinant DNA constructs that suppress a MSH1 and DRM2 gene can then be screened and/or selected for useful traits. Also provided are methods where vegetatively or clonally propagated plant materials are obtained from a plant resulting from a self or outcross or from a cultured plant cell, where either the plant or plant cell had been subjected to suppression of a MSH1 and DRM2 gene. Such vegetatively or clonally propagated plant materials obtained from such plants resulting from a self or outcross or from a plant cell that have been subjected to MSH1 and DRM2 gene suppression can also be screened and/or selected for useful traits. Also provided herein are methods where a sexually reproducing plant or plant population comprising useful traits is vegetatively or clonally propagated, and a plant or a plant population derived therefrom is then used to produce seed or a seed lot.

MSH1 and DRM2 gene suppression can also be readily identified or monitored by traditional methods where plant phenotypes are observed. For example, MSH1 and DRM2 gene suppression can be identified or monitored by observing organellar effects that include leaf variegation, cytoplasmic male sterility (CMS), a reduced growth-rate phenotype, and/or delayed or non-flowering phenotype. Phenotypes indicative of MSH1 gene suppression in various plants are provided in WO 2012/151254, which is incorporated herein by reference in its entirety. These phenotypes that are associated with MSH1 gene suppression are referred to herein as “discrete variation” (V_{D}). MSH1 gene suppression can also produce changes in plant phenotypes including, but not limited to, plant tillering, height, internode elongation and stomatal density (referred to herein as “MSH1-dr”) that can be used to identify or monitor MSH1 gene suppression in plants. Other biochemical and molecular traits can also be used to identify or monitor MSH1 gene suppression in plants. Such molecular traits can include, but are not limited to, changes in expression of genes involved in cell cycle regulation, Gibberellic acid catabolism auxin biosynthesis, auxin receptor expression, flower and vernalization regulators (i.e. increased FLC and decreased SOC/expression), as well as increased miR156 and decreased miR172 levels. Such biochemical traits can include, but are not limited to, up-regulation of most compounds of the TCA, NAD and carbohydrate metabolic pathways, down-regulation of amino acid biosynthesis, depletion of sucrose in certain plants, increases in sugars or sugar alcohols in certain plants, as well as increases in ascorbate, alphatocopherols, and stress-responsive flavones apigenin, and apigenin-7-oglucoside, isovitexin, kaempferol 3-O-beta-glucoside, luteolin-7-O-glucoside, and vitexin. In certain embodiments, elevated plastochromanol-8 levels in plant stems can serve as a biochemical marker that can be used to identify or monitor MSH1 gene suppression. In particular, plastochromanol-8 levels in stems of plants subjected to MSH1 gene suppression can be compared to the levels in control plants that have not been subjected to such suppression to identify or monitor MSH1 and DRM2 target gene suppression. It is further contemplated that in certain embodiments, a combination of both molecular, biochemical, and traditional methods can be used to identify or monitor MSH1 and DRM2 gene suppression in plants.

Plants or rootstocks subjected to MSH1 and DRM2 gene suppression, and scions grafted to such rootstocks, as well as the progeny thereof, can exhibit a variety of nuclear chromosomal DNA methylation patterns that are absent from control plants, rootstocks, or scions that were not subjected to MSH1 and DRM2 gene suppression. Such methylation patterns can include, but are not limited to, CG hypermethylation, pericentromeric CHG hypermethylation, and/or additional characteristic methylation patterns observed in plants or progeny thereof that had been subjected to suppression of MSH1 and DRM2 gene expression. Such methylation patterns can also include, but are not limited to, changes in 5-hydroxymethylation and in particular, the occurrence of 5-hydroxymethylcytosine (5-hmC). Changes in 5-hmC can be monitored by immunoassays (Quest 5-hmC™ DNA ELISA Kit, Zymo Research Corp., Irvine, Calif., USA; or EpiSeeker™ hydroxymethylated DNA Quantification Kit, Abeam, Inc., Cambridge, Mass.). It is anticipated that plants, plant parts, processed plant products, rootstocks, and scions provided herein or produced by the methods provided herein can be identified by comparing methylation patterns in the genomic DNA of such materials to the methylation patterns of control plants, plant parts, processed plant products, rootstocks, and scions.

In certain embodiments of the methods provided herein, progeny plants derived from plants where MSH1 and DRM2 gene expression was suppressed that exhibit male sterility, dwarfing, variegation, and/or delayed flowering time and express functional MSH1 and DRM2 genes are obtained and maintained as independent breeding lines or as populations of plants. It has been found that such phenotypes appear to sort, so that it is feasible to select a cytoplasmic male sterile plant displaying normal growth rate and no variegation, for example, or a stunted, male fertile plant that is highly variegated. We refer to this phenomenon herein as discrete variation (V_{D}). Exemplary and non-limiting illustrations of this phenomenon as it occurs in selfed plant populations that have lost an MSH1 gene-inhibiting transgene by segregation have been disclosed (WO 2012/151254, incorporated herein by reference in its entirety). It is further contemplated that such individual lines that exhibit discrete variation (V_{D}) can be obtained by any of the aforementioned genetic techniques, molecular genetic techniques, or combinations thereof.

Individual lines obtained from plants where MSH1 and DRM2 target gene expression was suppressed that exhibit discrete variation (V_{D}) can be crossed to other plants to obtain progeny plants that lack the phenotypes associated with discrete variation (V_{D}) (i.e. male sterility, dwarfing, variegation, and/or delayed flowering time). In certain embodiments, progeny of such outcrosses can be selfed to obtain individual progeny lines that exhibit significant phenotypic variation. Such phenotypic variation that is observed in these individual progeny lines derived from outcrosses of plants where MSH1 and DSM2 gene expression was suppressed and that exhibit discrete variation to other plants is herein referred to as “quantitative variation” (VQ). Certain individual progeny plant lines obtained from the outcrosses of plants where MSH1 gene expression was suppressed to other plants can exhibit useful phenotypic variation where one or more traits are improved relative to either parental line and can be selected. Useful phenotypic variation that can be selected in such individual progeny lines includes, but is not limited to, increases in fresh and dry weight biomass relative to either parental line. An exemplary and non-limiting illustration of this phenomenon as it occurs in F2 progeny of outcrosses of plants that exhibit discrete variation to plants that do not exhibit discrete variation is provided in WO 2012/151254, which is incorporated herein by reference in its entirety.

Individual lines obtained from plants where MSH1 and DRM2 gene expression was suppressed that exhibit discrete variation (V_{D}) can also be selfed to obtain progeny plants that lack the phenotypes associated with discrete variation (V_{D}) (i.e. male sterility, dwarfing, variegation, and/or delayed flowering time). Recovery of such progeny plants that lack the undesirable phenotypes can in certain embodiments be facilitated by removal of the transgene or endogenous locus that provides for MSH1 and DRM2 gene suppression. In certain embodiments, progeny of such selfs can be used to obtain individual progeny lines or populations that exhibit significant phenotypic variation. Certain individual progeny plant lines or populations obtained from selfing plants where MSH1 and DRM2 gene expression was suppressed can exhibit useful phenotypic variation where one or more traits are improved relative to the parental line that was not subjected to MSH1 and DRM2 gene suppression and can be selected. Useful phenotypic variation that can be selected in such individual progeny lines includes, but is not limited to, increases in fresh and dry weight biomass relative to the parental line.

In certain embodiments, an outcross of an individual line exhibiting discrete variability can be to a plant that has not been subjected to MSH1 and DRM2 gene suppression but is otherwise isogenic to the individual line exhibiting discrete variation. In certain exemplary embodiments, a line exhibiting discrete variation is obtained by suppressing MSH1 and DRM2 gene in a given germplasm and can outcrossed to a plant having that same germplasm that was not subjected to MSH1 and DRM2 gene suppression. In other embodiments, an outcross of an individual line exhibiting discrete variability can be to a plant that has not been subjected to MSH1 and DRM2 gene suppression but is not isogenic to the individual line exhibiting discrete variation. Thus, in certain embodiments, an outcross of an individual line exhibiting discrete variability can also be to a plant that comprises one or more chromosomal polymorphisms that do not occur in the individual line exhibiting discrete variability, to a plant derived from partially or wholly different germplasm, or to a plant of a different heterotic group (in instances where such distinct heterotic groups exist). It is also recognized that such an outcross can be made in either direction. Thus, an individual line exhibiting discrete variability can be used as either a pollen donor or a pollen recipient to a plant that has not been subjected to MSH1 and DRM2 gene suppression in such outcrosses. In certain embodiments, the progeny of the outcross are then selfed to establish individual lines that can be separately screened to identify lines with improved traits relative to parental lines. Such individual lines that exhibit the improved traits are then selected and can be propagated by further selfing. An exemplary and non-limiting illustration of this procedure where F2 progeny of outcrosses of plants that exhibit discrete variation to plants that do not exhibit discrete variation are obtained is provided in WO 2012/151254, which is incorporated herein by reference in its entirety. Such F2 progeny lines are screened for desired trait improvements relative to the parental plants and lines exhibiting such improvements are selected.

In certain embodiments, sub-populations of plants comprising the useful traits and epigenetic changes induced by suppression of the MSH1 and DRM2 gene can be selected and bred as a population. Such populations can then be subjected to one or more additional rounds of selection for the useful traits and/or epigenetic changes to obtain subsequent sub-populations of plants exhibiting the useful trait. Any of these sub-populations can also be used to generate a seed lot. In an exemplary embodiment, plastid perturbed plants exhibiting an Msh1-dr phenotype can be selfed or outcrossed to obtain an F1 generation. A bulk selection at the F1, F2, and/or F3 generation can thus provide a population of plants exhibiting the useful trait and/or epigenetic changes or a seed lot. In certain embodiments, it is also anticipated that populations of progeny plants or progeny seed lots comprising a mixture of inbred an hybrid germplasms can be derived from populations comprising hybrid germplasm (i.e. plants arising from cross of one inbred line to a distinct inbred line). In certain embodiments, such sub-populations can comprise grafted plants comprising a scion grafted to rootstock that had been subjected to MSH1 and DRM2 gene suppression. Sub-populations of grafted plants where the rootstock source plant is the progeny of a parental plant that had been subjected to MSH1 and DRM2 gene suppression and that was selected for one or more useful traits can also be selected and bred as a population. Any of the aforementioned subpopulations can comprise 2 or more, 10 or more, 50 or more, 100 or more, 1000 or more, or 10,000 or more plants. Seed lots thus obtained from these exemplary method or other methods provided herein can comprise seed wherein at least 25%, 50%, 60%, 70%, 80%, 90%, or 95% of progeny plants grown from the seed exhibit a useful trait. The selection would provide the most robust and vigorous of the population for seed lot production. Seed lots produced in this manner could be used for either breeding or sale. In certain embodiments, a seed lot comprising seed wherein at least 25%, 50%, 60%, 70%, 80%, 90%, or 95% of progeny plants grown from the seed exhibit a useful trait associated with one or more epigenetic changes, wherein the epigenetic changes are associated with CG hyper-methylation and/or CHG hyper-methylation at one or more nuclear chromosomal loci in comparison to a control plant that does not exhibit the useful trait, and wherein the seed or progeny plants grown from said seed that is epigenetically heterogenous are obtained. A seed lot obtainable by these methods can include at least 100, 500, 1000, 5000, or 10,000 seeds.

In certain embodiments, methods for producing a seed lot comprising: (i) growing a population of plants, wherein said population comprises two or more of grafted plants comprising a scion and rootstock obtained from a plant that had been subjected to MSH1 and DRM2 gene suppression, or from a parental plant that had been subjected to MSH1 and DRM2 gene suppression; and (ii) obtaining a seed lot from the population are provided. Populations of grafted plants where the rootstock source plant is the progeny of a parental plant that had been subjected to MSH1 and DRM2 gene suppression and that was selected for one or more useful traits can also be selected and bred as a population. Any of the aforementioned populations can comprise 2 or more, 10 or more, 50 or more, 100 or more, 1000 or more, or 10,000 or more plants. Seed lots thus obtained from these exemplary methods or other methods provided herein can comprise seed wherein at least 25%, 50%, 60%, 70%, 80%, 90%, or 95% of progeny plants grown from the seed exhibit a useful trait. The selection would provide the most robust and vigorous of the population for seed lot production. Seed lots produced in this manner could be used for either breeding or sale. In certain embodiments, a seed lot comprising seed wherein at least 25%, 50%, 60%, 70%, 80%, 90%, or 95% of progeny plants grown from the seed exhibit a useful trait associated with one or more epigenetic changes, wherein the epigenetic changes are associated with CG hyper-methylation and/or CHG hyper-methylation at one or more nuclear chromosomal loci in comparison to corresponding nuclear chromosomal loci of a control plant that does not exhibit the useful trait, and wherein the seed or progeny plants grown from said seed that is epigenetically heterogenous are obtained. A seed lot obtainable by these methods can include at least 100, 500, 1000, 5000, or 10,000 seeds.

Altered chromosomal loci that can confer useful traits can also be identified and selected by performing appropriate comparative analyses of reference plants that do not exhibit the useful traits and test plants obtained from a parental plant or plant cell that had been subjected to MSH1 and DRM2 gene suppression and obtaining either the altered loci or plants comprising the altered loci. It is anticipated that a variety of reference plants and test plants can be used in such comparisons and selections. In certain embodiments, the reference plants that do not exhibit the useful trait include, but are not limited to, any of: a) a wild-type plant; b) a distinct subpopulation of plants within a given F2 population of plants of a given plant line (where the F2 population is any applicable plant type or variety); c) an F1 population exhibiting a wild type phenotype (where the F1 population is any applicable plant type or variety); and/or, d) a plant that is isogenic to the parent plants or parental cells of the test plants prior to suppression of MSH1 and DRM2 gene in those parental plants or plant cells (i.e. the reference plant is isogenic to the plants or plant cells that were later subjected to MSH1 and DRM2 gene suppression to obtain the test plants). In certain embodiments, the test plants that exhibit the useful trait include, but are not limited to, any of: a) any non-transgenic segregants that exhibit the useful trait and that were derived from parental plants or plant cells that had been subjected to transgene mediated MSH1 and DRM2 gene suppression, b) a distinct subpopulation of plants within a given F2 population of plants of a given plant line that exhibit the useful trait (where the F2 population is any applicable plant type or variety); (c) any progeny plants obtained from the plants of (a) or (b) that exhibit the useful trait; or d) a plant or plant cell that had been subjected to MSH1 and DRM2 gene suppression that exhibit the useful trait.

In general, an objective of these comparisons is to identify differences in the small RNA profiles and/or methylation of certain chromosomal DNA loci between test plants that exhibit the useful traits and reference plants that do not exhibit the useful traits. Altered loci thus identified can then be isolated or selected in plants to obtain plants exhibiting the useful traits.

In certain embodiments, altered chromosomal loci can be identified by identifying small RNAs that are up or down regulated in the test plants (in comparison to reference plants). This method is based in part on identification of altered chromosomal loci where small interfering RNAs direct the methylation of specific gene targets by RNA-directed DNA methylation (RdDM). The RNA-directed DNA methylation (RdDM) process has been described (Chinnusamy V et al. Sci China Ser C-Life Sci. (2009) 52 (4): 331-343). Any applicable technology platform can be used to compare small RNAs in the test and reference plants, including, but not limited to, microarray-based methods (Franco-Zorilla et al. Plant J. 2009 59(5): 840-50), deep sequencing based methods (Wang et al. The Plant Cell 21:1053-1069 (2009)), and the like.

In certain embodiments, altered chromosomal loci can be identified by identifying histone proteins associated with a locus and that are methylated or acylated in the test plants (in comparison to reference plants). The analysis of chromosomal loci associated with methylated or acylated histones can be accomplished by enriching and sequencing those loci using antibodies that recognize methylated or acylated histones. Identification of chromosomal regions associated with methylation or acetylation of specific lysine residues of histone H3 by using antibodies specific for H3K4me3, H3K9ac, H3K27me3, and H3K36me3 has been described (Li et al., Plant Cell 20:259-276, 2008; Wang et al. The Plant Cell 21:1053-1069 (2009).

In certain embodiments, altered chromosomal loci can be identified by identifying chromosomal regions (genomic DNA) that has an altered methylation status in the test plants (in comparison to reference plants). An altered methylation status can comprise either the presence or absence of methylation in one or more chromosomal loci of a test plant comparison to a reference plant. Any applicable technology platform can be used to compare the methylation status of chromosomal loci in the test and reference plants. Applicable technologies for identifying chromosomal loci with changes in their methylation status include, but not limited to, methods based on immunoprecipitation of DNA with antibodies that recognize 5-methylcytidine, methods based on use of methylation dependent restriction endonucleases and PCR such as McrBC-PCR methods (Rabinowicz, et al. Genome Res. 13:2658-2664 2003; Li et al., Plant Cell 20:259-276, 2008), sequencing of bisulfite-converted DNA (Frommer et al. Proc. Natl. Acad. Sci. U.S.A. 89 (5): 1827-31; Tost et al. BioTechniques 35 (1): 152-156, 2003), methylation-specific PCR analysis of bisulfite treated DNA (Herman et al. Proc. Natl. Acad. Sci. U.S.A. 93 (18): 9821-6, 1996), deep sequencing based methods (Wang et al. The Plant Cell 21:1053-1069 (2009)), methylation sensitive single nucleotide primer extension (MsSnuPE; Gonzalgo and Jones Nucleic Acids Res. 25 (12): 2529-2531, 1997), fluorescence correlation spectroscopy (Umezu et al. Anal Biochem. 415 (2): 145-50, 2011), single molecule real time sequencing methods (Flusberg et al. Nature Methods 7, 461-465), high resolution melting analysis (Wojdacz and Dobrovic (2007) Nucleic Acids Res. 35 (6): e41), and the like.

Methods for introducing various chromosomal modifications that can confer a useful trait into a plant, as well as the plants, plant parts, and products of those plant parts are also provided herein. Chromosomal alterations and/or chromosomal mutations induced by suppression of MSH1 and DRM2 gene can be identified as described herein. Once identified, chromosomal modifications including, but not limited to, chromosomal alterations, chromosomal mutations, or transgenes that provide for the same genetic effect as the chromosomal alterations and/or chromosomal mutations induced by suppression of MSH1 and DRM2 gene can be introduced into host plants to obtain plants that exhibit the desired trait. In this context, the “same genetic effect” means that the introduced chromosomal modification provides for an increase and/or a reduction in expression of one or more endogenous plant genes that is similar to that observed in a plant that has been subjected to MSH1 and DRM2 gene suppression and exhibits the useful trait. In certain embodiments where an endogenous gene is methylated in a plant subjected to MSH1 and DRM2 gene suppression and exhibits both reduced expression of that gene and a useful trait, chromosomal modifications in other plants that also result in reduced expression of that gene and the useful trait are provided. In certain embodiments where an endogenous gene is demethylated in a plant subjected to MSH1 and DRM2 gene suppression and exhibits both increased expression of that gene and a useful trait, chromosomal modifications in other plants that also result in increased expression of that gene and that useful trait are provided.

In certain embodiments, the chromosomal modification that is introduced is a chromosomal alteration. Chromosomal alterations including, but not limited to, a difference in a methylation state can be introduced by crossing a plant comprising the chromosomal alteration to a plant that lacks the chromosomal alteration and selecting for the presence of the alteration in F1, F2, or any subsequent generation progeny plants of the cross. In still other embodiments, the chromosomal alterations in specific target genes can be introduced by expression of a siRNA or hairpin RNA targeted to that gene by RNA directed DNA methylation (Chinnusamy V et al. Sci China Ser C-Life Sci. (2009) 52 (4): 331-343; Cigan et al. Plant J 43 929-940, 2005; Heilersig et al. (2006) Mol Genet Genomics 275 437-449; Miki and Shimamoto, Plant Journal 56 (4): 539-49; Okano et al. Plant Journal 53 (1): 65-77, 2008).

In certain embodiments, the chromosomal modification is a chromosomal mutation. Chromosomal mutations that provide for reductions or increases in expression of an endogenous gene of a chromosomal locus can include, but are not limited to, insertions, deletions, and/or substitutions of nucleotide sequences in a gene. Chromosomal mutations can result in decreased expression of a gene by a variety of mechanisms that include, but are not limited to, introduction of missense codons, frame-shift mutations, premature translational stop codons, promoter deletions, mutations that disrupt mRNA processing, and the like. Chromosomal mutations that result in increased expression of a gene include, but are not limited to, promoter substitutions, removal of negative regulatory elements from the gene, and the like. Chromosomal mutations can be introduced into specific loci of a plant by any applicable method. Applicable methods for introducing chromosomal mutations in endogenous plant chromosomal loci include, but are not limited to, homologous double stranded break repair (Wright et al., Plant J. 44, 693, 2005; D'Halluin, et al., Plant Biotech. J. 6:93, 2008), non-homologous end joining or a combination of non-homologous end joining and homologous recombination (reviewed in Puchta, J. Exp. Bot. 56, 1, 2005; Wright et al., Plant J. 44, 693, 2005), meganuclease-induced, site specific double stranded break repair (WO/06097853A1, WO/06097784A1, WO/04067736A2, U.S. Pat. No. 20070117128A1), and zinc finger nuclease mediated homologous recombination (WO 03/080809, WO 05/014791, WO 07014275, WO 08/021207). In still other embodiments, desired mutations in endogenous plant chromosomal loci can be identified through use of the TILLING technology (Targeting Induced Local Lesions in Genomes) as described (Henikoff et al., Plant Physiol. 2004, 135:630-636).

In other embodiments, chromosomal modifications that provide for the desired genetic effect can comprise a transgene. Transgenes that can result in decreased expression of an gene by a variety of mechanisms that include, but are not limited to, dominant-negative mutants, a small inhibitory RNA (siRNA), a microRNA (miRNA), a co-suppressing sense RNA, and/or an anti-sense RNA and the like. U.S. patents incorporated herein by reference in their entireties that describe suppression of endogenous plant genes by transgenes include U.S. Pat. Nos. 7,109,393, 5,231,020 and 5,283,184 (co-suppression methods); and U.S. Pat. Nos. 5,107,065 and 5,759,829 (antisense methods). In certain embodiments, transgenes specifically designed to produce double-stranded RNA (dsRNA) molecules with homology to the endogenous gene of a chromosomal locus can be used to decrease expression of that endogenous gene. In such embodiments, the sense strand sequences of the dsRNA can be separated from the antisense sequences by a spacer sequence, preferably one that promotes the formation of a dsRNA (double-stranded RNA) molecule. Examples of such spacer sequences include, but are not limited to, those set forth in Wesley et al., Plant J., 27 (6): 581-90 (2001), and Hamilton et al., Plant J., 15:737-746 (1998). Vectors for inhibiting endogenous plant genes with transgene-mediated expression of hairpin RNAs are disclosed in U.S. patents application Nos. 20050164394, 20050160490, and 20040231016, each of which is incorporated herein by reference in their entirety.

Transgenes that result in increased expression of a gene of a chromosomal locus include, but are not limited to, a recombinant gene fused to heterologous promoters that are stronger than the native promoter, a recombinant gene comprising elements such as heterologous introns, 5′ untranslated regions, 3′ untranslated regions that provide for increased expression, and combinations thereof. Such promoter, intron, 5′ untranslated, 3′ untranslated regions, and any necessary polyadenylation regions can be operably linked to the DNA of interest in recombinant DNA molecules that comprise parts of transgenes useful for making chromosomal modifications as provided herein.

Exemplary promoters useful for expression of transgenes include, but are not limited to, enhanced or duplicate versions of the viral CaMV35S and FMV35S promoters (U.S. Pat. No. 5,378,619, incorporated herein by reference in its entirety), the cauliflower mosaic virus (CaMV) 19S promoters, the rice Act1 promoter and the Figwort Mosaic Virus (FMV) 35S promoter (U.S. Pat. No. 5,463,175; incorporated herein by reference in its entirety). Exemplary introns useful for transgene expression include, but are not limited to, the maize hsp70 intron (U.S. Pat. No. 5,424,412; incorporated by reference herein in its entirety), the rice Act1 intron (McElroy et al., 1990, The Plant Cell, Vol. 2, 163-171), the CAT-1 intron (Cazzonnelli and Velten, Plant Molecular Biology Reporter 21:271-280, September 2003), the pKANNIBAL intron (Wesley et al., Plant J. 2001 27 (6): 581-90; Collier et al., 2005, Plant J 43:449-457), the PIV2 intron (Mankin et al. (1997) Plant Mol. Biol. Rep. 15 (2): 186-196) and the “Super Ubiquitin” intron (U.S. Pat. No. 6,596,925, incorporated herein by reference in its entirety; Collier et al., 2005, Plant J 43:449-457). Exemplary polyadenylation sequences include, but are not limited to, and Agrobacterium tumor-inducing (Ti) plasmid nopaline synthase (NOS) gene and the pea ssRUBISCO E9 gene polyadenylation sequences.

Plant lines and plant populations obtained by the methods provided herein can be screened and selected for a variety of useful traits by using a wide variety of techniques. In particular embodiments provided herein, individual progeny plant lines or populations of plants obtained from the selfs or outcrosses of plants where MSH1 and DRM2 gene expression was suppressed to other plants are screened and selected for the desired useful traits.

In certain embodiments, the screened and selected trait is improved plant yield. In certain embodiments, such yield improvements are improvements in the yield of a plant line relative to one or more parental line(s) under non-stress conditions. Non-stress conditions comprise conditions where water, temperature, nutrients, minerals, and light fall within typical ranges for cultivation of the plant species. Such typical ranges for cultivation comprise amounts or values of water, temperature, nutrients, minerals, and/or light that are neither insufficient nor excessive. In certain embodiments, such yield improvements are improvements in the yield of a plant line relative to parental line(s) under abiotic stress conditions. Such abiotic stress conditions include, but are not limited to, conditions where water, temperature, nutrients, minerals, and/or light that are either insufficient or excessive. Abiotic stress conditions would thus include, but are not limited to, drought stress, osmotic stress, nitrogen stress, phosphorous stress, mineral stress, heat stress, cold stress, and/or light stress. In this context, mineral stress includes, but is not limited to, stress due to insufficient or excessive potassium, calcium, magnesium, iron, manganese, copper, zinc, boron, aluminum, or silicon. In this context, mineral stress includes, but is not limited to, stress due to excessive amounts of heavy metals including, but not limited to, cadmium, copper, nickel, zinc, lead, and chromium.

Improvements in yield in plant lines obtained by the methods provided herein can be identified by direct measurements of wet or dry biomass including, but not limited to, grain, lint, leaves, stems, or seed. Improvements in yield can also be assessed by measuring yield related traits that include, but are not limited to, 100 seed weight, a harvest index, and seed weight. In certain embodiments, such yield improvements are improvements in the yield of a plant line relative to one or more parental line(s) and can be readily determined by growing plant lines obtained by the methods provided herein in parallel with the parental plants. In certain embodiments, field trials to determine differences in yield whereby plots of test and control plants are replicated, randomized, and controlled for variation can be employed (Giesbrecht F G and Gumpertz M L. 2004. Planning, Construction, and Statistical Analysis of Comparative Experiments. Wiley. New York; Mead, R. 1997. Design of plant breeding trials. In Statistical Methods for Plant Variety Evaluation. eds. Kempton and Fox. Chapman and Hall. London). Methods for spacing of the test plants (i.e. plants obtained with the methods of this invention) with check plants (parental or other controls) to obtain yield data suitable for comparisons are provided in references that include, but are not limited to, any of Cullis, B. et al. J. Agric. Biol. Env. Stat. 11:381-393; and Besag, J. and Kempton, R A. 1986. Biometrics 42:231-251).

In certain embodiments, the screened and selected trait is improved resistance to biotic plant stress relative to the parental lines. Biotic plant stress includes, but is not limited to, stress imposed by plant fungal pathogens, plant bacterial pathogens, plant viral pathogens, insects, nematodes, and herbivores. In certain embodiments, screening and selection of plant lines that exhibit resistance to fungal pathogens including, but not limited to, an Alternaria sp., an Ascochyta sp., a Botrytis sp.; a Cercospora sp., a Colletotrichum sp., a Diaporthe sp., a Diplodia sp., an Erysiphe sp., a Fusarium sp., Gaeumanomyces sp., Helminthosporium sp., Macrophomina sp., a Nectria sp., a Peronospora sp., a Phakopsora sp., Phialophora sp., a Phoma sp., a Phymatotrichum sp., a Phytophthora sp., a Plasmopara sp., a Puccinia sp., a Podosphaera sp., a Pyrenophora sp., a Pyricularia sp, a Pythium sp., a Rhizoctonia sp., a Scerotium sp., a Sclerotinia sp., a Septoria sp., a Thielaviopsis sp., an Uncinula sp, a Venturia sp., and a Verticillium sp. is provided. In certain embodiments, screening and selection of plant lines that exhibit resistance to bacterial pathogens including, but not limited to, an Erwinia sp., a Pseudomonas sp., and a Xanthamonas sp. is provided. In certain embodiments, screening and selection of plant lines that exhibit resistance to insects including, but not limited to, aphids and other piercing/sucking insects such as Lygus sp., lepidopteran insects such as Armigera sp., Helicoverpa sp., Heliothis sp., and Pseudoplusia sp., and coleopteran insects such as Diabroticus sp. is provided. In certain embodiments, screening and selection of plant lines that exhibit resistance to nematodes including, but not limited to, Meloidogyne sp., Heterodera sp., Belonolaimus sp., Ditylenchus sp., Globodera sp., Naccobbus sp., and Xiphinema sp. is provided.

Other useful traits that can be obtained by the methods provided herein include various seed quality traits including, but not limited to, improvements in either the compositions or amounts of oil, protein, or starch in the seed. Still other useful traits that can be obtained by methods provided herein include, but are not limited to, increased biomass, non-flowering, male sterility, digestibility, seed filling period, maturity (either earlier or later as desired), reduced lodging, and plant height (either increased or decreased as desired). Still other useful traits that can be obtained by methods provided herein include, but are not limited to, delayed leaf senescence, increased flower number, improved architecture for high density planting, improved photosynthesis, increased root mass, increased cell number, improved seedling vigor, improved seedling size, increased rate of cell division, improved metabolic efficiency, and increased meristem size.

In addition to any of the aforementioned traits, particularly useful traits for sorghum that can be obtained by the methods provided herein also include, but are not limited to: i) agronomic traits (flowering time, days to flower, days to flower-post rainy, days to flower-rainy; ii) fungal disease resistance (sorghum downy mildew resistance-glasshouse, sorghum downy mildew resistance-field, sorghum grain mold, sorghum leaf blight resistance, sorghum rust resistance; iii) grain related trait: (Grain dry weight, grain number, grain number per square meter, Grain weight over panicle, seed color, seed luster, seed size); iv) growth and development stage related traits (basal tillers number, days to harvest, days to maturity, nodal tillering, plant height, plant height-postrainy); v) inflorescence anatomy and morphology trait (threshability); vi) Insect damage resistance (sorghum shoot fly resistance-post-rainy, sorghum shoot fly resistance-rainy, sorghum stem borer resistance); vii) leaf related traits (leaf color, leaf midrib color, leaf vein color, flag leaf weight, leaf weight, rest of leaves weight); viii) mineral and ion content related traits (shoot potassium content, shoot sodium content); ix) panicle related traits (number of panicles, panicle compactness and shape, panicle exertion, panicle harvest index, panicle length, panicle weight, panicle weight without grain, panicle width); x) phytochemical compound content (plant pigmentation); xii) spikelet anatomy and morphology traits (glume color, glume covering); xiii) stem related trait (stem over leaf weight, stem weight); and xiv) miscellaneous traits (stover related traits, metabolized energy, nitrogen digestibility, organic matter digestibility, stover dry weight).

### Embodiments

This patent application includes, but is not limited to, the following embodiments.
1. A grafted plant comprising a scion to which a rootstock had been grafted, wherein:
(i) the scion is from a wild type plant;
(ii) MSH1 and DRM2 gene expression is suppressed in the rootstock;
(iii) the rootstock confers an improvement in yield or growth rate in progeny of the grafted plant in comparison to a control plant, wherein the control plant comprises either: (a) progeny of a scion grafted to rootstock that had not been subjected to suppression of MSH1 and DRM2 gene expression; (b) a whole plant that lacks any root graft and that had not been subjected to suppression of MSH1 and DRM2 gene expression; (c) a wild-type plant; or (d) progeny of a plant that is isogenic to the plant source of the scion of the grafted plant.
2. The grafted plant of embodiment 1, wherein the rootstock confers to the grafted plant an improvement in yield or growth rate in comparison to a control plant.
3. The grafted plant of embodiment 1 or 2, wherein the scion contains one or more epigenetic changes in one or more nuclear chromosomes, wherein the epigenetic changes are absent from the nuclear chromosomes of a control plant or are absent from nuclear chromosomes of a plant from which the scion was obtained.
4. The grafted plant of embodiment 3, wherein the epigenetic change(s) are also present in the rootstock.
5. The grafted plant of embodiment 3, wherein the epigenetic changes are associated with the improvement in the useful trait.
6. The grafted plant of embodiment 3, wherein the rootstock contain(s) one or more epigenetic changes in one or more nuclear chromosomes that are absent from nuclear chromosomes of rootstock obtained from a plant or nuclear chromosomes of a parent plant thereof that had not been subjected to suppression of MSH1 and DRM2 gene expression.
7. The grafted plant of any one of embodiments 1 to 6, wherein the MSH1 and DRM2 gene expression is suppressed in the rootstock by a loss-of-function mutation in an endogenous MSH1 and/or DRM2 gene of the rootstock and/or by a small inhibitory RNA (siRNA), a microRNA (miRNA), a co-suppressing sense RNA, and/or an anti-sense RNA having complementarity to the endogenous MSH1 and/or DRM2 gene promoter, 5′ or 3′ untranslated region, intron, coding region, and/or any combination thereof, optionally wherein the grafted plant is a soybean, Brassica, tomato, sorghum, rice, or corn plant and the endogenous MSH1 and DRM2 gene(s) encode the cDNA(s) set forth in Table 1.
8. The grafted plant of embodiment 7, wherein the MSH1 and DRM2 gene expression is suppressed in the rootstock by a loss-of-function mutation in an endogenous MSH1 and DRM2 gene(s), optionally wherein the grafted plant is a soybean, Brassica, tomato, sorghum, rice, or corn plant and the MSH1 and DRM2 gene(s) encode the cDNA(s) set forth in Table 1.
9. The grafted plant of embodiment 7, wherein the MSH1 and/or DRM2 gene expression is suppressed in the rootstock by a small inhibitory RNA (siRNA), a microRNA (miRNA), a co-suppressing sense RNA, and/or an anti-sense RNA having complementarity to the endogenous MSH1 and/or DRM2 gene promoter, 5′ or 3′ untranslated region, intron, coding region, and/or any combination thereof, optionally wherein the grafted plant is a soybean, Brassica, tomato, sorghum, rice, or corn plant and the MSH1 and DRM2 gene(s) encode the cDNA(s) set forth in Table 1.
10. The grafted plant of any one of embodiments 1 to 9, where said plant is selected from the group consisting of a crop plant, a tree, a bush, turf grass, pasture grass, and a vine.
11. The grafted plant of embodiment 10, wherein the crop plant is selected from the group consisting of corn, soybean, cotton, canola, wheat, rice, tomato, tobacco, millet, potato, sugarbeet, cassava, alfalfa, barley, oats, sugarcane, sunflower, strawberry, and sorghum.
12. A progeny plant produced by a method comprising the steps of:
(a) obtaining a population of progeny plants from the grafted plant of embodiment 1, wherein the population of progeny plants is a first, second, or third generation of progeny plants obtained by selfing the grafted plant or by selfing the first or second generation progeny plants;
(b) screening the population of progeny plants for plants having improved yield or growth rate in comparison to control plants; and
(c) selecting a progeny plant from the population for an improvement in yield or growth rate in comparison to a control plant, wherein said progeny plant exhibits said improvement in yield or growth rate and exhibits a nuclear chromosomal DNA methylation pattern that is distinct from the control plant nuclear chromosomal DNA methylation pattern, and wherein the control plant is grown under the same environmental conditions as the selected progeny plants and comprises either: (i) progeny of a scion grafted to rootstock that had not been subjected to suppression of MSH1 and DRM2 gene expression; (ii) a whole plant that lacks any root graft and that had not been subjected to suppression of MSH1 gene expression; (iii) a wild-type plant; or (iv) progeny of a plant that is isogenic to the plant source of the scion of the grafted plant.
13. A selected population of progeny plants produced by a method comprising the steps of:
(a) obtaining a population of progeny plants from the grafted plant of embodiment 1, wherein the population of progeny plants is a first, second, or third generation of progeny plants obtained by selfing the grafted plant or by selfing the first or second generation progeny plants;
(b) screening the population of progeny plants for improved yield or growth rate in comparison to a control plant population; and
(c) selecting a population of progeny plants for an improvement in yield or growth rate in comparison to control plants, wherein said selected population of progeny plants exhibits said improvement in yield or growth rate and exhibits a nuclear chromosomal DNA methylation pattern that is distinct from a control plant nuclear chromosomal DNA methylation pattern, and wherein the control plant is grown under the same environmental conditions as the selected population of progeny plants and comprises either: (i) progeny of a scion grafted to rootstock that had not been subjected to suppression of MSH1 and DRM2 gene expression; (ii) a whole plant that lacks any root graft and that had not been subjected to suppression of MSH1 and DRM2 gene expression; (iii) a wild-type plant; or (iv) progeny of a plant that is isogenic to the plant source of the scion of the grafted plant.
14. The method of embodiment 13, wherein the MSH1 and DRM2 gene expression is suppressed in the rootstock of the grafted plant by a loss-of-function mutation in an endogenous MSH1 and DRM2 gene(s) of the rootstock, optionally wherein the grafted plant is a soybean, Brassica, tomato, sorghum, rice, or corn plant and the MSH1 and DRM2 gene(s) encode the cDNA(s) set forth in Table 1.
15. A method for producing a plant exhibiting a useful trait comprising the steps of:
(a) obtaining a population of progeny plants from a grafted plant comprising a scion to which a rootstock had been grafted, wherein the rootstock is obtained from a plant or a parent plant thereof had been subjected to suppression of MSH1 and DRM2 gene expression; and,
(b) selecting one or more progeny plants from the population, wherein the selected progeny plant exhibit an improvement in the useful trait in comparison to a control plant, thereby producing a plant that exhibits a useful trait.
16. The method of embodiment 15, wherein the population of progeny plants are obtained from seed of the grafted plant of step (a).
17. The method of embodiment 15, wherein the population of progeny plants are obtained from clonal propagates of the grafted plant of step (a).
18. The method of embodiment 15, 16, or 17, wherein plastid function has been recovered in the rootstock that is grafted to the scion in step (a).
19. The method of any one of embodiments 15 to 18, wherein the rootstock that is grafted to the scion in step (a) is obtained from a plant that was selected for the useful trait and that was derived from a parent plant that had been subjected to suppression of MSH1 and DRM2 gene expression.
20. The method of any of embodiments 15 to 19, wherein the useful trait is selected from the group consisting of improved yield, delayed flowering, non-flowering, increased biotic stress resistance, increased abiotic stress resistance, enhanced lodging resistance, enhanced growth rate, enhanced biomass, enhanced tillering, enhanced branching, delayed flowering time, delayed senescence, increased flower number, improved architecture for high density planting, improved photosynthesis, increased root mass, increased cell number, improved seedling vigor, improved seedling size, increased rate of cell division, improved metabolic efficiency, and increased meristem size in comparison to the control plant.
21. The method of any one of embodiments 15 to 20, wherein the scion contain(s) one or more epigenetic changes in one or more nuclear chromosomes, wherein the epigenetic changes are absent from nuclear chromosomes of the control plant or are absent from nuclear chromosomes of a plant from which the scion was obtained.
22. The method of embodiment 21, wherein the epigenetic change(s) are also present in the rootstock that had been subjected to perturbation of plastid function.
23. The method of embodiment 21 or 22, wherein the epigenetic changes are associated with the improvement in the useful trait.
24. The method of any one of embodiments 15 to 23, wherein the rootstock contain(s) one or more epigenetic changes in one or more nuclear chromosomes that are absent from nuclear chromosomes of rootstock obtained from a plant or are absent from nuclear chromosomes of a parent plant thereof had not been subjected to perturbation of plastid function.
25. The method of any one of embodiments 15 to 24, wherein said plant is selected from the group consisting of a crop plant, a tree, a bush, or a vine.
26. The method of embodiment 25, wherein the crop plant is selected from the group consisting of corn, Brassica, soybean, cotton, canola, wheat, rice, tomato, tobacco, millet, potato, sugarbeet, cassava, alfalfa, barley, oats, sugarcane, sunflower, strawberry, and sorghum.

Any order of steps described herein is merely illustrative and is not intended to limit the scope of the inventive concepts. For example, a step that is interpreted as being performed “first” may be performed as a first, second, third, or other step in a process. Similarly, any steps described as “following” other steps should not be interpreted to impart order, unless expressly indicated otherwise.

Although some example embodiments according to the inventive concepts may have been described with reference specific times, temperatures, materials, and/or other measurements, the inventive concepts should not be limited to these specific times, temperatures, materials and/or other measurements.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Use of Msh1 and Drm2 Loss-of-Function Mutations in Arabidopsis Rootstock and Effect on Graft Progeny

Plant materials. Experiments were conducted in Arabidopsis thaliana accession Col-0 with inclusion of the msh1 T-DNA mutant insertion line SAIL_877_F01 and drm2-2 mutant (CS16386; ABRC Stock Center). Clean seeds were sown on peat mix in square pots (3.5″×3.5″), with stratification at 4° C. for 2 days before transport to growth chambers (Percival AR-66L3; 22° C. day and 20° C. night, 12 hr DL, 120-150 μmol m−2 s−1 light).

Methods. Wedge-cleft grafting was performed using primary inflorescence meristems. Graft junctions were sealed with stretched parafilm to prevent desiccation and further secured using silicone tubing to help support contact between scion and rootstock. Grafted plants were kept under transparent plastic bags that acted as a mist chamber until scions started to grow, after which plants were slowly acclimatized to normal growth conditions. Additional floral shoots were removed to promote growth of the primary grafted floral stem. Each grafted scion was harvested separately, producing generation-one progeny.

Generation-one progenies were used to measure total leaf-area at different timepoints. For this, pictures were taken using Canon EOS Rebel XSi camera mounted on a copy stand (RS-CS920). Pictures were later analyzed using ImageJ software (version 1.52a; https://imagej.nih.gov/ij/index.html) to extract total leaf area. Mean leaf of each population was calculated and plotted using ggplot2 package (version 3.3.3) in RStudio (version 1.1.423).

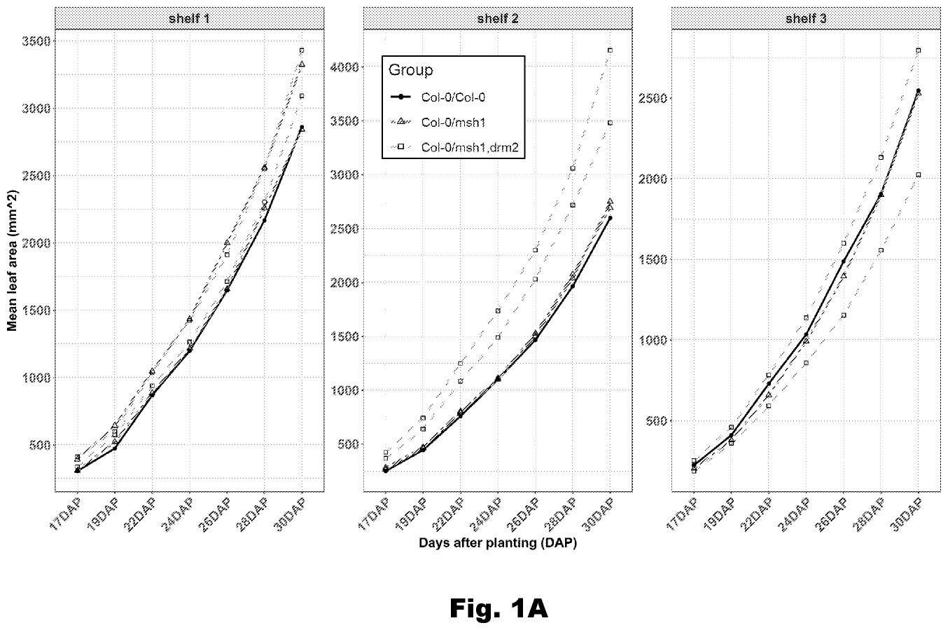

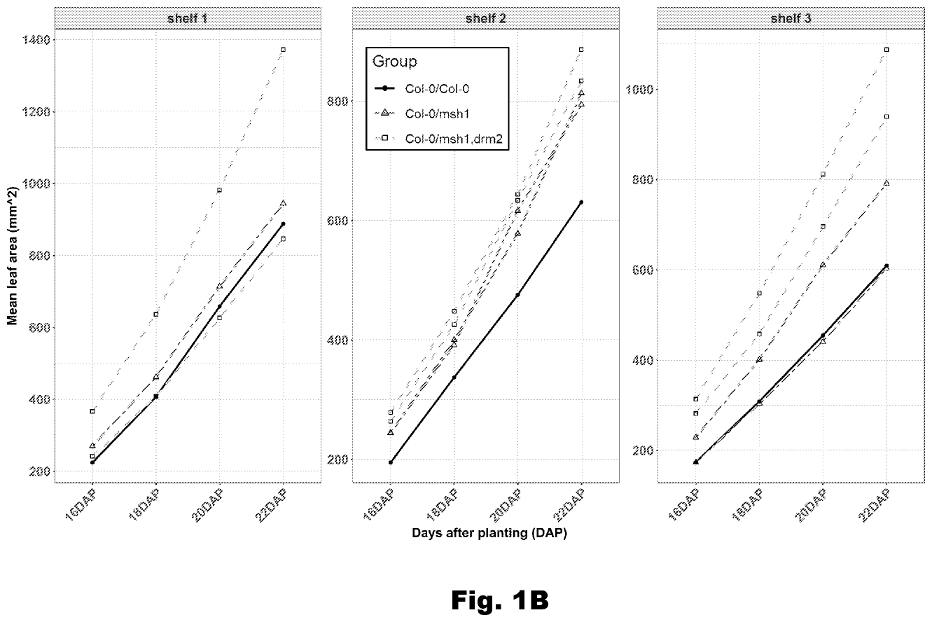

Grafting was conducted with inflorescence stems in Arabidopsis with Col-0 as scion and Col-0, msh1 (Col-0 background) or msh1,drm2 double mutant (Col-0 background) as rootstocks. Two experiments were conducted, with each including three Col-0, five msh1 and six msh1,drm2 rootstocks. Seed from each graft were collected and then planted in potting media at 18 seed/flat and grown under controlled growth conditions. Each experiment was conducted within a single growth chamber on three shelves, incorporating Col-0/Col-0 grafts on each shelf to control for chamber variation. Growth (rosette area) of the first-generation graft progeny were monitored and results are shown in FIG. 1.

In each experiment, five of the six progeny populations derived from Col-0/msh1,drm2 outperformed wild type (Col-0/Col-0) control grafts for plant growth rate. In contrast, Experiment 1 showed only one of five Col-0/msh1 graft progeny to outperform wild type, and Experiment 2 showed three of five Col-0/msh1 graft progeny to be enhanced in growth rate. The data suggest that incorporation of the drm2 mutation with msh1 to the rootstock improved frequency and magnitude of growth enhancement in graft progeny over the effects of msh1 alone.

We observed variation in growth rate on the different shelves of the chamber, suggesting that there was some variation in light conditions. The second experiment was terminated earlier than the first due to insect infestation that arose by Day 24. However, Experiment 1 showed that Day 22 data were strongly predictive of final outcomes, so we consider the variation observed in Experiment 2 data meaningful. We conducted the experiment with emphasis on plant growth rate rather than seed yield; we and others have not found Arabidopsis seed yield to be informative of yield outcomes in other crops (Van Daele et al. 2012).

From these data, we conclude that incorporation of the drm2 mutation with msh1 enhances rootstock performance to increase the frequency of growth enhancement in resulting progeny. Our results also suggest that the double mutant rootstock may result in increased magnitude of progeny growth enhancement.

### Example 2: Enhanced Growth Effect in Subsequent Generations

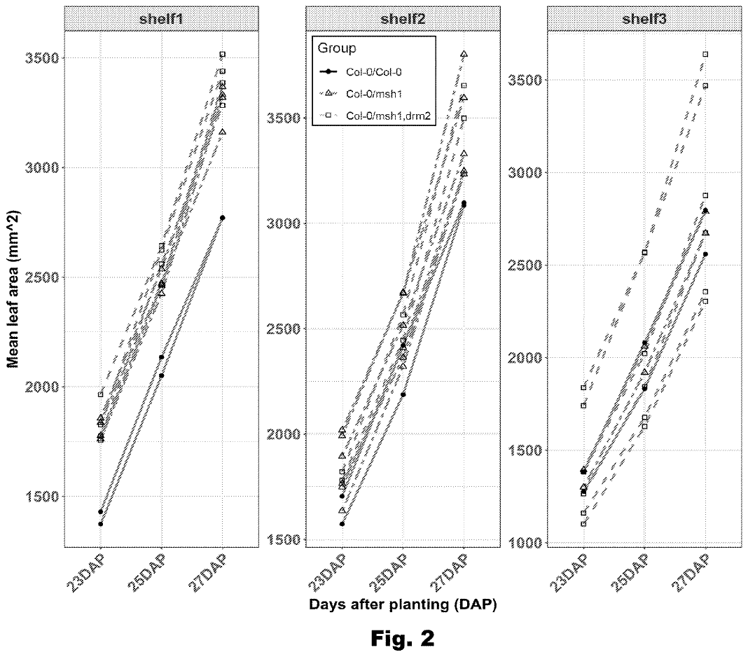

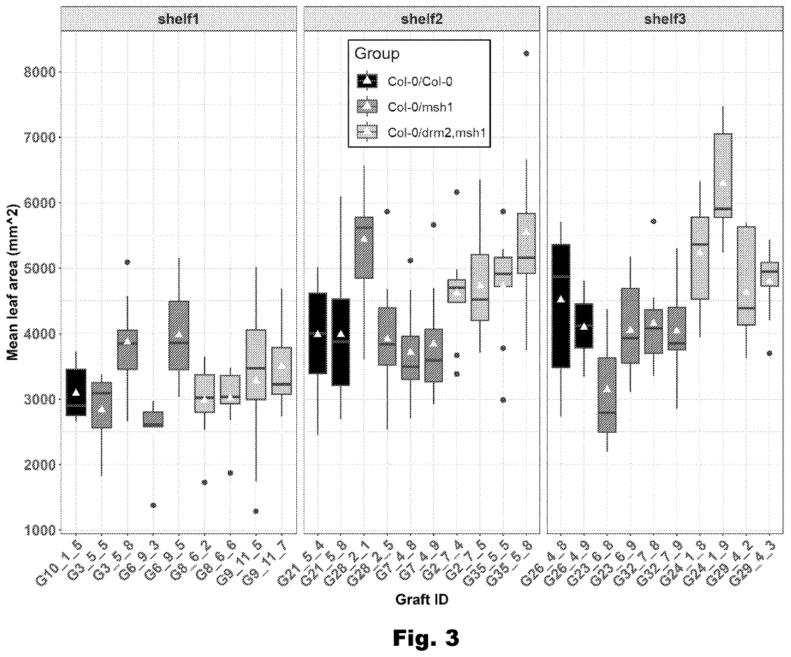

To test the longevity of enhanced growth, plants in first generation (FIG. 1) were self-pollinated to produce second generation plants, and subsequently second generation plants were self-pollinated to generate third generation plants. The total leaf area of second and third generation plants were analyzed similarly to the first generation as described in Example 1, and the results are shown in FIG. 2 and FIG. 3 respectively.

In the second generation, eight of the twelve progeny populations derived from Col-0/msh1,drm2 outperformed wild type (Col-0/Col-0) control grafts for plant growth rate. While ten of twelve Col-0/msh1 graft progeny populations outperformed wild type control grafts. In the third generation, seven of the twelve and three of the twelve progeny populations derived from Col-0/msh1,drm2 and Col-0/msh1, respectively, outperformed wild type control grafts.

From these data, we conclude that the enhanced growth displayed in generation one of Col-0/msh1,drm2 and Col-0/msh1 is heritable to at least generation three. These results also suggest that the double mutant rootstock may result in increased frequency of growth enhancement even in generation three.

### Example 3: Generation of B. napus Msh1,Drm2 Rootstock

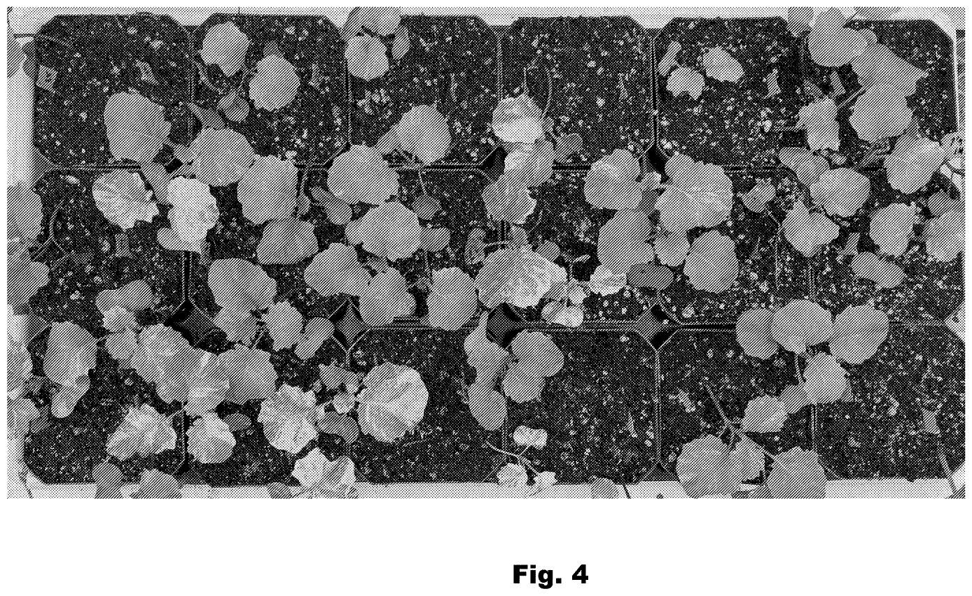

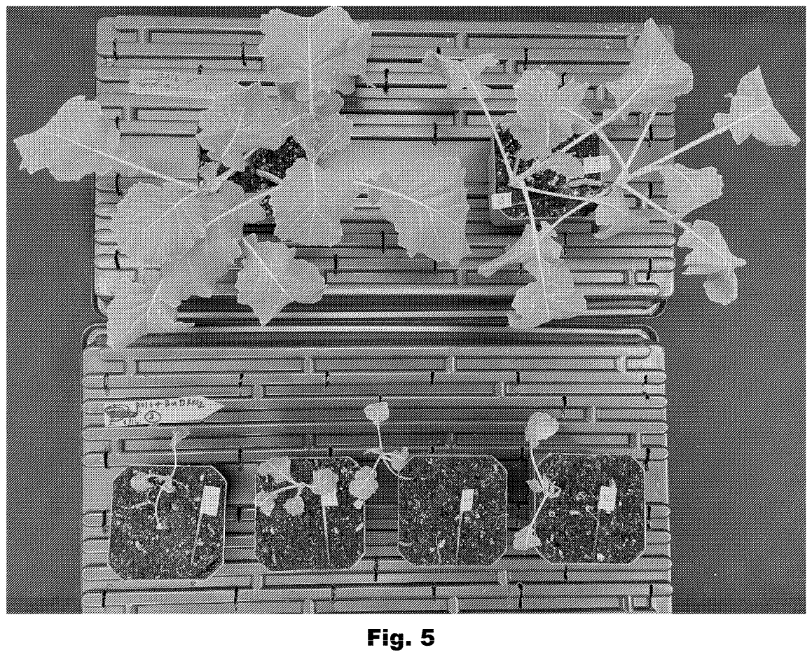

To incorporate msh1,drm2 rootstock grafting in crop, we used a CRISPR-cas9 technique to generate targeted mutations in MSH1 and DRM2 in Brassica napus (cv R016). To this end, we separately cloned one target (GCAGGCCACTGCACGTAGA, SEQ ID NO: 22) for MSH1 and two targets (target1: CGGTGGAACGTCGTACAAG, SEQ ID NO: 23; target2: ACAGAACGTTGATACCATA, SEQ ID NO: 24) for DRM2 into a pHSE401 vector (Xing et al 2014) containing gRNA and Cas9. Using the transformation protocol described by Cardoza et al 2006, CRISPR constructs were introduced into Brassica napus (cv R016). We successfully generated plants with a msh1 mutation displaying variegation and range in growth phenotype (FIG. 4), and drm2 mutant plants displaying a delayed growth phenotype (FIG. 5). To generate msh1, drm2 rootstock for grafting, we will be crossing msh1 and drm2 mutants plants and select for msh1,drm2 double mutants in F2 to generation. Msh1,drm2 rootstocks will then be used to graft WT scion as described in Example 1 and progenies will be field tested for enhanced yield.

### CITED REFERENCES

1. Bonasio, R., Tu, S. & Reinberg, D. (2010) Molecular signals of epigenetic states. Science 33:612-616
2. Mirouze, M. & Paszkowski, J. (2011) Epigenetic contribution to stress adaptation in plants. Curr Opin Plant Biol. 14:267-274
3. Dowen, R. H. et al. (2012) Widespread dynamic DNA methylation in response to biotic stress. Proc. Natl. Acad. Sci. USA 109: E2183-2191
4. Youngson, N. A. & Whitelaw, E. (2008) Transgenerational epigenetic effects. Annu. Rev. Genom. Human Genet 9:233-257
5. Paszkowski, J. & Grossniklaus, U. (2011) Selected aspects of transgenerational epigenetic inheritance and resetting in plants. Curr. Opin. Plant Biol. 14:195-203
6. Reinders, J. et al. (2009) Compromised stability of DNA methylation and transposon immobilization in mosaic Arabidopsis epigenomes. Genes Dev. 23:939-950
7. Johannes, F. et al. (2009) Assessing the impact of transgenerational epigenetic variation on complex traits. PLOS Genet. 5: e1000530
8. Roux, F. et al. (2011) Genome-wide epigenetic perturbation jump-starts patterns of heritable variation found in nature. Genetics 188:1015-1017.
9. Eichten, S. R. et al. (2011) Heritable epigenetic variation among maize inbreds. PLOS Genet. 7: e1002372.
10. Shen, H. et al. (2012) Genome-wide analysis of DNA methylation and gene expression changes in two Arabidopsis ecotypes and their reciprocal hybrids. Plant Cell 24:875-892
11. Becker, C. et al. (2011) Spontaneous epigenetic variation in the Arabidopsis thaliana methylome. Nature 480:245-249
12. Schmitz, R. J. et al. (2011) Transgenerational epigenetic instability is a source of novel methylation variants. Science 334:369-373
13. Abdelnoor, R. V. et al. (2003) Substoichiometric shifting in the plant mitochondrial genome is influenced by a gene homologous to MutS. Proc. Natl. Acad. Sci. USA 100:5968-5973
14. Xu, Y.-Z. et al. (2011) MutS HOMOLOG1 is a nucleoid protein that alters mitochondrial and plastid properties and plant response to high light. Plant Cell 23:3428-3441
15. Xu, Y.-Z. et al. (2012) The chloroplast triggers developmental reprogramming when MUTS HOMOLOG1 is suppressed in plants. Plant Physiol. 159:710-720
16. Lu, P. et al. (2012) Analysis of Arabidopsis genome-wide variations before and after meiosis and meiotic recombination by resequencing Landsberg erecta and all four products of a single meiosis. Genome Res. 22:508-518
17. Stokes, T. L., Kunkel, B. N. & Richards, E. J. (2002) Epigenetic variation in Arabidopsis disease resistance. Genes Dev 16:171-182
18. Lister, R. et al. (2008) Highly integrated single-base resolution maps of the epigenome in Arabidopsis. Cell 133:523-36
19. Hsieh, T.-F., et al. (2009) Genome-wide demethylation of Arabidopsis endosperm. Science 324:1451-1454
20. Gehring, M., Bubb, K. L. & Henikoff, S. (2009) Extensive demethylation of repetitive elements during seed development underlies gene imprinting. Science 324:1447-1451
21. Shedge, V., Arrieta-Montiel, M. P., Christensen, A. C. & Mackenzie, S. A. (2007) Plant mitochondrial recombination surveillance requires unusual RecA and MutS homologs. Plant Cell 19:1251-1264
22. Shedge, V., Davila, J., Arrieta-Montiel, M. P., Mohammed, S. & Mackenzie S. A. (2010) Extensive rearrangement of the Arabidopsis mitochondrial genome elicits cellular conditions for thermotolerance. Plant Physiol. 152:1960-1970
23. Kalisz, S. & Kramer, E. M. (2008) Variation and constraint in plant evolution and development. Hered. 100:171-177
24. Greaves, I., Groszmann, M., Dennis, E. S. & Peacock, W. J. (2012) Trans-chromosomal methylation. Epigenetics 7:800-805
25. Shivaprasad, P. V., Dunn, R. M., Santos, B. A., Bassett, A. & Baulcombe, D. C. (2012) Extraordinary transgressive phenotypes of hybrid tomato are influenced by epigenetics and small silencing RNAs. EMBO J 31:257-266
26. McMullen M. D., et al. (2009) Genetic properties of the maize nexted association mapping population. Science 7:737-740
27. Notredame, C., Higgins, D. G. & Heringa, J. (2000) T-Coffee: A novel method for fast and accurate multiple sequence alignment. J Mol. Biol. 302:205-217
28. Krueger, F. & Andrews, S. R. (2011) Bismark: a flexible aligner and methylation caller for Bisulfite-Seq applications. Bioinformatics 27:1571-1572
29. Storey, J. D. & Tibshirani, R. (2003) Statistical significance for genome-wide studies. Proc. Natl. Acad. Sci. USA 100:9440-9445
30. Bolstad, B., Irizarry, R. A., Astrand, M. & Speed T. (2003) A comparison of normalization methods for high density oligonucleotide array data based on bias and variance. Bioinformatics 19:195-193
31. Smyth, G. K. (2004) Linear models and empirical Bayes methods for assessing differential expression in microarray experiments. Stat. Appl. Genet. Mol. Biol. 3: Article 3
32. Huang, D. W., Sherman, B. T. & Lempicki, R. A. (2009) Systematic and integrative analysis of large gene lists using DAVID Bioinformatics Resources. Nat. Protoc. 4:44-57
33. Martin M. (2011) Cutadapt removes adapter sequences from high-throughput sequencing reads. EMBnet Journal, Vol 17, No 1.
34. Langmead, B. & Salzberg, S. NGS QC Toolkit: A toolkit for quality control of next generation sequencing data. PLOS ONE 7 (2): e30619
35. Hannon Lab. FASTX-Toolkit. On the internet at “hannonlab.cshl.edu/fastx toolkit/”
36. Zerbino D R, McEwen G K, Margulies E H, Birney E. (2009) Pebble and Rock Band: Heuristic Resolution of Repeats and Scaffolding in the Velvet Short-Read de Novo Assembler. PLOS ONE 4 (12): e8407
37. Camacho, C. et al. (2012) BLAST+: architecture and applications. BMC Bioinformatics 10, 421 (2009). Fast gapped-read alignment with Bowtie 2. Nat. Methods 9:357-359
38. Li, H. et al. (2009) The Sequence alignment/map (SAM) format and SAMtools. Bioinformatics 25:2078-2079.
39. Kechanmane Raju, S. K., Shao M.-R., Sanchez, R, Xu Y.-Z., Sandhu A, Graef G, Mackenzie, S. 2018. An epigenetic breeding system in soybean for increased yield and stability. Plant Biotech J 16 (11): 1836-1847
40. Kundariya, H, Sanchez, R, Yang, X, Hafner, A, Mackenzie, S A. 2022. Methylome decoding of RdDM-mediated reprogramming effects in the Arabidopsis MSH1 system as model. Genome Biology, revised pending acceptance.
41. Kundariya H, Yang Y, Morton K, Sanchez R, Axtell M J, Hutton S F, Fromm M, Mackenzie S A. 2020. MSH1-induced heritable enhanced growth vigor through grafting is associated with the RdDM pathway in plants. Nature Communications 11:5345
42. Shao M.-R., Kumar, S. K. R., Laurie, J. D., Sanchez, R., Mackenzie, S A. 2017. Stress-responsive pathways and small RNA changes distinguish variable developmental phenotypes caused by MSH1 loss. BMC Plant Biology 17:47.
43. Van Daele I, Gonzalez N, Vercauteren I, de Smet L, Inzé D, Roldán-Ruiz I, Vuylsteke M. A comparative study of seed yield parameters in Arabidopsis thaliana mutants and transgenics. Plant Biotechnol J. 2012 May; 10 (4): 488-500.
44. Virdi K S, Laurie J D, Xu Y-Z, Wang D, Yu J, Shao M-R, Sanchez R, Feng S, Kundariya H, Wamboldt Y, Chen M, Riethoven J-J M, Arrieta-Montiel M P, Mackenzie S A. 2015. Arabidopsis MSH1 mutation alters the epigenome to produce heritable changes in plant growth. Nature Communications 6:6386.
45. Yang X, Kundariya H, Xu Y Z, Sandhu A, Hutton S F, Zhang M, Mackenzie S A. 2015. MSH1-derived epigenetic breeding potential in tomato. Plant Physiology 168:222-32.
46. Yang, X, Sanchez R, Kundariya H, Maher T, Dopp I, Schwegel R, Virdi K, Axtell M J, Mackenzie S A. 2020. Segregation of an MSH1 RNAi transgene produces heritable non-genetic memory in association with methylome reprogramming. Nat Commun. 11 (1): 2214
47. Xing, H L., Dong, L., Wang, Z P. et al. A CRISPR/Cas9 toolkit for multiplex genome editing in plants. BMC Plant Biol 14, 327 (2014). doi.org/10.1186/s12870-014-0327-y
48. Cardoza, V., Neal Stewart, C. (2006). Canola (Brassica napus L.). In: Wang, K. (eds) Agrobacterium Protocols. Methods in Molecular Biology, vol 343. Humana Press. https://doi.org/10.1385/1-59745-130-4:257

What is claimed is:

1. A grafted plant comprising a scion to which a rootstock had been grafted, wherein:
(i) the scion is from a wild type plant;
(ii) MSH1 and DRM2 gene expression is suppressed in the rootstock;
(iii) the rootstock confers an improvement in yield or growth rate in progeny of the grafted plant in comparison to a control plant, wherein the control plant comprises either: (a) progeny of a scion grafted to rootstock that had not been subjected to suppression of MSH1 and DRM2 gene expression; (b) a whole plant that lacks any root graft and that had not been subjected to suppression of MSH1 and DRM2 gene expression; (c) a wild-type plant; or (d) progeny of a plant that is isogenic to the plant source of the scion of the grafted plant.

2. The grafted plant of claim 1, wherein the rootstock confers to the grafted plant an improvement in yield or growth rate in comparison to a control plant.

3. The grafted plant of claim 1, wherein the scion contains one or more epigenetic changes in one or more nuclear chromosomes, wherein the epigenetic changes are absent from the nuclear chromosomes of a control plant or are absent from nuclear chromosomes of a plant from which the scion was obtained.

4. The grafted plant of claim 3, wherein the epigenetic change(s) are also present in the rootstock.

5. The grafted plant of claim 3, wherein the epigenetic changes are associated with the improvement in the useful trait.

6. The grafted plant of claim 3, wherein the rootstock contain(s) one or more epigenetic changes in one or more nuclear chromosomes that are absent from nuclear chromosomes of rootstock obtained from a plant or nuclear chromosomes of a parent plant thereof that had not been subjected to suppression of MSH1 and DRM2 gene expression.

7. The grafted plant of claim 1, wherein the MSH1 and DRM2 gene expression is suppressed in the rootstock by a loss-of-function mutation in an endogenous MSH1 and/or DRM2 gene of the rootstock and/or by a small inhibitory RNA (siRNA), a microRNA (miRNA), a co-suppressing sense RNA, and/or an anti-sense RNA having complementarity to the endogenous MSH1 and/or DRM2 gene promoter, 5′ or 3′ untranslated region, intron, coding region, and/or any combination thereof, optionally wherein the grafted plant is a soybean, Brassica, tomato, sorghum, rice, or corn plant and the endogenous MSH1 and DRM2 gene(s) encode the cDNA(s) set forth in Table 1.

8. The grafted plant of claim 7, wherein the MSH1 and DRM2 gene expression is suppressed in the rootstock by a loss-of-function mutation in an endogenous MSH1 and DRM2 gene(s), optionally wherein the grafted plant is a soybean, Brassica, tomato, sorghum, rice, or corn plant and the MSH1 and DRM2 gene(s) encode the cDNA(s) set forth in Table 1.

9. The grafted plant of claim 7, wherein the MSH1 and/or DRM2 gene expression is suppressed in the rootstock by a small inhibitory RNA (siRNA), a microRNA (miRNA), a co-suppressing sense RNA, and/or an anti-sense RNA having complementarity to the endogenous MSH1 and/or DRM2 gene promoter, 5′ or 3′ untranslated region, intron, coding region, and/or any combination thereof, optionally wherein the grafted plant is a soybean, Brassica, tomato, sorghum, rice, or corn plant and the MSH1 and DRM2 gene(s) encode the cDNA(s) set forth in Table 1.

10. The grafted plant of claim 1, where said plant is selected from the group consisting of a crop plant, a tree, a bush, turf grass, pasture grass, and a vine.

11. The grafted plant of claim 10, wherein the crop plant is selected from the group consisting of corn, soybean, cotton, canola, wheat, rice, tomato, tobacco, millet, potato, sugarbeet, cassava, alfalfa, barley, oats, sugarcane, sunflower, strawberry, and sorghum.

12. A progeny plant produced by a method comprising the steps of:
(a) obtaining a population of progeny plants from the grafted plant of claim 1, wherein the population of progeny plants is a first, second, or third generation of progeny plants obtained by selfing the grafted plant or by selfing the first or second generation progeny plants;
(b) screening the population of progeny plants for plants having improved yield or growth rate in comparison to control plants; and
(c) selecting a progeny plant from the population for an improvement in yield or growth rate in comparison to a control plant, wherein said progeny plant exhibits said improvement in yield or growth rate and exhibits a nuclear chromosomal DNA methylation pattern that is distinct from the control plant nuclear chromosomal DNA methylation pattern, and wherein the control plant is grown under the same environmental conditions as the selected progeny plants and comprises either: (i) progeny of a scion grafted to rootstock that had not been subjected to suppression of MSH1 and DRM2 gene expression; (ii) a whole plant that lacks any root graft and that had not been subjected to suppression of MSH1 gene expression; (iii) a wild-type plant; or (iv) progeny of a plant that is isogenic to the plant source of the scion of the grafted plant.

13. A selected population of progeny plants produced by a method comprising the steps of:
(a) obtaining a population of progeny plants from the grafted plant of claim 1, wherein the population of progeny plants is a first, second, or third generation of progeny plants obtained by selfing the grafted plant or by selfing the first or second generation progeny plants;
(b) screening the population of progeny plants for improved yield or growth rate in comparison to a control plant population; and
(c) selecting a population of progeny plants for an improvement in yield or growth rate in comparison to control plants, wherein said selected population of progeny plants exhibits said improvement in yield or growth rate and exhibits a nuclear chromosomal DNA methylation pattern that is distinct from a control plant nuclear chromosomal DNA methylation pattern, and wherein the control plant is grown under the same environmental conditions as the selected population of progeny plants and comprises either: (i) progeny of a scion grafted to rootstock that had not been subjected to suppression of MSH1 and DRM2 gene expression; (ii) a whole plant that lacks any root graft and that had not been subjected to suppression of MSH1 and DRM2 gene expression; (iii) a wild-type plant; or (iv) progeny of a plant that is isogenic to the plant source of the scion of the grafted plant.

14. The method of claim 13, wherein the MSH1 and DRM2 gene expression is suppressed in the rootstock of the grafted plant by a loss-of-function mutation in an endogenous MSH1 and DRM2 gene(s) of the rootstock, optionally wherein the grafted plant is a soybean, Brassica, tomato, sorghum, rice, or corn plant and the MSH1 and DRM2 gene(s) encode the cDNA(s) set forth in Table 1.

15. A method for producing a plant exhibiting a useful trait comprising the steps of:
(a) obtaining a population of progeny plants from a grafted plant comprising a scion to which a rootstock had been grafted, wherein the rootstock is obtained from a plant or a parent plant thereof had been subjected to suppression of MSH1 and DRM2 gene expression, and,
(b) selecting one or more progeny plants from the population, wherein the selected progeny plant exhibit an improvement in the useful trait in comparison to a control plant, thereby producing a plant that exhibits a useful trait.

16. The method of claim 15, wherein the population of progeny plants are obtained from seed of the grafted plant of step (a).

17. The method of claim 15, wherein the population of progeny plants are obtained from clonal propagates of the grafted plant of step (a).

18. The method of claim 15, wherein plastid function has been recovered in the rootstock that is grafted to the scion in step (a).

19. The method of claim 15, wherein the rootstock that is grafted to the scion in step (a) is obtained from a plant that was selected for the useful trait and that was derived from a parent plant that had been subjected to suppression of MSH1 and DRM2 gene expression.

20. The method of claim 15, wherein the useful trait is selected from the group consisting of improved yield, delayed flowering, non-flowering, increased biotic stress resistance, increased abiotic stress resistance, enhanced lodging resistance, enhanced growth rate, enhanced biomass, enhanced tillering, enhanced branching, delayed flowering time, delayed senescence, increased flower number, improved architecture for high density planting, improved photosynthesis, increased root mass, increased cell number, improved seedling vigor, improved seedling size, increased rate of cell division, improved metabolic efficiency, and increased meristem size in comparison to the control plant.

21. The method of claim 20, wherein the scion contain(s) one or more epigenetic changes in one or more nuclear chromosomes, wherein the epigenetic changes are absent from nuclear chromosomes of the control plant or are absent from nuclear chromosomes of a plant from which the scion was obtained.

22. The method of claim 21, wherein the epigenetic change(s) are also present in the rootstock that had been subjected to perturbation of plastid function.

23. The method of claim 21, wherein the epigenetic changes are associated with the improvement in the useful trait.

24. The method of claim 21, wherein the rootstock contain(s) one or more epigenetic changes in one or more nuclear chromosomes that are absent from nuclear chromosomes of rootstock obtained from a plant or are absent from nuclear chromosomes of a parent plant thereof had not been subjected to perturbation of plastid function.

25. The method of claim 15, wherein said plant is selected from the group consisting of a crop plant, a tree, a bush, or a vine.

26. The method of claim 25, wherein the crop plant is selected from the group consisting of corn, Brassica, soybean, cotton, canola, wheat, rice, tomato, tobacco, millet, potato, sugarbeet, cassava, alfalfa, barley, oats, sugarcane, sunflower, strawberry, and sorghum.
